(19) United States
(12) Patent Application Publication
(10) Pub. No.: US 2003/0113897 A1
(43) Pub. Date: Jun. 19, 2003

(54) Mutant p21Cip1/WAF1 and cell growth control and cell growth control

(76) Inventors: Mien-Chie Hung, Houston, TX (US); Binhua P. Zhou, Houston, TX (US)

Correspondence Address: FULBRIGHT & JAWORSKI, LLP, 1301 MCKINNEY, SUITE 5100, HOUSTON, TX, 77010-3095, US

(21) Appl. No.: 10/142,174

(22) Filed: May 9, 2002

Related U.S. Application Data
(60) Provisional application No. 60/289,651, filed on May 9, 2001.

(51) Int. Cl. C12N 9/64; C07H 21/04; C12P 21/02; C12N 5/06

(52) U.S. Cl. 435/226; 435/69.1; 435/320.1; 435/325; 536/23.2

(57) ABSTRACT

Disclosed are methods and compositions regarding separate mutant forms of p21^{Cip1/WAF1} that are associated with control of cell growth. Substitution of Thr¹⁴⁵ with another amino acid, such as Ala, results in failure to be phosphorylated at that site and leads to retention of the polypeptide in the nucleus, resulting in preferentially suppressing growth of transformed cells. Alternatively, substitution of Thr¹⁴⁵ with another amino acid, such as Asp, results in cytoplasmic translocation of the polypeptide and results in enhancing cellular survival.

This application claims priority to U.S. Provisional Patent Application Serial No. 60/289,651, filed May 9, 2001, incorporated by reference herein in its entirety.

### FIELD OF THE INVENTION

The present invention is directed to methods and compositions regarding cell and molecular biology. More specifically, the present invention is directed to proliferation diseases, such as cancer, and control of cell growth. Most specifically, the present invention is directed to mutant forms of p21^{Cip1/WAF1} in either growth inhibition or promotion of cellular survival.

### BACKGROUND OF THE INVENTION

The cell cycle and the genetic alterations that drive tumorigenesis are inextricably linked. Examples include the amplification of cyclin and cyclin dependent kinase (CDK) genes, the phosphorylation of Rb by CDKs, the control of the CDK inhibitor p2^{WAF1} by p53, and the tumor suppressor activity of the CDK inhibitor p16 (reviewed by Sherr, 1994, Hunter and Pines, 1994).

A turning point in cell cycle research was realized with the discovery that cyclin complexes had different constitutions in transformed and nontransformed cells (Xiong et al., 1992, Xiong et al., 1993). In particular, the complexes in the non-transformed cells were associated with small proteins different from those found in transformed derivatives. A variety of approaches subsequently determined that these small proteins not only bound to the cyclin complexes, but were potent inhibitors of the associated CDKs (p21: Harper et al., 1993, Xiong et al., 1993b, Gu et al., 1993, p16: Serrano et al., 1993, p15: Hannon and Beach, 1994, p18: Guan et al., 1994, p27: Polyak et al, 1994, Toyoshima and Hunter, 1994).

Five CDK inhibitors have thus far been identified. The first to be cloned, p21^{WAF1} (also known as CIP1, SDI1, MDA6, CAP20 and hereafter referred to as p21^{Cip1/WAF1}), is encoded by a gene on chromosome 6p and can be directly regulated by p53 (El-Deiry et al., 1993). It inhibits a broad range of cyclin-CDK complexes, and may be involved in cellular senescence as well as in neoplasia (Noda et al., 1994). In vitro, p21^{Cip1/WAF1} also complexes with the proliferating cell nuclear antigen (PCNA), resulting in an inhibition of DNA replication (Li et al., 1994, Flores-Rozas et al., 1994). A related gene, p27^{KIP1}, is located on chromosome 12p and may play a role in a subset of leukemias (Bullrich et al., 1995, Pietenpol et al., 1995, Ponce-Castaneda et al., 1995). The p16, p15 and p18 genes are unrelated to the p21/p27 family, and have a more selective inhibitory activity, affecting only CDK4 and 6. The p16 and p15 genes are homozygously deleted in many human cancers, and p16 mutations result in familial melanoma (Kamb et al., 1994 a,b, Nobori et al., 1994, Hussussian et al., 1994, Jen et al., 1994).

The p21^{Cip1/WAF1} gene product has recently been associated with the formation of cyclin D-Cdk4 complexes and their nuclear translocation. The dual behavior of p21^{Cip1/WAF1}, both as an inhibitor of cyclin-dependent kinases and in association with cyclin D-Cdk4 complexes is likely the result of association with different proteins. As described by Taules et al. (1999), p21^{Cip1/WAF1}, and particularly its carboxy-terminal region, associates with calmodulin. The vast majority of studies involving p21^{Cip1/WAF1} have focused on its transcriptional regulation by p53-dependent and -independent mechanisms (Gartel and Tyner, 1999). Recent evidence indicates that p21^{Cip1/WAF1} can also be regulated post-translationally. The best characterized of these post-translational mechanisms is C-terminal cleavage of p21^{Cip1/WAF1} by a protease from the caspase family (Park et al., 1998, Gervais et al., 1998; Levkau et al., 1998; Zhang et al., 1999). The cleavage is an early event during DNA damage-induced apoptosis that affects both cellular localization (Levkau et al., 1998) and PCNA binding (Gervais et al., 1998; Zhang et al., 1999) of p21^{Cip1/WAF1}. Also, recent evidence suggests reversible phosphorylation is a mechanism for regulating p21^{Cip1/WAF1}. The most compelling data addresses p21^{Cip1/WAF1} during cellular differentiation (Erhardt and Pittman, 1998) Stimulation of PC12 cells to differentiate with nerve growth factor led to loss of epitope binding by a monoclonal antibody for C terminus-specific anti-p21^{Cip1/WAF1}, although no overall change in p21^{Cip1/WAF1} protein levels was apparent. However, phosphatase treatment of the lysates resulted in recovery of the epitope, indicating that reversible phosphorylation within the C-terminal region of p21^{Cip1/WAF1} occurs in response to nerve growth factor-stimulated signaling pathways. However, potential phosphorylation sites on p21^{Cip1/WAF1} were not identified by Erhardt and Pittman (1998), nor were potential biochemical effects of this phosphorylation on p21^{Cip1/WAF1} activity identified. The relatively low level expression of p21^{Cip1/WAF1} protein in cultured cells has made it difficult to map such regulatory phosphorylation sites on p21^{Cip1/WAF1} protein, and this precludes elucidation of post-translational regulatory mechanisms.

U.S. Pat. Nos. 5,807,692 and 5,871,968 are directed to utilizing p21^{Cip1/WAF1} expression, which is aberrant in neoplastic tissues, as a tool to assess neoplasia and to discover new drugs. Specifically, U.S. Pat. No. 5,871,968 relates to an in vitro method for expressing a gene in a cell which expresses p21^{Cip1/WAF1} by administering a nucleic acid construct having the p21^{Cip1/WAF1} promoter region linked in cis to the gene. However, this patent does not teach modifications to p21^{Cip1/WAF1} polypeptides for treatments of disease. U.S. Pat. No. 5,807,692 regards truncated p21^{Cip1/WAF1} proteins, particularly having the N-terminal 78 amino acids, which are useful in inhibiting tumor cell growth, although this patent does not teach substitution of particular amino acids in the polypeptide for tumor cell growth inhibition and enhancement of cell survival.

U.S. Pat. Nos. 5,863,904 and 6,057,300 are directed to inhibition of growth of malignant cells and the treatment of restenosis in vivo by expression of p21^{Cip1/WAF1}. Specifically, U.S. Pat. No. 5,863,904 addresses a method of treating restenosis by introducing a p21^{Cip1/WAF1} gene by catheter or direct injection into a blood vessel at the site of restenosis. U.S. Pat. No. 6,057,300 regards nucleic acid sequences encoding truncated p21^{Cip1/WAF1} proteins methods using the compositions to pre-screen for therapeutic agents for use in regulating cell growth by regulating p21^{Cip1/WAF1} expression. Neither of these patents are directed to compositions and methods for both promoting nuclear retention associated with inhibition of tumor cell proliferation and promotion of cytoplasmic translocation for cell survival enhancement by modifications to the p21^{Cip1/WAF1} polypeptide.

Scott et al. (2000) identify phosphorylation sites at Thr¹⁴⁵ and Ser 146 within a C-terminal regulatory domain, and modification in vivo modulates p21-PCNA associations. Scott et al. also refers to Rousseau et al. (1999), which shows that an intact C-terminus is required for nuclear localization of p21^{Cip1/WAF1}. However, Scott et al. does not provide any guidance with respect to how to modify the phosphorylation site to achieve either a p21^{Cip1/WAF1} polypeptide which is retained in the nucleus, and/or a p21^{Cip1/WAF1} polypeptide which cytoplasmically translocalizes. Furthermore, Scott et al. fails to address how different modifications of p21^{Cip1/WAF1} polypeptide result in alternative effects on cell growth.

### SUMMARY OF THE INVENTION

The present invention is directed to methods and compositions regarding discrete mutant forms of p21^{Cip1/WAF1} which are associated with control of cell growth, survival or proliferation. In specific embodiments, the control of cell growth is useful in the treatment of cancer or restenosis or is useful to block angiogenesis. A skilled artisan recognizes based on the teachings provided herein that the compositions and methods are useful to treat vascular cell/blood vessel proliferation, such as in angiogenesis. In specific embodiments, the angiogenesis is associated with diseases such as cancer, arthritis, atherosclerosis, blindness due to diabetes, and so forth. Specifically, the present invention teaches a skilled artisan that particular mutant p21^{Cip1/WAF1} polypeptides that have an amino acid substitution result in the polypeptide either not being phosphorylated and retained in the nucleus or p21^{Cip1/WAF1} polypeptide having substitutions which result in cytoplasmic translocation and enhancement of cell survival. More specifically, substitution of Thr¹⁴⁵ with another amino acid, such as Ala, results in failure of the polypeptide to be phosphorylated at that site and leads to retention of the polypeptide in the nucleus, resulting in preferentially suppressing growth of transformed cells. Alternatively, substitution of Thr¹⁴⁵ with another amino acid, such as Asp, results in cytoplasmic translocation of the polypeptide and subsequent enhancement of cellular survival.

Thus, the present invention provides guidance regarding different mutations in p21^{Cip1/WAF1} having different effects, and therefore the present invention is directed to a novel improvement to the overall arts of cell growth control, including inhibition of cell proliferation and, alternatively, enhancement of cell survival.

Therefore, an object of the present invention is directed to at least one modification in p21^{Cip1/WAF1} polypeptide which results in failure of the polypeptide to be phosphorylated and is subsequently retained in the nucleus. In a preferred embodiment, this nuclear retention occurs despite action of a kinase in the cell which would otherwise phosphorylate an unmodified p21^{Cip1/WAF1} polypeptide. In a more preferred embodiment, this nuclear retention occurs following activation of Akt. In a most preferred embodiment, this nuclear retention occurs following HER-2/neu mediated activation of Akt.

Another object of the present invention regards mutations in p21^{Cip1/WAF1} that result in cytoplasmic translocation of the polypeptide, such as modifications to at least one amino acid in a p21^{Cip1/WAF1} polypeptide in which the cytoplasmic translocation is associated with enhancement of survival of the cell.

A skilled artisan recognizes that any site in the p21^{Cip1/WAF1} polypeptide may be modified to generate such compositions as described, and furthermore that multiple sites may be modified. A skilled artisan is cognizant that a limited number of sites for modification exist in the approximately 160 amino acid p21^{Cip1/WAF1} polypeptide (depending on the organism). In addition, a skilled artisan recognizes that there are only twenty standard amino acids from which to modify to, and guidance is provided herein directed to methods to generate those modifications. Furthermore, a skilled artisan in the teachings of the present invention knows how to test for either nuclear retention or cytoplasmic translocation of a modified p21^{Cip1/WAF1} polypeptide, and therefore assaying a particular modification would not subject one skilled in the art to undue experimentation.

Based on the teachings of the present invention, a skilled artisan is cognizant of at least three outcomes following modification of at least one amino acid of the p21^{Cip1/WAF1} polypeptide: 1) the polypeptide is not phosphorylated in at least one site which is phosphorylated in the unmodified polypeptide, resulting in retention in the nucleus, and in a specific embodiment, inhibits cell proliferation; 2) the polypeptide is phosphorylated and/or mimics phosphorylation, resulting in cytoplasmic translocalization, and, in a specific embodiment, enhances cell survival; and 3) no effect is seen on the polypeptide.

Thus, based on the guidance provided herein, the present invention is directed to polypeptides of p21^{Cip1/WAF1} which are modified and result in inhibition of ,proliferation of a cell or enhancement of cell survival. In two specific embodiments, the present invention is directed to specific mutants including, for example, p21^{Cip1/WAF1} T145D and p21^{Cip1/WAF1} T145A.

Thus, in accordance with the objects of the present invention, there is as a composition of matter a p21^{Cip1/WAF1} polypeptide comprising an amino acid substitution at Thr¹⁴⁵. In a specific embodiment, the substitution prevents phosphorylation of the p21^{Cip1/WAF1} polypeptide under conditions that would result in phosphorylation of an unsubstituted p21^{Cip1/WAF1} polypeptide. In other specific embodiments, the substitution is a Thr¹⁴⁵ to Ala¹⁴⁵ substitution or a Thr¹⁴⁵ to Asp¹⁴⁵ substitution. In a specific embodiment, the substitution results in nuclear accumulation of the p21^{Cip1/WAF1} polypeptide following activation of Akt under conditions in which an unsubstituted p21^{Cip1/WAF1} polypeptide would translocate from the nucleus to the cytoplasm of a cell. In other specific embodiments, the compositions are further defined as compositions in a pharmacologically acceptable excipient in which the p21^{Cip1/WAF1} polypeptide is dispersed. In additional specific embodiments, the compositions are confined in a suitable container in a kit.

In another object of the present invention there is as a composition of matter a p21^{Cip1/WAF1} polypeptide comprising at least one modification which prohibits phosphorylation of the polypeptide under conditions that would result in phosphorylation of an unsubstituted p21^{Cip1/WAF1} polypeptide, wherein the modification results in accumulation of the polypeptide in a nucleus of a cell under conditions in which the unsubstituted p21^{Cip1/WAF1} polypeptide would translocate from the nucleus to the cytoplasm of the cell. In a specific embodiment, the modification is an amino acid substitution at Thr¹⁴⁵.

In another object of the present invention there is a method of preventing cytoplasmic translocation of p21^{Cip1/WAF1} polypeptide from a nucleus of a cell following activation of Akt, comprising the step of administering to said cell a p21^{Cip1/WAF1} polypeptide having an amino acid substitution at Thr¹⁴⁵. In a specific embodiment, substitution is a Thr¹⁴⁵ to Ala¹⁴⁵ substitution.

In an additional object of the present invention, there is a method of preventing growth of a cell in an individual comprising the step of administering to said individual a p21^{Cip1/WAF1} polypeptide having an amino acid substitution at Thr¹⁴⁵. In a specific embodiment, the substitution is a Thr¹⁴⁵ to Ala¹⁴⁵ substitution. In another specific embodiment, the administration of said polypeptide is by a liposome. In an additional specific embodiment, the polypeptide further comprises a protein transduction domain.

In another object of the present invention there is a method of preventing growth of a cell in an individual comprising the step of administering to said individual a nucleic acid encoding a p21^{Cip1/WAF1} polypeptide having an amino acid substitution at Thr¹⁴⁵. In a further specific embodiment, the substitution is a Thr¹⁴⁵ to Ala¹⁴⁵ substitution. In an additional specific embodiment, the Thr¹⁴⁵ is substituted with an amino acid which is not aspartic acid, glutamic acid, or serine. In another specific embodiment, the administration of said nucleic acid is by a vector selected from the group consisting of a plasmid, a retroviral vector, an adenoviral vector, an adeno-associated viral vector, a liposome, and a combination thereof.

In an additional object of the present invention, there is a method of using a p21^{Cip1/WAF1} polypeptide composition in which the polypeptide comprises an amino acid substitution at Thr¹⁴⁵, wherein the p21^{Cip1/WAF1} polypeptide composition is dispersed in a pharmacologically acceptable excipient, and wherein the composition is administered to an animal having a proliferative cell disorder.

In another object of the present invention, there is a method of treating a proliferative cell disorder in an individual comprising the step of administering to said individual a p21^{Cip1/WAF1} polypeptide having an amino acid substitution at Thr¹⁴⁵. In a specific embodiment, the substitution is a Thr¹⁴⁵ to Ala¹⁴⁵ substitution. In another specific embodiment, the proliferative cell disorder is cancer. In a further specific embodiment, the proliferative cell disorder is restenosis. In an additional specific embodiment, the cancer is associated with HER-2/neu-mediated cell proliferation. In a further specific embodiment, the cancer is breast cancer.

In an additional object of the preent invention, there is a method of increasing cell survival in an individual, comprising the step of administering to said individual a p21^{Cip1/WAF1} polypeptide having a Thr¹⁴⁵ to Asp¹⁴⁵ substitution or a Thr¹⁴⁵ to Glu¹⁴⁵ substitution.

In another object of the present invention there is a method of treating an individual for a degenerative disease, comprising the step of administering to said individual a p21^{Cip1/WAF1} polypeptide having a Thr¹⁴⁵ to Asp¹⁴⁵ substitution. In a specific embodiment, the degenerative disease is selected from the group consisting of multiple sclerosis, muscular dystrophy, Alzheimer's disease, focal lobar atrophies, including semantic dementia and dementia of frontal type, subcortical dementia, including progressive supranuclear palsy, Huntington's disease and Parkinson's disease, lumbar degenerative disk disease, amyotrophic lateral sclerosis, degenerative joint disease, arthritis, Creutzfeldt-Jakob disease, degenerative valve disease, retinal degenerative disease, Sorsby's fundus dystrophy, and macular degeneration.

In an additional object of the present invention, there is a method of using a composition of p21^{Cip1/WAF1} polypeptide comprising a Thr¹⁴⁵to Asp¹⁴⁵ substitution, wherein the p21^{Cip1/WAF1} polypeptide composition is dispersed in a pharmacologically acceptable excipient, and wherein the composition is administered to an animal having a degenerative disease.

In another object of the present invention, there is a method of obtaining a nuclear-retained polypeptide of p21^{Cip1/WAF1} which remains in the nucleus following activation of Akt.

In an additional object of the present invention, there is a method of obtaining a nuclear-retained polypeptide of p21^{Cip1/WAF1} which remains in the nucleus following activation of Akt, comprising obtaining a polynucleotide which encodes a p21^{Cip1/WAF1} polypeptide; altering said polynucleotide to effect a modification of the polypeptide; and administering said polypeptide to a cell having said nucleus, wherein when said modified polypeptide remains in the nucleus following activation of Akt, said polypeptide is a nucleus-retained polypeptide of p21^{Cip1/WAF1} In a specific embodiment, the modification is an amino acid substitution at Thr¹⁴⁵. In another specific embodiment, the method further comprises placing the polypeptide in a pharmacologically acceptable excipient. In another specific embodiment, the method further comprises using the polypeptide in the pharmacologically acceptable excipient to treat an animal. In an additional specific embodiment, the animal has a proliferative cell disorder. In another specific embodiment, the proliferative cell disorder is cancer. In a further specific embodiment, the cancer is breast cancer. In an additional specific embodiment, the animal is a human.

In an additional object of the present invention, there is a method of obtaining a nucleus-retained polypeptide of p21^{Cip1/WAF1} which remains in the nucleus of a cell following activation of Akt, comprising the steps of obtaining a p21^{Cip1/WAF1} polypeptide; modifying said p21^{Cip1/WAF1} polypeptide at amino acid position 145, wherein said modification results in an inability of amino acid to be phosphorylated; and administering said polypeptide to a cell having said nucleus, wherein when said modified polypeptide remains in the nucleus following activation of Akt, said polypeptide is a nucleus-retained polypeptide of p21^{Cip1/WAF1}. In a specific embodiment, the modification is an amino acid substitution at Thr¹⁴⁵. In another specific embodiment, the method further comprises placing the polypeptide in a pharmacologically acceptable excipient. In an additional specific embodiment, the method further comprises using the polypeptide in the pharmacologically acceptable excipient to treat an animal. In a specific embodiment, the animal has a proliferative cell disorder. In a further specific embodiment, the proliferative cell disorder is cancer. In a further specific embodiment, the cancer is breast cancer. In an additional specific embodiment, the animal is a human.

In another object of the present invention, there is a method of identifying a p21^{Cip1/WAF1} polypeptide which accumulates in the nucleus of a cell following activation by Akt, comprising altering the polypeptide; and assaying the polypeptide for nuclear accumulation in the cell under conditions wherein an unmodified p21^{Cip1/WAF1} polypeptide translocalizes from the nucleus to the cytoplasm of the cell.

In an additional object of the present invention, there is a method of treating a cell comprising contacting the cell with a p21^{Cip1/WAF1} polypeptide, wherein the polypeptide comprises a substitution at Thr¹⁴⁵. In a specific embodiment, the cell is a human cell. In another specific embodiment, the cell is comprised in an animal. In a further specific embodiment, the animal is a human. In a further specific embodiment, the human has a proliferative cell disorder. In an additional specific embodiment, the proliferative cell disorder is cancer. In a further specific embodiment, the cancer is breast cancer. In another specific embodiment, the proliferative cell disorder is restenosis.

In another object of the present invention, there is a method of inhibiting angiogenesis in an individual comprising the step of administering to said individual a p21^{Cip1/WAF1} polypeptide having an amino acid substitution at Thr¹⁴⁵. In a specific embodiment, the substitution is a Thr¹⁴⁵ to Ala¹⁴⁵ substitution.

### BRIEF DESCRIPTION OF THE FIGURES

The following drawings form part of the present specification and are included to further demonstrate certain aspects of the present invention. The invention may be better understood by reference to one or more of these drawings in combination with the detailed description of specific embodiments presented herein.

FIG. 1 shows that the Akt pathway was required for HER-2/neu-mediated cell proliferation. (A) Blocking the Akt pathway reduces the growth of HER-2/neu 3T3 cells. The cells (3×10³) were seeded in 96-well plates and grown in Dulbecco's modified Eagle's medium/F12 medium plus 1% fetal bovine serum. The growth rate was monitored by the MTT assay. The results are presented as the average ±SE of three independent experiments performed in quadruplicate. (B) Cells (3×10³) were grown as described in (A) with 1 μCi of [³H]thymidine for 12 hr. The cell replication rate was determined by measuring [³H]thymidine incorporation. The results are presented as the average ±SE of three independent experiments performed in quadruplicate. (C) Blocking the Akt pathway did not significantly induce apoptosis in HER-2/neu 3T3 cells. The cells were grown in 1% serum, and the percentage of cells in apoptosis was measured by fluorescence-activated cell sorting (FACS) as described previously (Zhou et al., 2000). The results are presented as the average ±SE of three independent experiments. (D) Blocking the Akt pathway also reduced cell growth in MDA-MB453 cells. The cells were grown in six-well plates as described in (A), and their growth rates were measured by cell counting with a Coulter Counter. The results are presented as the average ±SE of three independent experiments. (E) The DNA synthesis rate of each of the cell lines in (D) was determined as described in (B). (F) Over-expression of HER-2/neu induced the expression of p21^{Cip1/WAF1}. Lysates (50 μg) from each cell lines were analyzed by 12% SDS-PAGE. After the protein was transferred to a nitrocellulose membrane, the expression of p21^{Cip1/WAF1} was measured with a monoclonal antibody against p21^{Cip1/WAF1} (Phamingen).

FIG. 2 demonstrates that threonine 145 of p21^{Cip1/WAF1} was phosphorylated in vivo. (A) Endogenous p21^{Cip1/WAF1} was phosphorylated in vivo by insulin. MDA-MB453 cells were incubated with 1.5 mCi/ml [³²P]orthophosphate for 3.5 hours followed by 30 min of insulin stimulation with or without PI-3K inhibitor LY294002. The cells were then lysed, and the endogenous p21^{Cip1/WAF1} was immunoprecipitated with specific antibody and subjected to two-dimensional phosphopeptide analysis as described in the Examples. The arrow indicates the spot induced by insulin and inhibited by PI-3K inhibitor. (B) Tyrosine residues in p21^{Cip1/WAF1} were not phosphorylated. MDA-MB453 cells were treated as described above, and one third of the lysate was analyzed by western blotting for activation of Akt and tyrosine phosphorylation of HER-2/neu (2-sec exposure with anti-phosphotyrosine antibody clone 4G10 from Upstate Biotech.). The remaining lysate was subjected to immunoprecipitation with p21^{Cip1/WAF1} specific antibody, and western blotting with specific antibodies against p21^{Cip1/WAF1} and phosphotyrosine (20-min exposure with clone 4G10). (C) Threonine 145 of p21^{Cip1/WAF1} was phosphorylated. MDA-MB453 cells were treated as described in panel A, and p21^{Cip1/WAF1} was immuoprecipitated and subjected to trypsin digestion, and amino acid sequencing using Edman degradation. The radioisotope released from each cycle of Edman degradation was detected with a Cerenkov counter. The background counts, which usually ranged from 50 to 60 cpm, were subtracted from each measurement.

FIG. 3 shows that Akt interacted with p21^{Cip1/WAF1} and phosphorylated it at threonine 145. (A) The consensus Akt phosphorylation motif is highlighted. Sequence comparison of p21^{Cip1/WAF1} and other known Akt substrates is shown. (B) Immunoprecipitation of endogenous Akt and detection of endogenous p21^{Cip1/WAF1}. Endogenous Akt was immunoprecipitated from 1000 μg of MDA-MB453 cell lysate with Akt specific antibody or control IgG. After transfer to a nitrocellular membrane, endogenous p21^{Cip1/WAF1} was detected with p21^{Cip1/WAF1} specific antibody. (C) Immunoprecipitation of p21^{Cip1/WAF1} and detection of Akt. HA-tagged DN-Akt or CA-Akt (10 μg) and flag-tagged wild-type or mutant p21^{Cip1/WAF1} (10 μg) were cotransfected into 293T cells by the calcium phosphate methods. The cells were lysed in RIPA buffer after 48 hr and p21^{Cip1/WAF1} was immunoprecipitated with anti-flag antibody. After transfer to a nitrocellular membrane, Akt was detected with HA antibody. (D) Immunoprecipitation of Akt and western blotting of p21^{Cip1/WAF1}. Akt was immunoprecipitated with an HA antibody and p21^{Cip1/WAF1} was detected with a flag antibody. (E) Akt phosphorylated p21^{Cip1/WAF1} at threonine 145. HA-tagged CA-Akt or DN-Akt (20 μg) was transiently transfected into 293T cells as described herein. After 48 hr of incubation, CA-Akt or DN-Akt was immunoprecipitated with an HA antibody and incubated with 5 μg of either GST-wild-type p21^{Cip1/WAF1} or GST-mutant p21^{Cip1/WAF1} (Ti45A) in a kinase buffer containing 5 μCi of [γ³² P]ATP for 30 min at 30° C. The kinase reaction was terminated with SDS-PAGE buffer, and the samples were assayed by autoradiography. The bottom two panels show western blots of Akt and the GST fusion protein used in the phosphorylation reaction, detected with antibodies against Akt and p21^{Cip1/WAF1}. (F) CA-Akt or DN-Akt (18 μg) and flag-tagged wild-type or mutant p21^{Cip1/WAF1} (T145A) (2 μg) were cotransfected into 293T cells. After 48 hr, the cells were labeled with 1 mCi/ml [³²P]-orthophosphate for 3 hr. The p21^{Cip1/WAF1} was immunoprecipitaed from the lysate and analyzed by either autoradiography to detect phosphorylation of p21^{Cip1/WAF1} in vivo (upper panel) or western blotting as a control to measure p21^{Cip1/WAF1} protein level (bottom panel). The histogram shows the amount of labeled p21^{Cip1/WAF1} relative to the amount of immunoprecipitated p21^{Cip1/WAF1} on the western blots.

FIG. 4 demonstrates that Akt affected the cellular localization of p21^{Cip1/WAF1}. A 9:1 ratio of CA-Akt or DN-Akt (9 μg) and wild-type or mutant p21^{Cip1/WAF1} (1 μg) were cotransfected into p21^{-/-} MEF cells. After 36 hr of incubation, the cells were trypsinized and plated into chamber slides for another 12 hr. After fixation, the cellular localization of p21^{Cip1/WAF1} was detected by using a monoclonal antibody against human p21^{Cip1/WAF1}. After extensive washing in phosphate-buffered saline, the samples were further incubated with Texas Red-conjugated goat anti-mouse IgG plus dapi and examined under a fluorescent microscope (Zeiss). When wild-type p21^{Cip1/WAF1} was cotransfected with CA-Akt, approximately 75% of p21^{Cip1/WAF1} were found localized in the cytoplasm. However, when p21^{Cip1/WAF1} was cotransfected with DN-Akt, approximately 90% of wild-type p21^{Cip1/WAF1} were detected in the nucleus. When T145A mutant was cotransfected with CA-Akt or DN-Akt, approximately 80% and 90% of T145A were found both localized in the nucleus. When T145D mutant was cotransfected with CA-Akt or DN-Akt, approximately 72% and 78% of T145D were found both localized in the cytoplasm.

FIG. 5 shows cellular localization of endogenous p21^{Cip1/WAF1} (A) Cellular fractionation was performed to determine the cellular localization of p21^{Cip1/WAF1} in HER-2/neu 3T3 cells, MDA-MB453 cells, and their DN-Akt transfectants as described in the Examples. Equal amounts (40 ug) of cytoplasmic fraction (C) and nuclear fraction (N) from each samples were analyzed by 12% SDS-PAGE. Actin and PCNA were used as markers of the cytoplasmic and nuclear fractions, respectively. In each cellular fractionation, there was 10 times as much protein in the cytoplasm as in the nucleus. (B) Cellular location of endogenous p21^{Cip1/WAF1} was regulated by the activation of Akt. MDA-MB453 cells were stimulated with insulin with or without PI-3K inhibitor LY294002. Cellular fractionation was performed as described in the Examples. Equal amounts of cytoplasmic and nuclear fraction from each sample were analyzed by western blotting.

FIG. 6 demonstrates HER-2/neu activateed Akt and induced cytoplasmic localization of p21^{Cip1/WAF1} in breast tumor tissues. Tissue sections from the HER-2/neu-positive adenocarcinomas (a-c) and HER-2/neu-negative adenocarcinomas (d-f) were stained with antibodies specific to HER-2/neu (a and d), phosphorylated Akt (b and e), p21^{Cip1/WAF1} (c and f), or normal rabbit serum. The immunostaining was visualized with peroxidase-conjugated secondary antibody. The black and white arrows indicate the cytoplasm and nucleus localization of p21^{Cip1/WAF1} in panel c and f, respectively.

FIG. 7 shows p21^{Cip1/WAF1} (T145D) reduced its inhibition activity whereas p21^{Cip1/WAF1} (Ti45A) retained its inhibition activity independent of Akt. (A) The colony-formation assay was used to measure the inhibition activity of p21^{Cip1/WAF1} and its mutant. Wild-type or mutant p21^{Cip1/WAF1} (T145A or T145D) or the vector pcDNA3 (2 ug of each) was transfected into NIH3T3, HER-2/neu 3T3, and DN-Akt 3T3 cells. The number of colonies from each transfectant was determined by using crystal violet staining. The percentages of colonies from wild-type and mutant p21^{Cip1/WAF1} were calculated by defining the number obtained from vector transfection alone as 100%. The results are presented as the average ±SE of four independent experiments. (B) The Brdu incorporation assay was also used to measure the inhibition activity of p21^{Cip1/WAF1} and its mutant. Vector containing membrane-bound GFP (1 μg) and wild-type or mutant p21^{Cip1/WAF1} (T145A or T145D) or vector pcDNA3 (9 μg of each) were cotransfected into NIH3T3, HER-2/neu 3T3, and DN-Akt 3T3 cells by using liposome. After 48 hours of incubation, the cells were labeled with Brdu for 1 hour and then fixed with 70% ethanol. The cells were then stained with anti-Brdu antibody and incubated with fluorescent-conjugated secondary antibody. After extensive washing, the cells were sorted for GFP, and the incorporation of Brdu was measured by using FACS analysis. The percentage of Brdu incorporation for wild-type and mutant p21^{Cip1/WAF1} was calculated by defining the number obtained from vector (pcDNA3) alone as 100%. The results are presented as the average I SE of two separate experiments.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### DETAILED DESCRIPTION OF THE INVENTION

As used herein the specification, “a” or “an” may mean one or more. As used herein in the claim(s), when used in conjunction with the word “comprising”, the words “a” or “an” may mean one or more than one. As used herein “another” may mean at least a second or more. I. The Present Invention

Amplification/overexpression of HER-2/neu in cancer cells confers resistance to apoptosis and promotes cell growth. Cellular localization of p21^{Cip1/WAF1} has been proposed to be critical in either promoting cell survival or inhibiting cell growth. The conflicting dual functions of p21^{Cip1/WAF1} have made it difficult for application for therapeutic purposes. In the present invention, the inventors demonstrate that HER-2/neu-mediated cell growth required the activation of Akt, which associated with p21^{Cip1/WAF1} and phosphorylated it at threonine 145, which resulted in cytoplasmic localization of p21^{Cip1/WAF1}. Furthermore, blocking the Akt pathway with a dominant-negative mutant of Akt restored the nuclear localization and the cell growth inhibition activity of p21^{Cip1/WAF1}. Thus, HER-2/neu induces cytoplasmic localization of p21^{Cip1/WAF1} via the activation of Akt to promote cell growth, which has implications in the oncogenic activity of HER-2/neu and Akt.

Two exemplary mutants of p21^{Cip1/WAF1}, p21-T145A (threonine 145 to alanine) and p21-T145D (threonine 145 to aspartic acid), were generated. The p21-T145A mutant is primarily located in the nucleus and preferentially suppresses growth of transformed cells. Thus, the mutant p21-T145A in preferred embodiments of the present invention selectively inhibits cancer cell growth and also preferably is a more potent anti- cancer agent than the wild-type p21^{Cip1/WAF1}. In other embodiments the mutant p21-T145A inhibits other cell proliferation and is useful for the treatment of restenosis or to inhibit angiogenesis. Another mutant, p21-T145D mimics phosphorylation of p21^{Cip1/WAF1} and is therefore primarily located in the cytoplasm. In other embodiments of the present invention the cytoplasmic p21-T145D will help cells to survive and are useful in the prevent and/or treatment of degenerative disease. However, one skilled in the art following the teachings of this specification can create other exemplary mutants of p21^{Cip1/WAF1} polypeptide.

A skilled artisan recognizes that a multitude of p21^{Cip1/WAF1} nucleic acid sequences may be used in the present invention. A skilled artisan is aware of publicly available databases which provide these sequences, such as the National Center for Biotechnology Information's GenBank database or commercially available databases such as from Celera Genomics, Inc. (Rockville, Md.). Exemplary sequences include (as identified by their GenBank Accession numbers): BC001935 (SEQ ID NO: 1); BC000312 (SEQ ID NO: 2); BC000275 (SEQ ID NO: 3); XM_{—}011458 (SEQ ID NO: 4); NM_{—}007669 (SEQ ID NO: 5); U24172 (SEQ ID NO: 6); U24174 (SEQ ID NO: 7); U24173 (SEQ ID NO: 8); L41275 (SEQ ID NO: 9); L47233 (SEQ ID NO: 10); U24171 (SEQ ID NO: 11); U50603 (SEQ ID NO: 12); L25610 (SEQ ID NO: 13); NM_{—}000389 (SEQ ID NO: 14); AF076469 (SEQ ID NO: 15); BB503715 (SEQ ID NO: 16); BF149406 (SEQ ID NO: 17); BB466338 (SEQ ID NO: 18); BB422549 (SEQ ID NO: 19); BB372883 (SEQ ID NO: 20); BB261613 (SEQ ID NO: 21); BB253758 (SEQ ID NO: 22); BB249561 (SEQ ID NO: 23); BB247445 (SEQ ID NO: 24); BB212516 (SEQ ID NO: 25); BB057018 (SEQ ID NO: 26); AV216269 (SEQ ID NO: 27); AV215507 (SEQ ID NO: 28); AV214274 (SEQ ID NO: 29); AV207714 (SEQ ID NO: 30); AR035960 (SEQ ID NO: 31); AR035955 (SEQ ID NO: 32); U24170 (SEQ ID NO: 33); C79124 (SEQ ID NO: 34); C78697 (SEQ ID NO: 35); U03106 (SEQ ID NO: 36); D84650 (SEQ ID NO: 37); CO₅₉₅₅ (SEQ ID NO: 38); BC002043 (SEQ ID NO: 39); U09579 (SEQ ID NO: 40); L26165 (SEQ ID NO: 41); L47232 (SEQ ID NO: 42); S67388 (SEQ ID NO: 43); U09507 (SEQ ID NO: 44); AK007630 (SEQ ID NO: 45); AB017818 (SEQ ID NO: 46); AB017817 9SEQ ID NO: 47); and U34594 (SEQ ID NO: 48).

Examples of p21^{Cip1/WAF1} polypeptides which may be useful in the present invention are as follows: AAH01935.1 (SEQ ID NO: 49); AAH00312.1 (SEQ ID NO: 50); AAH00275.1 (SEQ ID NO: 51); XP_{—}011458.1 (SEQ ID NO: 52); NP_{—}031695.1 (SEQ ID NO: 53); AAC52221.1 (SEQ ID NO: 54); AAC52220.1 (SEQ ID NO: 55); AAC42084.1 (SEQ ID NO: 56); AAB59560.1 (SEQ ID NO: 57); AAB59559.1 (SEQ ID NO: 58); AAA16109 (SEQ ID NO: 59); NP_{—}000380.1 (SEQ ID NO: 60); AACO_{4313.1} (SEQ ID NO: 61); BAA23168.1 (SEQ ID NO: 62); AAH02043.1 (SEQ ID NO: 63); AAA85641.1 (SEQ ID NO: 64); AAA19811.1 (SEQ ID NO: 65); AAA16109.1 (SEQ ID NO: 66); AAC27627.1 (SEQ ID NO: 67); AAB59560.1 (SEQ ID NO: 68); AAB29246.1 (SEQ ID NO: 69); BAB25148.1 (SEQ ID NO: 70); BAA82342.1 (SEQ ID NO: 71); BAA82341 (SEQ ID NO: 72); and AAA91031.1 (SEQ ID NO: 73).

A skilled artisan recognizes that the two exemplary mutants of p21^{Cip1/WAF1}, p21-T145A (threonine 145 to alanine) (SEQ ID NO:77) and p21-T145D (threonine 145 to aspartic acid) (SEQ ID NO:78) may be generated by a variety of means. In a specific embodiment, a nucleic acid sequence as set forth in any of SEQ ID NO:1 through SEQ ID NO:48 is mutated at the codon which encodes the threonine at residue 145 to encode an alanine or aspartic acid, respectively. Table 1 presents codons for all standard amino acids, and a skilled artisan would be well aware how to manipulate a starting nucleic acid to generate a desired mutation using standard site-directed mutagenesis techniques, for example.

In an embodiment of the present invention, the p21^{Cip1/WAF1} gene product is phosphorylated following HER-2/neu-mediated activation of Akt. In particular, the inventors have shown herein that the T145 residue, as a result of an inability to be phosphorylated, renders the p21^{Cip1/WAF1} gene product unable to be translocated from the nucleus to the cytoplasm. Retention in the nucleus of the cell in question is associated with inhibition of cell growth. A skilled artisan recognizes that the T145 residue may be altered to prohibit phosphorylation. For example, the T145 amino acid residue may be changed by altering the nucleic acid codon which encodes it, such as by site-directed mutagenesis. Alternatively, the T145 amino acid may be blocked with at least one compound which prevents phosphorylation, for example with blocking agents such as carbodiamide or by acetylation of the residue with acetylchloride in trifluoroacetic acid.

In an alternative embodiment of the present invention, a p21^{Cip1/WAF1} polypeptide is modified so that nuclear accumulation in a cell occurs in lieu of translocation to the cytoplasm following activation of Akt. The modification may be to Thr¹⁴⁵ or to another amino acid or amino acids in the polypeptide which results in similar activity to the activities of the exemplary mutants described herein. It is contemplated that the modification to the polypeptide may result in substantial accumulation of p21^{Cip1/WAF1} polypeptide in the nucleus, although a fraction of accumulation is also within the scope of the invention provided that it results in at least some inhibition of growth of the cell. Furthermore, it is contemplated herein that Akt may be activated through a variety of mechanisms, although in a preferred embodiment Akt is activated via the HER-2/neu pathway.

In the scope of the present invention, a p21^{Cip1/WAF1} polypeptide comprises a substitution which prevents nucleus-to-cytoplasm translocation in a cell following activation of Akt. A skilled artisan recognizes that the substitution may prevent phosphorylation of the p21^{Cip1/WAF1} polypeptide under conditions which would result in phosphorylation of an unsubstituted p21^{Cip1/WAF1} polypeptide, and furthermore would know methods standard in the art to determine these conditions. The nuclear accumulation of p21^{Cip1/WAF1} polypeptide following activation of Akt preferably occurs under conditions in which an unsubstituted p21^{Cip1/WAF1} polypeptide would translocate from the nucleus to the cytoplasm of the cell.

In embodiments of the present invention, there are methods of preventing cytoplasmic translocation of p21^{Cip1/WAF1} polypeptide from a nucleus of a cell following Akt activation comprising administering to the cell a p21^{Cip1/WAF1} polypeptide having an amino acid substitution at Thr¹⁴⁵, or, alternatively, any modification to the polypeptide that results in the nuclear accumulation. In other embodiments of the present invention, there are methods of preventing growth of a cell in an individual comprising administering to the individual a p21^{Cip1/WAF1} polypeptide having an amino acid substitution at Thr¹⁴⁵, or, alternatively, any modification to the polypeptide that results in the nuclear accumulation. In specific embodiments, the polypeptide is administered in a liposome and/or the polypeptide further comprises a protein transduction domain (Schwarze et al., 1999). In alternative embodiments, p21^{Cip1/WAF1} is administered as a polynucleotide, wherein the polynucleotide comprises the alteration which effects modification at the amino acid level, such as is generated by site-directed mutagenesis. The modified p21^{Cip1/WAF1} polynucleotide is administered in a vector such as a plasmid, retroviral vector, adenoviral vector, adeno-associated viral vector, liposome, or a combination thereof.

In other embodiments of the present invention, the p21^{Cip1/WAF1} polypeptide comprises an amino acid substitution which mimics phosphorylation of p21^{Cip1/WAF1} and, for example, can be at T145. In a specific example, the amino acid substitution is T145D. Of course, other substitutions at this location or substitutions at other locations having the same effect can be derived by one skilled in the art. Such mutations will have cytoplasmic localization for prevention or treatment of degenerative disease.

In other embodiments of the present invention, the p21^{Cip1/WAF1} polypeptide comprises an amino acid substitution of p21^{Cip1/WAF1} which remains nuclearly localized following activation of Akt, and, for example, can be at T145. In a specific example, the amino acid substitution is T145A. Of course, other substitutions at this location or substitutions at other locations having the same effect can be derived by one skilled in the art. Such mutations will have nuclear retention following activation of Akt for prevention or treatment of degenerative disease.

Within the scope of the invention are methods of obtaining a nuclear-retained p21^{Cip1/WAF1} polypeptide which remains in the nucleus following activation of Akt, comprising obtaining a polynucleotide which encodes the p21^{Cip1/WAF1} polypeptide, altering the polynucleotide to effect a modification of the polypeptide, such as amino acid substitution at Thr¹⁴⁵, and administering the polypeptide to a cell having a nucleus, wherein when the modified polypeptide remains in the nucleus following activation of Akt, the polypeptide is a nucleus-retained polypeptide of p21^{Cip1/WAF1}. Alternatively, a p21^{Cip1/WAF1} polypeptide is obtained and modified at Thr¹⁴⁵ wherein the modification results in inability of the amino acid to be phosphorylated, and the polypeptide is administered to a cell having the nucleus, wherein when the modified peptide remains in the nucleus following activation of Akt, the polypeptide is a nucleus-retained polypeptide of p21^{Cip1/WAF1}.

Other embodiments of the present invention include a method of identifying a p21^{Cip1/WAF1} polypeptide which accumulates in the nucleus of a cell following activation by Akt comprising altering the polypeptide and assaying the localization of the polypeptide for nuclear accumulation in the cell under conditions wherein an unmodified p21^{Cip1/WAF1} polypeptide translocalizes from the nucleus to the cytoplasm of the cell. A skilled artisan recognizes that there are a variety of ways well known in the art to alter the polypeptide.

There are also embodiments of the present invention wherein there are methods of treating a cell comprising contacting the cell with a p21^{Cip1/WAF1} polypeptide, wherein the polypeptide comprises a substitution at Thr¹⁴⁵. In specific embodiments, the cell is a human cell, the cell is comprised in an animal, and/or the animal is human.

It is contemplated herein that the compositions of the present invention preferably have an activity different from a native p21^{Cip1/WAF1} polypeptide in the cell, and that the scope of the present invention is directed to a change in the native p21^{Cip1/WAF1} polypeptide for use in a manner different from the wildtype p21^{Cip1/WAF1} polypeptide.

II. p21^{Cip1/WAF1} and Subcellular Localization

A skilled artisan recognizes that the subcellular localization of p21^{Cip1/WAF1} is paramount to its activity, and the inventors have provided in the Examples herein p21^{Cip1/WAF1} compositions that have modifications that affect their subcellular localization. The dual roles of p21^{Cip1/WAF1} in relation to effects on cell proliferation and death are discussed in the art in reviews such as Porter (1999), incorporated by reference herein in its entirety.

The abnormal activation of cyclin-dependent kinases (cdc2 and CDKs 1, 2 and 3) is strongly associated with apoptosis in a variety of cell-death mechanisms. One such mechanism for the activation of nuclear cyclin-CDK2 complexes in endothelial cells undergoing apoptosis involves the specific cleavage of the CDK inhibitors p21^{Cip1/WAF1} and p21^{Cip1/WAF1} by caspase-3 (Levkau et al., 1998). A p21^{Cip1/WAF1} lacking its C-terminal nuclear-localization signal consequently exits the nucleus, which relieves the inhibition of CDK2. Apoptosis is suppressed in part by blocking CDK2 activation or cleavage of p21^{Cip1/WAF1}, which indicates that apoptosis is associated with the activation of CDK2 following nucleus-to-cytoplasm transport of cleaved p21^{Cip1/WAF1}.

Nuclear-localized p21^{Cip1/WAF1} furthermore becomes cytoplasmic following differentiation into monocytes of U937 cells, but, as opposed to endothelial cells (Levkau et al., 1998), this translocation mechanism is associated with resistance to various apoptotic stimuli (Asada et al., 1999). Cytoplasmic p21^{Cip1/WAF1} forms a complex with apoptosis signal-regulating kinase 1 (ASK1) that in monocytes inhibits the stress-induced mitogen-activated protein (MAP) kinase cascade (Asada et al., 1999). Overexpression of a p21^{Cip1/WAF1} mutant lacking the nuclear-localization signal also results in a cytoplasmic complex with ASK1 and resistance to apoptosis in U937 cells (Asada et al., 1999) Monocytes destroy intracellular pathogens and extracellular non-self targets through the production of hydrogen peroxide and reactive oxygen species. Therefore, the strong protective effect of cytoplasmic p21^{Cip1/WAF1} might be due to inhibition of the stress-induced MAP kinase pathway (Jarpe et al., 1998), thereby ensuring survival of monocytes in the presence of the high levels of hydrogen peroxide and reactive oxygen species that are needed to kill their targets.

Thus, there are disparate functions of cytoplasmic full-length and truncated p21^{Cip1/WAF1} in apoptosis (Levkau et al., 1998; Asada et al., 1999), discuss the mechanisms, specifically regarding translocation of p21^{Cip1/WAF1} to the cytoplasm, underlying the ability of p21^{Cip1/WAF1} either to sensitize or to protect difference cell types from apoptosis (Levkau et al., 1998; Asada et al., 1999; Jiang and Porter, 1998; and references therein).

III. Definitions and Techniques Affecting p21^{Cip1/WAF1} Gene Products and Genes

A. p21^{Cip1/WAF1} Gene Products and Genes

As used herein, the terms “p21^{Cip1/WAF1} gene product” and “p21^{Cip1/WAF1}” refer to proteins having amino acid sequences which are substantially identical to the native p21^{Cip1/WAF1} or which are biologically active in that they are capable of cross-reacting with anti-p21^{Cip1/WAF1} antibody raised against p21^{Cip1/WAF1}. “p21^{Cip1/WAF1} gene product” and “p21^{Cip1/WAF1}” refer to proteins having amino acid sequences which are substantially identical to the native p21^{Cip1/WAF1} amino acid sequence and which are biologically active in that they are capable of binding to ETS binding sites or cross-reacting with anti-p21^{Cip1/WAF1} antibody raised against p21^{Cip1/WAF1}. Such sequences are disclosed, for example, in Macleod et al., (1992). The term “p21^{Cip1/WAF1} gene product” also includes analogs of p21^{Cip1/WAF1} molecules which exhibit at least some biological activity in common with native p21^{Cip1/WAF1}. Furthermore, those skilled in the art of mutagenesis will appreciate that other analogs, as yet undisclosed or undiscovered, may be used to construct p21^{Cip1/WAF1} analogs.

The term “mutant form of p21^{Cip1/WAF1}” refers to any DNA sequence that is substantially identical to a DNA sequence encoding a p21^{Cip1/WAF1} gene product as defined above. The term also refers to RNA, or antisense sequences compatible with such DNA sequences. A “p21^{Cip1/WAF1} gene” may also comprise any combination of associated control sequences.

The term “substantially identical”, when used to define either a p21^{Cip1/WAF1} amino acid sequence or p21^{Cip1/WAF1} gene nucleic acid sequence, means that a particular subject sequence, for example, a mutant sequence, varies from the sequence of natural p21^{Cip1/WAF1} by one or more substitutions, deletions, or additions, the net effect of which is to retain at least some biological activity of the p21^{Cip1/WAF1} protein. Alternatively, DNA analog sequences are “substantially identical” to specific DNA sequences disclosed herein if: (a) the DNA analog sequence is derived from coding regions of the natural p21^{Cip1/WAF1} gene; or (b) the DNA analog sequence is capable of hybridization of DNA sequences of (a) under moderately stringent conditions and which encode biologically active p21^{Cip1/WAF1}; or (c) DNA sequences which are degenerative as a result of the genetic code to the DNA analog sequences defined in (a) or (b). Substantially identical analog proteins will be greater than about 80% similar to the corresponding sequence of the native protein. Sequences having lesser degrees of similarity but comparable biological activity are considered to be equivalents. In determining nucleic acid sequences, all subject nucleic acid sequences capable of encoding substantially similar amino acid sequences are considered to be substantially similar to a reference nucleic acid sequence, regardless of differences in codon sequence.

B. Percent Similarity

Percent similarity may be determined, for example, by comparing sequence information using the GAP computer program, available from the University of Wisconsin Geneticist Computer Group. The GAP program utilizes the alignment method of Needleman et al., 1970, as revised by Smith et al., 1981. Briefly, the GAP program defines similarity as the number of aligned symbols (i.e. nucleotides or amino acids) which are similar, divided by the total number of symbols in the shorter of the two sequences. The preferred default parameters for the GAP program include (1) a unitary comparison matrix (containing a value of 1 for identities and 0 for non-identities) of nucleotides and the weighted comparison matrix of Gribskov et al., 1986, as described by Schwartz et al., 1979; (2) a penalty of 3.0 for each gap and an additional 0.01 penalty for each symbol and each gap; and (3) no penalty for end gaps.

C. Nucleic Acid Sequences

In certain embodiments, the invention concerns the use of p21^{Cip1/WAF1} nucleic acids, genes and gene products, such as the p21^{Cip1/WAF1} that includes a sequence which is essentially that of the known p21^{Cip1/WAF1} gene, or the corresponding protein. The term “a sequence essentially as p21^{Cip1/WAF1}” means that the sequence substantially corresponds to a portion of the p21^{Cip1/WAF1} gene and has relatively few bases or amino acids (whether DNA or protein) which are not identical to those of p21^{Cip1/WAF1} (or a biologically functional equivalent thereof, when referring to proteins). The term “biologically functional equivalent” is well understood in the art and is further defined in detail herein. Accordingly, sequences which have between about 70% and about 80%; or more preferably, between about 81% and about 90%; or even more preferably, between about 91% and about 99%; of amino acids which are identical or functionally equivalent to the amino acids of p21 will be sequences which are “essentially the same”.

p21 nucleic acids which have functionally equivalent codons are also covered by the invention. The term “functionally equivalent codon” is used herein to refer to codons that encode the same amino acid, such as the six codons for arginine or serine, and also refers to codons that encode biologically equivalent amino acids (Table 1).

TABLE 1
FUNCTIONALLY EQUIVALENT CODONS

| Amino Acids | Codons | | |
|---|---|---|---|
| Alanine | Ala | A | GCA GCC GCG GCU |
| Cysteine | Cys | C | UGC UGU |
| Aspartic Acid | Asp | D | GAC GAU |
| Glutamic Acid | Glu | E | GAA GAG |
| Phenylalanine | Phe | F | UUC UUU |
| Glycine | Gly | G | GGA GGC GGG GGU |
| Histidine | His | H | CAC CAU |
| Isoleucine | Ile | I | AUA AUC AUU |
| Lysine | Lys | K | AAA AAG |
| Leucine | Leu | L | UUA UUG CUA CUC CUG CUU |
| Methionine | Met | M | AUG |
| Asparagine | Asn | N | AAC AAU |
| Proline | Pro | P | CCA CCC CCU |
| Glutamine | Gln | Q | CAA CAG |
| Arginine | Arg | R | AGA AGG CGA CGC CGG CGU |
| Serine | Ser | S | AGC AGU UCA UCC UCG UCU |
| Threonine | Thr | T | ACA ACC ACG ACU |
| Valine | Val | V | GUA GUC GUG GUU |
| Tryptophan | Trp | W | UGG |
| Tyrosine | Tyr | Y | UAC UAU |

It will also be understood that amino acid and nucleic acid sequences may include additional residues, such as additional N- or C-terminal amino acids or 5′ or 3′ sequences, and yet still be essentially as set forth in one of the sequences disclosed herein, so long as the sequence meets the criteria set forth above, including the maintenance of biological protein activity where protein expression is concerned. The addition of terminal sequences particularly applies to nucleic acid sequences which may, for example, include various non-coding sequences flanking either of the 5′ or 3′ portions of the coding region or may include various internal sequences, i.e., introns, which are known to occur within genes.

The present invention also encompasses the use of DNA segments which are complementary, or essentially complementary, to the sequences set forth in the specification. Nucleic acid sequences which are “complementary” are those which are capable of base-pairing according to the standard Watson-Crick complementarity rules. As used herein, the term “complementary sequences” means nucleic acid sequences which are substantially complementary, as may be assessed by the same nucleotide comparison set forth above, or as defined as being capable of hybridizing to the nucleic acid segment in question under relatively stringent conditions such as those described herein.

D. Biologically Functional Equivalents

As mentioned above, modification and changes may be made in the structure of p21^{Cip1/WAF1} and still obtain a molecule having like or otherwise desirable characteristics. For example, certain amino acids may be substituted for other amino acids in a protein structure without appreciable loss of interactive binding capacity with structures such as, for example, the neu-gene. Since it is the interactive capacity and nature of a protein that defines that protein's biological functional activity, certain amino acid sequence substitutions can be made in a protein sequence (or, of course, its underlying DNA coding sequence) and nevertheless obtain a protein with like or even countervailing properties (e.g., antagonistic v. agonistic). It is thus contemplated by the inventors that various changes may be made in the sequence of the p21 proteins or peptides (or underlying DNA) without appreciable loss of their desired biological utility or activity.

It is also well understood by the skilled artisan that, inherent in the definition of a biologically functional equivalent protein or peptide, is the concept that there is a limit to the number of changes that may be made within a defined portion of the molecule and still result in a molecule with an acceptable level of equivalent biological activity. Biologically functional equivalent peptides are thus defined herein as those peptides in which certain, not most or all, of the amino acids may be substituted. Of course, a plurality of distinct proteins/peptides with different substitutions may easily be made and used in accordance with the invention.

It is also well understood that where certain residues are shown to be particularly important to the biological or structural properties of a protein or peptide, e.g., residues in active sites, such residues may not generally be exchanged.

Amino acid substitutions, such as those which might be employed in modifying PBA3 are generally based on the relative similarity of the amino acid side-chain substituents, for example, their hydrophobicity, hydrophilicity, charge, size, and the like. An analysis of the size, shape and type of the amino acid side-chain substituents reveals that arginine, lysine and histidine are all positively charged residues; that alanine, glycine and serine are all a similar size; and that phenylalanine, tryptophan and tyrosine all have a generally similar shape. Therefore, based upon these considerations, arginine, lysine and histidine; alanine, glycine and serine; and phenylalanine, tryptophan and tyrosine; are defined herein as biologically functional equivalents.

In making such changes, the hydropathic index of amino acids may be considered. Each amino acid has been assigned a hydropathic index on the basis of their hydrophobicity and charge characteristics, these are: isoleucine (+4.5); valine (+4.2); leucine (+3.8); phenylalanine (+2.8); cysteine/cystine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

The importance of the hydropathic amino acid index in conferring interactive biological function on a protein is generally understood in the art (Kyte and Doolittle, 1982, incorporated herein by reference). It is known that certain amino acids may be substituted for other amino acids having a similar hydropathic index or score and still retain a similar biological activity. In making changes based upon the hydropathic index, the substitution of amino acids whose hydropathic indices are within ^{±}2 is preferred, those which are within ^{±}1 are particularly preferred, and those within ^{±}0.5 are even more particularly preferred.

It is also understood in the art that the substitution of like amino acids can be made effectively on the basis of hydrophilicity. U.S. Pat. No. 4,554,101, incorporated herein by reference, states that the greatest local average hydrophilicity of a protein, as governed by the hydrophilicity of its adjacent amino acids, correlates with its immunogenicity and antigenicity, i.e. with a biological property of the protein. It is understood that an amino acid can be substituted for another having a similar hydrophilicity value and still obtain a biologically equivalent protein.

As detailed in U.S. Pat. No. 4,554,101, the following hydrophilicity values have been assigned to amino acid residues: arginine (+3.0); lysine (+3.0); aspartate (+3.0.+−0.1); glutamate (+3.0.+−0.1); serine (+0.3); asparagine (+0.2); glutamine (+0.2); glycine (0); threonine (−0.4); proline (−0.5.+−0.1); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4).

In making changes based upon similar hydrophilicity values, the substitution of amino acids whose hydrophilicity values are within ±2 is preferred, those which are within ±1 are particularly preferred, and those within ±0.5 are even more particularly preferred.

While discussion has focused on functionally equivalent polypeptides arising from amino acid changes, it will be appreciated that these changes may be effected by alteration of the encoding DNA; taking into consideration also that the genetic code is degenerate and that two or more codons may code for the same amino acid.

IV. HER2/neu-Mediated Activation of Cellular Proliferation

It is well established that a variety of cancers are caused, at least in part, by genetic abnormalities that result in either the over-expression of one or more genes, or the expression of an abnormal or mutant gene or genes. For example, in many cases, the expression of oncogenes is known to result in the development of cancer. “Oncogenes” are genetically altered genes whose mutated expression product somehow disrupts normal cellular function or control (Spandidos et al., 1989).

Most oncogenes studied to date have been found to be “activated” as the result of a mutation, often a point mutation, in the coding region of a normal cellular gene, i.e., a “proto-oncogene”, that results in amino acid substitutions in the expressed protein product. This altered expression product exhibits an abnormal biological function that takes part in the neoplastic process (Travali et al., 1990). The underlying mutations can arise by various means, such as by chemical mutagenesis or ionizing radiation. A number of oncogenes and oncogene families, including ras, myc, neu, raf erb, src, fins, jun and abl, have now been identified and characterized to varying degrees (Travali et al., 1990; Bishop, 1987).

The neu gene (also known as HER-2/neu or c-erb-2) encodes a 185-kDa transmembrane tyrosine kinase (p185^{neu}) with homology to epidermal growth factor receptor (Hung et al., 1986; Coussens et al., 1985; Schechter et al., 1984; Sanba et al., 1985; Yamamoto et al., 1986). Enhanced expression of neu is known to be involved in many human cancers, including NSCLC and has been shown to correlate with poor patient survival in NSCLC (Kern et al., 1990; Schneider et al., 1981; Weiner et al., 1990). Cellular and animal studies have shown that an increase in neu tyrosine kinase activity increases the expression of malignant phenotypes (Muller et al., 1988; Hudziak et al., 1987; Muthuswamy et al., 1994; Yu et al., 1991; Yu et al., 1993; Hung et al., 1989; Sistonen et al., 1989; Yu et al., 1994).

The neu oncogene, was first identified in transfection studies in which NIH3T3 cells were transfected with DNA from chemically induced rat neuroglioblastomas (Shih et al., 1981). The p185 protein encoded by neu has an extracellular, transmembrane, and intracellular domain, and therefore has a structure consistent with that of a growth factor receptor (Schechter et al., 1984). The human neu gene was first isolated due to its homology with v-erbB and EGF-r probes (Senba et al., 1985).

The neu oncogene plays an important role in carcinogenesis, for example, the gene is amplified in approximately 30% of primary breast cancer. Amplified expressions of the neu oncogene in transfected 3T3 cells induces malignant transformation. neu expression has also been detected in ovarian cancer and its overexpression results in poor prognosis. The expression of neu oncogenes in human tumor cells induce resistance to several host cytotoxic mechanisms.

Along with an increased proliferative potential, neu-mediated cancers appear to be resistant to host defense mechanisms. Studies have shown that overexpression of the neu oncogene in transfected cells results in resistance to tumor necrosis factor, a major effector molecule in macrophage-mediated tumor cell cytotoxicity.

Therefore, neu oncogene expression is correlated with the incidence of cancers of the human breast and female genital tract. Moreover, amplification/overexpression of this gene has been directly correlated with relapse and survival in human breast cancer (Slamon et al., 1987; 1989). Therefore, it is important to evolve information regarding the neu oncogene, particularly information that could be applied to reversing or suppressing the oncogenic progression that seems to be elicited by the presence or activation of this gene. Unfortunately, little has been previously known about the manner in which one may proceed to suppress the oncogenic phenotype associated with the presence of oncogenes such as the neu oncogene.

In addition, neu overexpression in NSCLC is associated with shortened survival. In vitro experimental models have provided evidence that, in the murine cell NIH 3T3, oncogenes increase drug resistance. Furthermore, Tsai et al. (1993; 1995) used a NSCLC model to demonstrate that activation of an oncogene is quantitatively associated with intrinsic chemoresistance in human malignant cells. This resistance is observed with a variety of drugs that are structurally unrelated and act on different targets and/or by different mechanisms. Thus increased expression of neu oncogene enhances chemoresistance to a wide variety of chemotherapeutic agents (Tsai, 1993) including cisplatin, doxorubicin, and VP16 (Tsai et al., 1993; Tsai et al., 1995). The association of neu overexpression in cancer cells with malignant phenotypes and chemoresistance provides a plausible interpretation for the poor clinical outcome for patients with neu overexpressing tumors.

Although breast cancer diagnosed in its earliest clinical stages (stage 0, stage 1a) is highly curable, the cure rate for more advanced stages drops precipitously, even after modem combined-modality treatments. Metastatic breast cancer responds to both chemotherapy and hormone therapy, and most patients can be palliated adequately during the 1 to 3 years of usual survival. However, metastatic breast cancer is considered incurable, as demonstrated by the relentless death rates, regardless of the treatment modality utilized. Front-line chemotherapy or hormone therapy programs for correctly selected patients produce objective responses in 50% to 70% of patients, but the median duration of response is usually less than one year. Response rates after second line treatments are considerably lower (20% to 50%), and response durations average 6 months.

Ovarian cancer is also highly curable in its earliest stages, but the overwhelming majority of patients are diagnosed in stages III and IV. Although responsive to chemotherapy, most patients with advanced ovarian cancer relapse and die of their disease. With the introduction of several neu cytotoxic agents (taxanes, vinorelbine, platinum derivatives), some responses are observed after second line therapy too, but cure in this situation remains an elusive goal.

Thus overexpression of the HER-2/neu oncogene correlates with poor survival for breast and ovarian cancer patients and induces metastatic potential and chemoresistance of human cancer cells. Repression of HER-2/neu suppresses the malignant phenotypes of HER-2/neu-overexpressing cancer cells. It suggests that HER-2/neu oncogene is an excellent target for development of novel therapeutic agent for the HER-2/neu-overexpressing cancer cells. Thus methods and compositions that repress HER-2/neu transcription in HER-2/neu-overexpressing human breast and ovarian cancer cell lines, and suppress activated neu induced transformation would be of great therapeutic value in the treatment of these diseases. Mutants of p21^{Cip1/WAF1}, as described herein, have a great potential to be used as a therapeutic strategy of, for example, HER2/neu-mediated cancer types.

In 10-20% of the HER2 overexpressing breast tumors, some gastric and virtually all HER2+ lung cancers HER2 mRNA and protein overexpression occur in the absence of increased gene copy number thus suggesting that HER2 there may be some aberration in transcriptional regulation that plays a fundamental role in the development of these diseased states.

Thus, overexpression of the HER-2/neu protooncogene is found in 20-30% of primary breast cancers and in a similar fraction of human gastric, ovarian and lung adenocarcinoma. The overexpression of this membrane growth factor receptor is associated with HER2 gene amplification, more aggressive tumor growth and a reduced patient survival. In 10-20% of the HER2 overexpressing breast tumors, some gastric and virtually all HER2+ lung cancers HER2 mRNA and protein overexpression occur in the absence of increased gene copy number thus suggesting that with HER2 there may be some aberration in transcriptional regulation that plays a fundamental role in the development of these diseased states.

V. Combination Treatments

In order to increase the effectiveness of a mutant form of p21^{Cip1/WAF1}, or expression construct coding therefore, it may be desirable to combine these compositions with other agents effective in the treatment of hyperproliferative disease, such as anti-cancer agents. An “anti-cancer” agent is capable of negatively affecting cancer in a subject, for example, by killing cancer cells, inducing apoptosis in cancer cells, reducing the growth rate of cancer cells, reducing the incidence or number of metastases, reducing tumor size, inhibiting tumor growth, reducing the blood supply to a tumor or cancer cells, promoting an immune response against cancer cells or a tumor, preventing or inhibiting the progression of cancer, or increasing the lifespan of a subject with cancer. More generally, these other compositions would be provided in a combined amount effective to kill or inhibit proliferation of the cell. This process may involve contacting the cells with the expression construct and the agent(s) or multiple factor(s) at the same time. This may be achieved by contacting the cell with a single composition or pharmacological formulation that includes both agents, or by contacting the cell with two distinct compositions or formulations, at the same time, wherein one composition includes the expression construct and the other includes the second agent(s).

Tumor cell resistance to chemotherapy and radiotherapy agents represents a major problem in clinical oncology. One goal of current cancer research is to find ways to improve the efficacy of chemo- and radiotherapy by combining it with gene therapy. For example, the herpes simplex-thymidine kinase (HS-tK) gene, when delivered to brain tumors by a retroviral vector system, successfully induced susceptibility to the antiviral agent ganciclovir (Culver et al., 1992). In the context of the present invention, it is contemplated that p21^{Cip1/WAF1} gene therapy could be used similarly in conjunction with chemotherapeutic, radiotherapeutic, or immunotherapeutic intervention, in addition to other pro-apoptotic or cell cycle regulating agents.

Alternatively, the gene therapy may precede or follow the other agent treatment by intervals ranging from minutes to weeks. In embodiments where the other agent and expression construct are applied separately to the cell, one would generally ensure that a significant period of time did not expire between the time of each delivery, such that the agent and expression construct would still be able to exert an advantageously combined effect on the cell. In such instances, it is contemplated that one may contact the cell with both modalities within about 12-24 h of each other and, more preferably, within about 6-12 h of each other. In some situations, it may be desirable to extend the time period for treatment significantly, however, where several d (2, 3, 4, 5, 6 or 7) to several wk (1, 2, 3, 4, 5, 6, 7 or 8) lapse between the respective administrations.

Various combinations may be employed, gene therapy is “A” and the secondary agent, such as radio- or chemotherapy, is “B”:

| | | | | | |
|---|---|---|---|---|---|
| A/B/A | B/A/B B/B/A | A/A/B | A/B/B B/A/A | A/B/B/B | B/A/B/B |
| B/B/B/A | B/B/A/B | A/A/B/B | A/B/A/B | A/B/B/A | B/B/A/A |
| B/A/B/A | B/A/A/B | A/A/A/B | B/A/A/A | A/B/A/A | A/A/B/A |

Administration of the therapeutic expression constructs of the present invention to a patient will follow general protocols for the administration of chemotherapeutics, taking into account the toxicity, if any, of the vector. It is expected that the treatment cycles would be repeated as necessary. It also is contemplated that various standard therapies, as well as surgical intervention, may be applied in combination with the described hyperproliferative cell therapy.

A. Chemotherapy

Cancer therapies also include a variety of combination therapies with both chemical and radiation based treatments. Combination chemotherapies include, for example, cisplatin (CDDP), carboplatin, procarbazine, mechlorethamine, cyclophosphamide, camptothecin, ifosfamide, melphalan, chlorambucil, busulfan, nitrosurea, dactinomycin, daunorubicin, doxorubicin, bleomycin, plicomycin, mitomycin, etoposide (VP16), tamoxifen, raloxifene, estrogen receptor binding agents, taxol, gemcitabien, navelbine, famesyl-protein tansferase inhibitors, transplatinum, 5-fluorouracil, vincristin, vinblastin and methotrexate, or any analog or derivative variant of the foregoing.

B. Radiotherapy

Other factors that cause DNA damage and have been used extensively include what are commonly known as γ-rays, X-rays, and/or the directed delivery of radioisotopes to tumor cells. Other forms of DNA damaging factors are also contemplated such as microwaves and UV-irradiation. It is most likely that all of these factors effect a broad range of damage on DNA, on the precursors of DNA, on the replication and repair of DNA, and on the assembly and maintenance of chromosomes. Dosage ranges for X-rays range from daily doses of 50 to 200 roentgens for prolonged periods of time (3 to 4 wk), to single doses of 2000 to 6000 roentgens. Dosage ranges for radioisotopes vary widely, and depend on the half-life of the isotope, the strength and type of radiation emitted, and the uptake by the neoplastic cells. 101011 The terms “contacted” and “exposed,” when applied to a cell, are used herein to describe the process by which a therapeutic construct and a chemotherapeutic or radiotherapeutic agent are delivered to a target cell or are placed in direct juxtaposition with the target cell. To achieve cell killing or stasis, both agents are delivered to a cell in a combined amount effective to kill the cell or prevent it from dividing.

C. Immunotherapy

Immunotherapeutics, generally, rely on the use of immune effector cells and molecules to target and destroy cancer cells. The immune effector may be, for example, an antibody specific for some marker on the surface of a tumor cell. The antibody alone may serve as an effector of therapy or it may recruit other cells to actually effect cell killing. The antibody also may be conjugated to a drug or toxin (chemotherapeutic, radionuclide, ricin A chain, cholera toxin, pertussis toxin, etc.) and serve merely as a targeting agent. Alternatively, the effector may be a lymphocyte carrying a surface molecule that interacts, either directly or indirectly, with a tumor cell target. Various effector cells include cytotoxic T cells and NK cells.

Immunotherapy, thus, could be used as part of a combined therapy, in conjunction with Ad-p21^{Cip1/WAF1} gene therapy. The general approach for combined therapy is discussed below. Generally, the tumor cell must bear some marker that is amenable to targeting, i.e., is not present on the majority of other cells. Many tumor markers exist and any of these may be suitable for targeting in the context of the present invention. Common tumor markers include carcinoembryonic antigen, prostate specific antigen, urinary tumor associated antigen, fetal antigen, tyrosinase (p97), gp68, TAG-72, HMFG, Sialyl Lewis Antigen, MucA, MucB, PLAP, estrogen receptor, laminin receptor, erb B and p155.

D. Genes

In yet another embodiment, the secondary treatment is a secondary gene therapy in which a second therapeutic polynucleotide is administered before, after, or at the same time a first therapeutic polynucleotide encoding all of part of a mutant form of p21^{Cip1/WAF1}. Delivery of a vector encoding either a full length or truncated mutant form of p21^{Cip1/WAF1} in conjuction with a second vector encoding one of the following gene products will have a combined anti-hyperproliferative effect on target tissues. Alternatively, a single vector encoding both genes may be used. A variety of proteins are encompassed within the invention, some of which are described below.

1. Inducers of Cellular Proliferation

The proteins that induce cellular proliferation further fall into various categories dependent on function. The commonality of all of these proteins is their ability to regulate cellular proliferation. For example, a form of PDGF, the sis oncogene, is a secreted growth factor. Oncogenes rarely arise from genes encoding growth factors, and at the present, sis is the only known naturally-occurring oncogenic growth factor. In one embodiment of the present invention, it is contemplated that anti-sense mRNA directed to a particular inducer of cellular proliferation is used to prevent expression of the inducer of cellular proliferation.

The proteins FMS, ErbA, ErbB and neu are growth factor receptors. Mutations to these receptors result in loss of regulatable function. For example, a point mutation affecting the transmembrane domain of the Neu receptor protein results in the neu oncogene. The erbA oncogene is derived from the intracellular receptor for thyroid hormone. The modified oncogenic ErbA receptor is believed to compete with the endogenous thyroid hormone receptor, causing uncontrolled growth.

The largest class of oncogenes includes the signal transducing proteins (e.g., Src, Abl and Ras). The protein Src is a cytoplasmic protein-tyrosine kinase, and its transformation from proto-oncogene to oncogene in some cases, results via mutations at tyrosine residue 527. In contrast, transformation of GTPase protein ras from proto-oncogene to oncogene, in one example, results from a valine to glycine mutation at amino acid 12 in the sequence, reducing ras GTPase activity.

The proteins Jun, Fos and Myc are proteins that directly exert their effects on nuclear functions as transcription factors.

2. Inhibitors of Cellular Proliferation

The tumor suppressor oncogenes function to inhibit excessive cellular proliferation. The inactivation of these genes destroys their inhibitory activity, resulting in unregulated proliferation. The tumor suppressors p53, p16 and C-CAM are described below.

High levels of mutant p53 have been found in many cells transformed by chemical carcinogenesis, ultraviolet radiation, and several viruses. The p53 gene is a frequent target of mutational inactivation in a wide variety of human tumors and is already documented to be the most frequently mutated gene in common human cancers. It is mutated in over 50% of human NSCLC (Hollstein et al., 1991) and in a wide spectrum of other tumors.

The p53 gene encodes a 393-amino acid phosphoprotein that can form complexes with host proteins such as large-T antigen and E1B. The protein is found in normal tissues and cells, but at concentrations which are minute by comparison with transformed cells or tumor tissue.

Wild-type p53 is recognized as an important growth regulator in many cell types. Missense mutations are common for the p53 gene and are essential for the transforming ability of the oncogene. A single genetic change prompted by point mutations can create carcinogenic p53. Unlike other oncogenes, however, p53 point mutations are known to occur in at least 30 distinct codons, often creating dominant alleles that produce shifts in cell phenotype without a reduction to homozygosity. Additionally, many of these dominant negative alleles appear to be tolerated in the organism and passed on in the germ line. Various mutant alleles appear to range from minimally dysfunctional to strongly penetrant, dominant negative alleles (Weinberg, 1991).

Another inhibitor of cellular proliferation is p16. The major transitions of the eukaryotic cell cycle are triggered by cyclin-dependent kinases, or CDK's. One CDK, cyclin-dependent kinase 4 (CDK4), regulates progression through the G₁. The activity of this enzyme may be to phosphorylate Rb at late G₁. The activity of CDK4 is controlled by an activating subunit, D-type cyclin, and by an inhibitory subunit, the p16^{INK4} has been biochemically characterized as a protein that specifically binds to and inhibits CDK4, and thus may regulate Rb phosphorylation (Serrano et al., 1993; Serrano et al., 1995). Since the p16^{INK4} protein is a CDK4 inhibitor (Serrano, 1993), deletion of this gene may increase the activity of CDK4, resulting in hyperphosphorylation of the Rb protein. p16 also is known to regulate the function of CDK6.

p16^{INK4} belongs to a newly described class of CDK-inhibitory proteins that also includes p¹⁶^{B}, p19, p21^{WAF1}, and p27^{KIP1}. The p16^{INK4} gene maps to 9p21, a chromosome region frequently deleted in many tumor types. Homozygous deletions and mutations of the p16^{INK4} gene are frequent in human tumor cell lines. This evidence suggests that the p16^{INK4} gene is a tumor suppressor gene. This interpretation has been challenged, however, by the observation that the frequency of the p16^{INK4} gene alterations is much lower in primary uncultured tumors than in cultured cell lines (Caldas et al., 1994; Cheng et al., 1994; Hussussian et al., 1994; Kamb et al., 1994; Kamb et al., 1994; Mori et al., 1994; Okamoto et al., 1994; Nobori et al, 1995; Orlow et al., 1994; Arap et al., 1995). Restoration of wild-type p16^{INK4} function by transfection with a plasmid expression vector reduced colony formation by some human cancer cell lines (Okamoto, 1994; Arap, 1995).

Other genes that may be employed according to the present invention include Rb, APC, DCC, NF-1, NF-2, WT-1, MEN-I, MEN-II, zac1, p73, VHL, MMAC1/PTEN, DBCCR-1, FCC, rsk-3, p27, p27/p16 fusions, p21/p27 fusions, anti-thrombotic genes (e.g., COX-1, TFP1), PGS, Dp, E2F, ras, myc, neu, raf, erb, fms, trk, ret, gsp, hst, abl, E1A, p300, genes involved in angiogenesis (e.g., VEGF, FGF, thrombospondin, BAI-1, GDAIF, or their receptors) and MCC.

3. Regulators of Programmed Cell Death

Apoptosis, or programmed cell death, is an essential process for normal embryonic development, maintaining homeostasis in adult tissues, and suppressing carcinogenesis (Kerr et al., 1972). The Bcl-2 family of proteins and ICE-like proteases have been demonstrated to be important regulators and effectors of apoptosis in other systems. The Bcl-2 protein, discovered in association with follicular lymphoma, plays a prominent role in controlling apoptosis and enhancing cell survival in response to diverse apoptotic stimuli (Bakhshi et al., 1985; Cleary and Sklar, 1985; Cleary et al., 1986; Tsujimoto et al., 1985; Tsujimoto and Croce, 1986). The evolutionarily conserved Bcl-2 protein now is recognized to be a member of a family of related proteins, which can be categorized as death agonists or death antagonists.

Subsequent to its discovery, it was shown that Bcl-2 acts to suppress cell death triggered by a variety of stimuli. Also, it now is apparent that there is a family of Bcl-2 cell death regulatory proteins which share in common structural and sequence homologies. These different family members have been shown to either possess similar functions to Bcl-2 (e.g., Bcl_{XL}, Bcl_{W}, Bcl_{S}, Mcl-1, A1, Bfl-1) or counteract Bcl-2 function and promote cell death (e.g., Bax, Bak, Bik, Bim, Bid, Bad, Harakiri). 4. Surgery

Approximately 60% of persons with cancer will undergo surgery of some type, which includes preventative, diagnostic or staging, curative and palliative surgery. Curative surgery is a cancer treatment that may be used in conjunction with other therapies, such as the treatment of the present invention, chemotherapy, radiotherapy, hormonal therapy, gene therapy, immunotherapy and/or alternative therapies.

Curative surgery includes resection in which all or part of cancerous tissue is physically removed, excised, and/or destroyed. Tumor resection refers to physical removal of at least part of a tumor. In addition to tumor resection, treatment by surgery includes laser surgery, cryosurgery, electrosurgery, and miscopically controlled surgery (Mohs' surgery). It is further contemplated that the present invention may be used in conjunction with removal of superficial cancers, precancers, or incidental amounts of normal tissue.

Upon excision of part of all of cancerous cells, tissue, or tumor, a cavity may be formed in the body. Treatment may be accomplished by perfusion, direct injection or local application of the area with an additional anti-cancer therapy. Such treatment may be repeated, for example, every 1, 2, 3, 4, 5, 6, or 7 days, or every 1, 2, 3, 4, and 5 weeks or every 1, 2, 3, 4, 5, 6, 7, 8, 9, 10, 11, or 12 months. These treatments may be of varying dosages as well.

5. Other agents

It is contemplated that other agents may be used in combination with the present invention to improve the therapeutic efficacy of treatment. These additional agents include immunomodulatory agents, agents that affect the upregulation of cell surface receptors and GAP junctions, cytostatic and differentiation agents, inhibitors of cell adehesion, or agents that increase the sensitivity of the hyperproliferative cells to apoptotic inducers. Immunomodulatory agents include tumor necrosis factor; interferon alpha, beta, and gamma; IL-2 and other cytokines; F42K and other cytokine analogs; or MIP-1, MIP-1beta, MCP-1, RANTES, and other chemokines. It is further contemplated that the upregulation of cell surface receptors or their ligands such as Fas/Fas ligand, DR4 or DR5/TRAIL would potentiate the apoptotic inducing abililties of the present invention by establishment of an autocrine or paracrine effect on hyperproliferative cells. Increases intercellular signaling by elevating the number of GAP junctions would increase the anti- hyperproliferative effects on the neighboring hyperproliferative cell population. In other embodiments, cytostatic or differentiation agents can be used in combination with the present invention to improve the anti-hyerproliferative efficacy of the treatments. Inhibitors of cell adehesion are contemplated to improve the efficacy of the present invention. Examples of cell adhesion inhibitors are focal adhesion kinase (FAKs) inhibitors and Lovastatin. It is further contemplated that other agents that increase the sensitivity of a hyperproliferative cell to apoptosis, such as the antibody c225, could be used in combination with the present invention to improve the treatment efficacy.

Hormonal therapy may also be used in conjunction with the present invention or in combination with any other cancer therapy previously described. The use of hormones may be employed in the treatment of certain cancers such as breast, prostate, ovarian, or cervical cancer to lower the level or block the effects of certain hormones such as testosterone or estrogen. This treatment is often used in combination with at least one other cancer therapy as a treatment option or to reduce the risk of metastases.

TABLE 2
Oncogenes

| | | | |
|---|---|---|---|
| Gene | Source | Human Disease | Function |

Growth Factors¹

| | | |
|---|---|---|
| HST/KS | Transfection | FGF family member |
| INT-2 | MMTV promoter | FGF family member |
| | Insertion | |
| INTI/WNTI | MMTV promoter | Factor-like |
| | Insertion | |
| SIS | Simian sarcoma | PDGF B |
| | virus | |

Receptor Tyrosine Kinases^{1, 2}

| | | | |
|---|---|---|---|
| ERBB/HER | Avian | Amplified, deleted | EGF/TGF-α/ |
| | erythroblastosis | squamous cell | amphiregulin/ |
| | virus; ALV | cancer; | hetacellulin |
| | promoter | glioblastoma | receptor |
| | insertion; | | |
| | amplified | | |
| | human tumors | | |
| ERBB-2/NEU/HER-2 | Transfected from rat | Amplified breast, | Regulated by NDF/ |
| | Glioblatoms | ovarian, gastric | heregulin and |
| | | cancers | EGF- |
| | | | related factors |
| FMS | SM feline sarcoma | | CSF-1 receptor |
| | virus | | |
| KIT | HZ feline sarcoma | | MGF/Steel receptor |
| | virus | | hematopoieis |
| TRK | Transfection from | | NGF (nerve growth |
| | human colon | | factor) receptor |
| | cancer | | |
| MET | Transfection from | | Scatter factor/HGF |
| | human | | receptor |
| | osteosarcoma | | |
| RET | Translocations and | Sporadic thyroid | Orphan receptor Tyr |
| | point mutations | cancer; | kinase |
| | | familial medullary | |
| | | thyroid cancer; | |
| | | multiple endocrine | |
| | | neoplasias 2A and | |
| | | 2B | |
| ROS | URII avian sarcoma | | Orphan receptor Tyr |
| | Virus | | kinase |
| PDGF receptor | Translocation | Chronic | TEL(ETS-like |
| | | myclomonocytic | transcription |
| | | leukemia | factor)/ |
| | | | PDGF receptor |
| | | | gene |
| | | | fusion |
| TGF-β receptor | | Colon carcinoma | |
| | | mismatch mutation | |
| | | target | |

NONRECEPTOR TYROSINE KINASES¹

| | | | |
|---|---|---|---|
| ABI. | Abelson Mul. V | Chronic | Interact with RB, |
| | | myelogenous | RNA |
| | | leukemia | polymerase, CRK, |
| | | translocation | CBL |
| | | with BCR | |
| FPS/FES | Avian Fujinami | | |
| | SV; GA | | |
| | FeSV | | |
| LCK | Mul. V (murine | | Src family; T cell |
| | leukemia | | signaling; interacts |
| | virus) promoter | | CD4/CD8 T cells |
| | insertion | | |
| SRC | Avian Rous | | Membrane- |
| | sarcoma | | associated Tyr |
| | Virus | | kinase with |
| | | | signaling function; |
| | | | activated by |
| | | | receptor kinases |
| YES | Avian Y73 virus | | Src family; |
| | | | signaling |

SER/THR PROTEIN KINASES¹

| | | |
|---|---|---|
| AKT | AKT8 murine | Regulated by |
| | retrovirus | PI(3)K?; |
| | | regulate 70-kd S6 |
| | | k? |
| MOS | Maloney murine SV | GVBD; cystostatic |
| | | factor; MAP |
| | | kinase |
| | | kinase |
| PIM-1 | Promoter insertion | |
| | Mouse | |
| RAF/MIL | 3611 murine SV; | Signaling in RAS |
| | MH2 | pathway |
| | avian SV | |

MISCELLANEOUS CELL SURFACE¹

| | | | |
|---|---|---|---|
| APC | Tumor suppressor | Colon cancer | Interacts with |
| | | | catenins |
| DCC | Tumor suppressor | Colon cancer | CAM domains |
| E-cadherin | Candidate tumor | Breast cancer | Extracellular |
| | Suppressor | | homotypic |
| | | | binding; |
| | | | intracellular |
| | | | interacts with |
| | | | catenins |
| PTC/NBCCS | Tumor suppressor | Nevoid basal cell | 12 transmembrane |
| | and | cancer | domain; signals |
| | Drosophilia | syndrome (Gorline | through Gli |
| | homology | syndrome) | homogue |
| | | | CI to antagonize |
| | | | hedgehog pathway |
| TAN-1 Notch | Translocation | T-ALI. | Signaling? |
| homologue | | | |

MISCELLANEOUS SIGNALING^{1, 3}

| | | | |
|---|---|---|---|
| BCL-2 | Translocation | B-cell lymphoma | Apoptosis |
| CBL | Mu Cas NS-1 V | | Tyrosine- |
| | | | phosphorylated |
| | | | RING |
| | | | finger interact Abl |
| CRK | CT1010 ASV | | Adapted SH2/SH3 |
| | | | interact Abl |
| DPC4 | Tumor suppressor | Pancreatic cancer | TGF-β-related |
| | | | signaling |
| | | | pathway |
| MAS | Transfection and | | Possible angiotensin |
| | Tumorigenicity | | receptor |
| NCK | | | Adaptor SH2/SH3 |

GUANINE NUCLEOTIDE EXCHANGERS AND BINDING PROTEINS^{3, 4}

| | | | |
|---|---|---|---|
| BCR | | Translocated with | Exchanger; protein |
| | | ABL in CML | kinase |
| DBL | Transfection | | Exchanger |
| GSP | | | |
| NF-1 | Hereditary tumor | Tumor suppressor | RAS GAP |
| | Suppressor | neurofibromatosis | |
| OST | Transfection | | Exchanger |
| Harvey-Kirsten, N- | HaRat SV; Ki | Point mutations in | Signal cascade |
| RAS | RaSV; | many | |
| | Balb-MoMuSV; | human tumors | |
| | Transfection | | |
| VAV | Transfection | | S112/S113; |
| | | | exchanger |

NUCLEAR PROTEINS AND TRANSCRIPTION FACTORS^{1, 5-9}

| | | | |
|---|---|---|---|
| BRCA1 | Heritable suppressor | Mammary | Localization |
| | | cancer/ovarian | unsettled |
| | | cancer | |
| BRCA2 | Heritable suppressor | Mammary cancer | Function unknown |
| ERBA | Avian | | thyroid hormone |
| | erythroblastosis | | receptor |
| | Virus | | (transcription) |
| ETS | Avian E26 virus | | DNA binding |
| EVII | MuLV promoter | AML | Transcription factor |
| | Insertion | | |
| FOS | FBI/FBR murine | | 1 transcription |
| | osteosarcoma | | factor |
| | viruses | | with c-JUN |
| GLI | Amplified glioma | Glioma | Zinc finger; cubitus |
| | | | interruptus |
| | | | homologue |
| | | | is in hedgehog |
| | | | signaling pathway; |
| | | | inhibitory link |
| | | | PTC |
| | | | and hedgehog |
| HMGG/LIM | Translocation | Lipoma | Gene fusions high |
| | t(3:12) | | mobility group |
| | t(12:15) | | HMGI-C (XT- |
| | | | hook) |
| | | | and transcription |
| | | | factor |
| | | | LIM or acidic |
| | | | domain |
| JUN | ASV-17 | | Transcription factor |
| | | | AP-1 with FOS |
| MLL/VHRX + | Translocation/fusion | Acute myeloid | Gene fusion of |
| ELI/MEN | ELL with MLL | leukemia | DNA- |
| | Trithorax-like gene | | binding and methyl |
| | | | transferase MLL |
| | | | with |
| | | | ELI RNA pol II |
| | | | elongation factor |
| MYB | Avian | | DNA binding |
| | myeloblastosis | | |
| | Virus | | |
| MYC | Avian MC29; | Burkitt's lymphoma | DNA binding with |
| | Translocation B- | | MAX partner; |
| | cell | | cyclin |
| | Lymphomas; | | regulation; interact |
| | promoter | | RB?; regulate |
| | Insertion avian | | apoptosis? |
| | leukosis | | |
| | Virus | | |
| N-MYC | Amplified | Neuroblastoma | |
| L-MYC | | Lung cancer | |
| REL | Avian | | NF-κB family |
| | Retriculoendotheliosis | | transcription factor |
| | Virus | | |
| SKI | Avian SKV770 | | Transcription factor |
| | Retrovirus | | |
| VHL | Heritable suppressor | Von Hippel-Landau | Negative regulator |
| | | syndrome | or elongin; |
| | | | transcriptional |
| | | | elongation |
| | | | complex |
| WT-1 | | Wilm's tumor | Transcription factor |

CELL CYCLE/DNA DAMAGE RESPONSE¹⁰⁻²¹

| | | | |
|---|---|---|---|
| ATM | Hereditary disorder | Ataxia- | Protein/lipid kinase |
| | | telangiectasia | homology; DNA |
| | | | damage response |
| | | | upstream in P53 |
| | | | pathway |
| BCL-2 | Translocation | Follicular | Apoptosis |
| | | lymphoma | |
| FACC | Point mutation | Fanconi's anemia | |
| | | group | |
| | | C (predisposition | |
| | | leukemia | |
| FHIT | Fragile site 3p14.2 | Lung carcinoma | Histidine triad- |
| | | | related |
| | | | diadenosine |
| | | | 5′,3″″- |
| | | | P¹.p⁴ |
| | | | tetraphosphate |
| | | | asymmetric |
| | | | hydrolase |
| hMLI/MutL | | HNPCC | Mismatch repair; |
| | | | MutL |
| | | | homologue |
| hMSH2/MutS | | HNPCC | Mismatch repair; |
| | | | MutS |
| | | | homologue |
| hPMS1 | | HNPCC | Mismatch repair; |
| | | | MutL |
| | | | homologue |
| HPMS2 | | HNPCC | Mismatch repair; |
| | | | MutL |
| | | | homologue |
| INK4/MTS1 | Adjacent INK-4B at | Candidate MTS1 | p16 CDK inhibitor |
| | 9p21; CDK | suppressor and | |
| | complexes | MLM | |
| | | melanoma gene | |
| INK4B/MTS2 | | Candidate | p15 CDK inhibitor |
| | | suppressor | |
| MDM-2 | Amplified | Sarcoma | Negative regulator |
| | | | p53 |
| p53 | Association with | Mutated >50% | Transcription factor; |
| | SV40 | human | checkpoint control; |
| | T antigen | tumors, including | apoptosis |
| | | hereditary Li- | |
| | | Fraumeni | |
| | | syndrome | |
| PRAD1/BCL1 | Translocation with | Parathyroid | Cyclin D |
| | Parathyroid | adenoma; | |
| | hormone or IgG | B-CLL | |
| RB | Hereditary | Retinoblastoma; | Interact cyclin/cdk; |
| | Retinoblastoma; | osteosarcoma; | regulate E2F |
| | Association with | breast | transcription factor |
| | many | cancer; other | |
| | DNA virus tumor | sporadic | |
| | Antigens | cancers | |
| XPA | | xeroderma | Excision repair; |
| | | pigmentosum; skin | photo- |
| | | cancer | product |
| | | predisposition | recognition; |
| | | | zinc finger |

VI. Chemotherapeutic Agents Useful in Neoplastic Disease

A skilled artisan recognizes that in addition to the p21^{Cip1/WAF1} mutant forms described herein for the purpose of inhibiting cell growth, other chemotherapeutic agents are useful in the treatment of neoplastic disease. Examples of such chemotherapeutic agents are described in the following table.

TABLE 3
Chemotherapeutic Agents Useful In Neoplastic Disease

| | | NONPROPRIETARY | |
|---|---|---|---|
| | TYPE OF | NAMES | |
| CLASS | AGENT | (OTHER NAMES) | DISEASE |
| Alkylating | Nitrogen | Mechlorethamine | Hodgkin's disease, |
| Agents | Mustards | (HN₂) | non-Hodgkin's lymphomas |
| | | Cyclophosphamide | Acute and chronic |
| | | Ifosfamide | lymphocytic leukemias, |
| | | | Hodgkin's disease, |
| | | | non-Hodgkin's |
| | | | lymphomas, multiple |
| | | | myeloma, neuroblastoma, |
| | | | breast, ovary, lung, Wilms' |
| | | | tumor, cervix, testis, |
| | | | soft-tissue sarcomas |
| | | Melphalan | Multiple myeloma, breast, |
| | | (L-sarcolysin) | ovary |
| | | Chlorambucil | Chronic lymphocytic |
| | | | leukemia, primary |
| | | | macroglobulinemia, |
| | | | Hodgkin's disease, |
| | | | non-Hodgkin's lymphomas |
| | Ethylenimenes | Hexamethylmelamine | Ovary |
| | and | Thiotepa | Bladder, breast, ovary |
| | Methylmela- | | |
| | mines | | |
| | Alkyl Sulfonates | Busulfan | Chronic granulocytic |
| | | | leukemia |
| | Nitrosoureas | Carmustine (BCNU) | Hodgkin's disease, |
| | | | non-Hodgkin's |
| | | | lymphomas, primary brain |
| | | | tumors, multiple myeloma, |
| | | | malignant melanoma |
| | | Lomustine (CCNU) | Hodgkin's disease, |
| | | | non-Hodgkin's |
| | | | lymphomas, primary brain |
| | | | tumors, small-cell lung |
| | | Semustine | Primary brain tumors, |
| | | (methyl-CCNU) | stomach, colon |
| | | Streptozocin | Malignant pancreatic |
| | | (streptozotocin) | insulinoma, malignant |
| | | | carcinoid |
| | Triazines | Dacarbazine (DTIC; | Malignant melanoma, |
| | | dimethyltriazenoimida | Hodgkin's disease, |
| | | zolecarboxamide) | soft-tissue sarcomas |
| Antimetabolites | Folic Acid | Methotrexate | Acute lymphocytic |
| | Analogs | (amethopterin) | leukemia, choriocarcinoma, |
| | | | mycosis fungoides, breast, |
| | | | head and neck, lung, |
| | | | osteogenic sarcoma |
| | Pyrimidine | Fluouracil | Breast, colon, stomach, |
| | Analogs | (5-fluorouracil; 5-FU) | pancreas, ovary, head and |
| | | Floxuridine | neck, urinary bladder, |
| | | (fluorode-oxyuridine; | premalignant skin lesions |
| | | FUdR) | (topical) |
| | | Cytarabine (cytosine | Acute granulocytic and |
| | | arabinoside) | acute lymphocytic |
| | | | leukemias |
| | Purine Analogs | Mercaptopurine | Acute lymphocytic, acute |
| | and Related | (6-mercaptopurine; | granulocytic and chronic |
| | Inhibitors | 6-MP) | granulocytic leukemias |
| | | Thioguanine | Acute granulocytic, acute |
| | | (6-thioguanine; TG) | lymphocytic and chronic |
| | | | granulocytic leukemias |
| | | Pentostatin | Hairy cell leukemia, |
| | | (2-deoxycoformycin) | mycosis fungoides, chronic |
| | | | lymphocytic leukemia |
| Natural | Vinca Alkaloids | Vinblastine (VLB) | Hodgkin's disease, |
| Products | | | non-Hodgkin's |
| | | | lymphomas, breast, testis |
| | | Vincristine | Acute lymphocytic |
| | | | leukemia, neuroblastoma, |
| | | | Wilms' tumor, |
| | | | rhabdomyosarcoma, |
| | | | Hodgkin's disease, |
| | | | non-Hodgkin's |
| | | | lymphomas, small-cell lung |
| | Epipodophyllo- | Etoposide | Testis, small-cell lung and |
| | toxins | Tertiposide | other lung, breast, |
| | | | Hodgkin's disease, |
| | | | non-Hodgkin's |
| | | | lymphomas, acute |
| | | | granulocytic leukemia, |
| | | | Kaposi's sarcoma |
| | Antibiotics | Dactinomycin | Choriocarcinoma, Wilms' |
| | | (actinomycin D) | tumor, rhabdomyosarcoma, |
| | | | testis, Kaposi's sarcoma |
| | | Daunorubicin | Acute granulocytic and |
| | | (daunomycin; | acute lymphocytic |
| | | rubidomycin) | leukemias |
| | | Doxorubicin | Soft-tissue, osteogenic and |
| | | | other sarcomas; Hodgkin's |
| | | | disease, non-Hodgkin's |
| | | | lymphomas, acute |
| | | | leukemias, breast, |
| | | | genitourinary, thyroid, |
| | | | lung, stomach, |
| | | | neuroblastoma |
| | | Bleomycin | Testis, head and neck, skin, |
| | | | esophagus, lung and |
| | | | genitourinary tract; |
| | | | Hodgkin's disease, |
| | | | non-Hodgkin's lymphomas |
| | | Plicamycin | Testis, malignant |
| | | (mithramycin) | hypercalcemia |
| | | Mitomycin | Stomach, cervix, colon, |
| | | (mitomycin C) | breast, pancreas, bladder, |
| | | | head and neck |
| | Enzymes | L-Asparaginase | Acute lymphocytic |
| | | | leukemia |
| | Biological | Interferon alfa | Hairy cell leukemia., |
| | Response | | Kaposi's sarcoma, |
| | Modifiers | | melanoma, carcinoid, renal |
| | | | cell, ovary, bladder, |
| | | | non-Hodgkin's |
| | | | lymphomas, mycosis |
| | | | fungoides, multiple |
| | | | myeloma, chronic |
| | | | granulocytic leukemia |
| Miscellaneous | Platinum | Cisplatin (cis-DDP) | Testis, ovary, bladder, head |
| Agents | Coordination | Carboplatin | and neck, lung, thyroid, |
| | Complexes | | cervix, endometrium, |
| | | | neuroblastoma, osteogenic |
| | | | sarcoma |
| | Anthracene- | Mitoxantrone | Acute granulocytic |
| | dione | | leukemia, breast |
| | Substituted Urea | Hydroxyurea | Chronic granulocytic |
| | | | leukemia, polycythemia |
| | | | vera, essental |
| | | | thrombocytosis, malignant |
| | | | melanoma |
| | Methyl | Procarbazine | Hodgkin's disease |
| | Hydrazine | (N-methylhydrazine, | |
| | Derivative | MIH) | |
| | Adrenocortical | Mitotane (o,p′-DDD) | Adrenal cortex |
| | Suppressant | Aminoglutethimide | Breast |
| Hormones and | Adrenocorti | Prednisone (several | Acute and chronic |
| Antagonists | costeroids | other equivalent | lymphocytic leukemias, |
| | | preparations available) | non-Hodgkin's |
| | | | lymphomas, Hodgkin's |
| | | | disease, breast |
| | Progestins | Hydroxyprogesterone | Endometrium, breast |
| | | caproate | |
| | | Medroxyprogesterone | |
| | | acetate | |
| | | Megestrol acetate | |
| | Estrogens | Diethylstilbestrol | Breast, prostate |
| | | Ethinyl estradiol (other | |
| | | preparations available) | |
| | Antiestrogen | Tamoxifen | Breast |
| | Androgens | Testosterone | Breast |
| | | propionate | |
| | | Fluoxymesterone | |
| | | (other preparations | |
| | | available) | |
| | Antiandrogen | Flutamide | Prostate |
| | Gonadotropin- | Leuprolide | Prostate |
| | releasing | | |
| | hormone analog | | |

VII. Cancer Therapies

A wide variety of cancer therapies, known to one of skill in the art, may be used in combination with the p21 mutant forms contemplated for use in the present invention. The inventors can use any of the treatments described below on an individual stricken with a proliferative cellular disorder while evaluating the efficacy of p21 mutant forms, before, during and/or after treatment with a p21 mutant form.

A. Radiotherapeutic agents

Radiotherapeutic agents and factors include radiation and waves that induce DNA damage for example, γ-irradiation, X-rays, UV-irradiation, microwaves, electronic emissions, radioisotopes, and the like. Therapy may be achieved by irradiating the localized tumor site with the above described forms of radiations. It is most likely that all of these factors effect a broad range of damage DNA, on the precursors of DNA, the replication and repair of DNA, and the assembly and maintenance of chromosomes.

Dosage ranges for X-rays range from daily doses of 50 to 200 roentgens for prolonged periods of time (3 to 4 weeks), to single doses of 2000 to 6000 roentgens. Dosage ranges for radioisotopes vary widely, and depend on the half-life of the isotope, the strength and type of radiation emitted, and the uptake by the neoplastic cells.

B. Surgery

Surgical treatment for removal of the cancerous growth is generally a standard procedure for the treatment of tumors and cancers. This attempts to remove the entire cancerous growth. However, surgery is generally combined with chemotherapy and/or radiotherapy to ensure the destruction of any remaining neoplastic or malignant cells. Thus, surgery or sham surgery may be used in the model in the context of the present invention.

C. Chemotherapeutic Agents

These can be, for example, agents that directly cross-link DNA, agents that intercalate into DNA, and agents that lead to chromosomal and mitotic aberrations by affecting nucleic acid synthesis.

Agents that directly cross-link nucleic acids, specifically DNA, are envisaged and are shown herein, to eventuate DNA damage leading to a synergistic antineoplastic combination. Agents such as cisplatin, and other DNA alkylating agents may be used.

Agents that damage DNA also include compounds that interfere with DNA replication, mitosis, and chromosomal segregation. Examples of these compounds include adriamycin (also known as doxorubicin), VP-16 (also known as etoposide), verapamil, podophyllotoxin, and the like. Widely used in clinical setting for the treatment of neoplasms these compounds are administered through bolus injections intravenously at doses ranging from 25-75 mg/m² at 21 day intervals for adriamycin, to 35-100 mg/m² for etoposide intravenously or orally.

D. Antibiotics

1. Doxorubicin

Doxorubicin hydrochloride, 5,12-Naphthacenedione, (8s-cis)-10-[(3-amino-2,3,6-trideoxy-a-L-lyxo-hexopyranosyl)oxy]-7,8,9,1 0-tetrahydro-6,8,11-trihydroxy-8-(hydroxyacetyl)-1-methoxy-hydrochloride (hydroxydaunorubicin hydrochloride, Adriamycin) is used in a wide antineoplastic spectrum. It binds to DNA and inhibits nucleic acid synthesis, inhibits mitosis and promotes chromosomal aberrations.

Administered alone, it is the drug of first choice for the treatment of thyroid adenoma and primary hepatocellular carcinoma. It is a component of 31 first-choice combinations for the treatment of ovarian, endometrial and breast tumors, bronchogenic oat- cell carcinoma, non-small cell lung carcinoma, gastric adenocarcinoma, retinoblastoma, neuroblastoma, mycosis fungoides, pancreatic carcinoma, prostatic carcinoma, bladder carcinoma, myeloma, diffuse histiocytic lymphoma, Wilms' tumor, Hodgkin's disease, adrenal tumors, osteogenic sarcoma soft tissue sarcoma, Ewing's sarcoma, rhabdomyosarcoma and acute lymphocytic leukemia. It is an alternative drug for the treatment of islet cell, cervical, testicular and adrenocortical cancers. It is also an immunosuppressant.

Doxorubicin is absorbed poorly and must be administered intravenously. The pharmacokinetics are multicompartmental. Distribution phases have half-lives of 12 minutes and 3.3 hr. The elimination half-life is about 30 hr. Forty to 50% is secreted into the bile. Most of the remainder is metabolized in the liver, partly to an active metabolite (doxorubicinol), but a few percent is excreted into the urine. In the presence of liver impairment, the dose should be reduced.

Appropriate doses are, intravenous, adult, 60 to 75 mg/m² at 21-day intervals or 25 to 30 mg/m² on each of 2 or 3 successive days repeated at 3- or 4-wk intervals or 20 mg/m² once a week. The lowest dose should be used in elderly patients, when there is prior bone-marrow depression caused by prior chemotherapy or neoplastic marrow invasion, or when the drug is combined with other myelopoietic suppressant drugs. The dose should be reduced by 50% if the serum bilirubin lies between 1.2 and 3 mg/dL and by 75% if above 3 mg/dL. The lifetime total dose should not exceed 550 mg/m² in patients with normal heart function and 400 mg/m² in persons having received mediastinal irradiation. Alternatively, 30 mg/m² on each of 3 consecutive days, repeated every 4 wk. Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m^{2, 475} mg/m², 500 mg/m². Of course, all of these dosages are exemplary, and any dosage in-between these points is also expected to be of use in the invention.

2. Daunorubicin

Daunorubicin hydrochloride, 5,12-Naphthacenedione, (8S-cis)-8-acetyl-10-[(3-amino-2,3,6-trideoxy-a-L-lyxo-hexanopyranosyl)oxy]-7,8,9,10-tetrahydro-6,8,11-trihydroxy-10-methoxy-, hydrochloride; also termed cerubidine and available from Wyeth. Daunorubicin intercalates into DNA, blocks DNA-directed RNA polymerase and inhibits DNA synthesis. It can prevent cell division in doses that do not interfere with nucleic acid synthesis.

In combination with other drugs it is included in the first-choice chemotherapy of acute myelocytic leukemia in adults (for induction of remission), acute lymphocytic leukemia and the acute phase of chronic myclocytic leukemia. Oral absorption is poor, and it must be given intravenously. The half-life of distribution is 45 minutes and of elimination, about 19 hr. The half-life of its active metabolite, daunorubicinol, is about 27 hr. Daunorubicin is metabolized mostly in the liver and also secreted into the bile (ca 40%). Dosage must be reduced in liver or renal insufficiencies.

Suitable doses are (base equivalent), intravenous adult, younger than 60 yr. 45 mg/m²/day (30 mg/m² for patients older than 60 yr.) for 1, 2 or 3 days every 3 or 4 wk or 0.8 mg/kg/day for 3 to 6 days every 3 or 4 wk; no more than 550 mg/m² should be given in a lifetime, except only 450 mg/m² if there has been chest irradiation; children, 25 mg/m² once a week unless the age is less than 2 yr. or the body surface less than 0.5 m, in which case the weight-based adult schedule is used. It is available in injectable dosage forms (base equivalent) 20 mg (as the base equivalent to 21.4 mg of the hydrochloride). Exemplary doses may be 10 mg/m², 20 mg/m², 30 mg/m², 50 mg/m², 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

3. Mitomycin

Mitomycin (also known as mutamycin and/or mitomycin-C) is an antibiotic isolated from the broth of Streptomyces caespitosus which has been shown to have antitumor activity. The compound is heat stable, has a high melting point, and is freely soluble in organic solvents.

Mitomycin selectively inhibits the synthesis of deoxyribonucleic acid (DNA). The guanine and cytosine content correlates with the degree of mitomycin-induced cross-linking. At high concentrations of the drug, cellular RNA and protein synthesis are also suppressed.

In humans, mitomycin is rapidly cleared from the serum after intravenous administration. Time required to reduce the serum concentration by 50% after a 30 mg. bolus injection is 17 minutes. After injection of 30 mg., 20 mg., or 10 mg. I.V., the maximal serum concentrations were 2.4 mg./mL, 1.7 mg./mL, and 0.52 mg./mL, respectively. Clearance is effected primarily by metabolism in the liver, but metabolism occurs in other tissues as well. The rate of clearance is inversely proportional to the maximal serum concentration because, it is thought, of saturation of the degradative pathways.

Approximately 10% of a dose of mitomycin is excreted unchanged in the urine. Since metabolic pathways are saturated at relatively low doses, the percent of a dose excreted in urine increases with increasing dose. In children, excretion of intravenously administered mitomycin is similar. 4. Actinomycin D

Actinomycin D (Dactinomycin) [50-76-0]; C₆₂H₈₆N₁₂O₁₆ (1255.43) is an antineoplastic drug that inhibits DNA-dependent RNA polymerase. It is a component of first-choice combinations for treatment of choriocarcinoma, embryonal rhabdomyosarcoma, testicular tumor and Wilms' tumor. Tumors which fail to respond to systemic treatment sometimes respond to local perfusion. Dactinomycin potentiates radiotherapy. It is a secondary (efferent) immunosuppressive.

Actinomycin D is used in combination with primary surgery, radiotherapy, and other drugs, particularly vincristine and cyclophosphamide. Antineoplastic activity has also been noted in Ewing's tumor, Kaposi's sarcoma, and soft-tissue sarcomas. Dactinomycin can be effective in women with advanced cases of choriocarcinoma. It also produces consistent responses in combination with chlorambucil and methotrexate in patients with metastatic testicular carcinomas. A response may sometimes be observed in patients with Hodgkin's disease and non-Hodgkin's lymphomas. Dactinomycin has also been used to inhibit immunological responses, particularly the rejection of renal transplants.

Half of the dose is excreted intact into the bile and 10% into the urine; the half-life is about 36 hr. The drug does not pass the blood-brain barrier. Actinomycin D is supplied as a lyophilized powder (0/5 mg in each vial). The usual daily dose is 10 to 15 mg/kg; this is given intravenously for 5 days; if no manifestations of toxicity are encountered, additional courses may be given at intervals of 3 to 4 weeks. Daily injections of 100 to 400 mg have been given to children for 10 to 14 days; in other regimens, 3 to 6 mg/kg, for a total of 125 mg/kg, and weekly maintenance doses of 7.5 mg/kg have been used. Although it is safer to administer the drug into the tubing of an intravenous infusion, direct intravenous injections have been given, with the precaution of discarding the needle used to withdraw the drug from the vial in order to avoid subcutaneous reaction. Exemplary doses may be 100 mg/m², 150 mg/m², 175 mg/m², 200 mg/m², 225 mg/m², 250 mg/m², 275 mg/m², 300 mg/m², 350 mg/m², 400 mg/m², 425 mg/m², 450 mg/m², 475 mg/m², 500 mg/m². Of course, all of these dosages are exemplary, and any dosage in-between these points is also expected to be of use in the invention.

5. Bleomycin

Bleomycin is a mixture of cytotoxic glycopeptide antibiotics isolated from a strain of Streptomyces verticillus. It is freely soluble in water.

Although the exact mechanism of action of bleomycin is unknown, available evidence would seem to indicate that the main mode of action is the inhibition of DNA synthesis with some evidence of lesser inhibition of RNA and protein synthesis.

In mice, high concentrations of bleomycin are found in the skin, lungs, kidneys, peritoneum, and lymphatics. Tumor cells of the skin and lungs have been found to have high concentrations of bleomycin in contrast to the low concentrations found in hematopoietic tissue. The low concentrations of bleomycin found in bone marrow may be related to high levels of bleomycin degradative enzymes found in that tissue.

In patients with a creatinine clearance of >35 mL per minute, the serum or plasma terminal elimination half-life of bleomycin is approximately 115 minutes. In patients with a creatinine clearance of <35 mL per minute, the plasma or serum terminal elimination half-life increases exponentially as the creatinine clearance decreases. In humans, 60% to 70% of an administered dose is recovered in the urine as active bleomycin.

Bleomycin should be considered a palliative treatment. It has been shown to be useful in the management of the following neoplasms either as a single agent or in proven combinations with other approved chemotherapeutic agents in squamous cell carcinoma such as head and neck (including mouth, tongue, tonsil, nasopharynx, oropharynx, sinus, palate, lip, buccal mucosa, gingiva, epiglottis, larynx), skin, penis, cervix, and vulva. It has also been used in the treatment of lymphomas and testicular carcinoma.

Because of the possibility of an anaphylactoid reaction, lymphoma patients should be treated with two units or less for the first two doses. If no acute reaction occurs, then the regular dosage schedule may be followed.

Improvement of Hodgkin's Disease and testicular tumors is prompt and noted within 2 weeks. If no improvement is seen by this time, improvement is unlikely. Squamous cell cancers respond more slowly, sometimes requiring as long as 3 weeks before any improvement is noted.

Bleomycin may be given by the intramuscular, intravenous, or subcutaneous routes.

E. Miscellaneous Agents

1. Cisplatin

Cisplatin has been widely used to treat cancers such as metastatic testicular or ovarian carcinoma, advanced bladder cancer, head or neck cancer, cervical cancer, lung cancer or other tumors. Cisplatin can be used alone or in combination with other agents, with efficacious doses used in clinical applications of 15-20 mg/m² for 5 days every three weeks for a total of three courses. Exemplary doses may be 0.50 mg/m², 1.0 mg/m², 1.50 mg/m², 1.75 mg/m², 2.0 mg/m², 3.0 mg/m², 4.0 mg/m², 5.0 mg/m², 10 mg/m². Of course, all of these dosages are exemplary, and any dosage in-between these points is also expected to be of use in the invention.

Cisplatin is not absorbed orally and must therefore be delivered via injection intravenously, subcutaneously, intratumorally or intraperitoneally.

In certain aspects of the current invention cisplatin is used in combination with emodin or emodin-like compounds in the treatment of non-small cell lung carcinoma. It is clear, however, that the combination of cisplatin and emodin and or emodin-like compounds could be used for the treatment of any other neu-mediated cancer.

2. VP16

VP16 is also know as etoposide and is used primarily for treatment of testicular tumors, in combination with bleomycin and cisplatin, and in combination with cisplatin for small-cell carcinoma of the lung. It is also active against non-Hodgkin's lymphomas, acute nonlymphocytic leukemia, carcinoma of the breast, and Kaposi's sarcoma associated with acquired immunodeficiency syndrome (AIDS).

VP16 is available as a solution (20 mg/ml) for intravenous administration and as 50-mg, liquid-filled capsules for oral use. For small-cell carcinoma of the lung, the intravenous dose (in combination therapy) is can be as much as 100 mg/m² or as little as 2 mg/ m², routinely 35 mg/m², daily for 4 days, to 50 mg/m², daily for 5 days have also been used. When given orally, the dose should be doubled. Hence the doses for small cell lung carcinoma may be as high as 200-250 mg/m . The intravenous dose for testicular cancer (in combination therapy) is 50 to 100 mg/m² daily for 5 days, or 100 mg/m² on alternate days, for three doses. Cycles of therapy are usually repeated every 3 to 4 weeks. The drug should be administered slowly during a 30- to 60-minute infusion in order to avoid hypotension and bronchospasm, which are probably due to the solvents used in the formulation.

3. Tumor Necrosis Factor

Tumor Necrosis Factor [TNF; Cachectin] is a glycoprotein that kills some kinds of cancer cells, activates cytokine production, activates macrophages and endothelial cells, promotes the production of collagen and collagenases, is an inflammatory mediator and also a mediator of septic shock, and promotes catabolism, fever and sleep. Some infectious agents cause tumor regression through the stimulation of TNF production. TNF can be quite toxic when used alone in effective doses, so that the optimal regimens probably will use it in lower doses in combination with other drugs. Its immunosuppressive actions are potentiated by gamma-interferon, so that the combination potentially is dangerous. A hybrid of TNF and interferon-α also has been found to possess anti-cancer activity.

F. Plant Alkaloids 1. Taxol

Taxol is an experimental antimitotic agent, isolated from the bark of the ash tree, Taxus brevifolia. It binds to tubulin (at a site distinct from that used by the vinca alkaloids) and promotes the assembly of microtubules. Taxol is currently being evaluated clinically; it has activity against malignant melanoma and carcinoma of the ovary. Maximal doses are 30 mg/m² per day for 5 days or 210 to 250 mg/m² given once every 3 weeks. Of course, all of these dosages are exemplary, and any dosage in-between these points is also expected to be of use in the invention.

2. Vincristine

Vincristine blocks mitosis and produces metaphase arrest. It seems likely that most of the biological activities of this drug can be explained by its ability to bind specifically to tubulin and to block the ability of protein to polymerize into microtubules. Through disruption of the microtubules of the mitotic apparatus, cell division is arrested in metaphase. The inability to segregate chromosomes correctly during mitosis presumably leads to cell death.

The relatively low toxicity of vincristine for normal marrow cells and epithelial cells make this agent unusual among anti-neoplastic drugs, and it is often included in combination with other myelosuppressive agents.

Unpredictable absorption has been reported after oral administration of vinblastine or vincristine. At the usual clinical doses the peak concentration of each drug in plasma is approximately 0.4 mM.

Vinblastine and vincristine bind to plasma proteins. They are extensively concentrated in platelets and to a lesser extent in leukocytes and erythrocytes.

Vincristine has a multiphasic pattern of clearance from the plasma; the terminal half-life is about 24 hours. The drug is metabolized in the liver, but no biologically active derivatives have been identified. Doses should be reduced in patients with hepatic dysfunction. At least a 50% reduction in dosage is indicated if the concentration of bilirubin in plasma is greater than 3 mg/dl (about 50 mM).

Vincristine sulfate is available as a solution (1 mg/ml) for intravenous injection. Vincristine used together with corticosteroids is presently the treatment of choice to induce remissions in childhood leukemia; the optimal dosages for these drugs appear to be vincristine, intravenously, 2 mg/m² of body-surface area, weekly, and prednisolone, orally, 40 mg/m², daily. Adult patients with Hodgkin's disease or non-Hodgkin's lymphomas usually receive vincristine as a part of a complex protocol. When used in the MOPP regimen, the recommended dose of vincristine is 1.4 mg/m². High doses of vincristine seem to be tolerated better by children with leukemia than by adults, who may experience sever neurological toxicity. Administration of the drug more frequently than every 7 days or at higher doses seems to increase the toxic manifestations without proportional improvement in the response rate. Precautions should also be used to avoid extravasation during intravenous administration of vincristine. Vincristine (and vinblastine) can be infused into the arterial blood supply of tumors in doses several times larger than those that can be administered intravenously with comparable toxicity.

Vincristine has been effective in Hodgkin's disease and other lymphomas. Although it appears to be somewhat less beneficial than vinblastine when used alone in Hodgkin's disease, when used with mechlorethamine, prednisolone, and procarbazine (the so-called MOPP regimen), it is the preferred treatment for the advanced stages (III and IV) of this disease. In non-Hodgkin's lymphomas, vincristine is an important agent, particularly when used with cyclophosphamide, bleomycin, doxorubicin, and prednisolone. Vincristine is more useful than vinblastine in lymphocytic leukemia. Beneficial response have been reported in patients with a variety of other neoplasms, particularly Wilms' tumor, neuroblastoma, brain tumors, rhabdomyosarcoma, and carcinomas of the breast, bladder, and the male and female reproductive systems.

Doses of vincristine for use will be determined by the clinician according to the individual patients need. 0.01 to 0.03 mg/kg or 0.4 to 1.4 mg/m² can be administered or 1.5 to 2 mg/m² can alos be administered. Alternatively 0.02 mg/m², 0.05 mg/m², 0.06 mg/m², 0.07 mg/m², 0.08 mg/m², 0.1 mg/m², 0.12 mg/m², 0.14 mg/m², 0.15 mg/m², 0.2 mg/m², 0.25 mg/m² can be given as a constant intravenous infusion. Of course, all of these dosages are exemplary, and any dosage in-between these points is also expected to be of use in the invention.

3. Vinblastine

When cells are incubated with vinblastine, dissolution of the microtubules occurs. Unpredictable absorption has been reported after oral administration of vinblastine or vincristine. At the usual clinical doses the peak concentration of each drug in plasma is approximately 0.4 mM. Vinblastine and vincristine bind to plasma proteins. They are extensively concentrated in platelets and to a lesser extent in leukocytes and erythrocytes.

After intravenous injection, vinblastine has a multiphasic pattern of clearance from the plasma; after distribution, drug disappears from plasma with half-lives of approximately 1 and 20 hours.

Vinblastine is metabolized in the liver to biologically activate derivative desacetylvinblastine. Approximately 15% of an administered dose is detected intact in the urine, and about 10% is recovered in the feces after biliary excretion. Doses should be reduced in patients with hepatic dysfunction. At least a 50% reduction in dosage is indicated if the concentration of bilirubin in plasma is greater than 3 mg/dl (about 50 mM).

Vinblastine sulfate is available in preparations for injection. The drug is given intravenously; special precautions must be taken against subcutaneous extravasation, since this may cause painful irritation and ulceration. The drug should not be injected into an extremity with impaired circulation. After a single dose of 0.3 mg/kg of body weight, myelosuppression reaches its maximum in 7 to 10 days. If a moderate level of leukopenia (approximately 3000 cells/mm³) is not attained, the weekly dose may be increased gradually by increments of 0.05 mg/kg of body weight. In regimens designed to cure testicular cancer, vinblastine is used in doses of 0.3 mg/kg every 3 weeks irrespective of blood cell counts or toxicity.

The most important clinical use of vinblastine is with bleomycin and cisplatin in the curative therapy of metastatic testicular tumors. Beneficial responses have been reported in various lymphomas, particularly Hodgkin's disease, where significant improvement may be noted in 50 to 90% of cases. The effectiveness of vinblastine in a high proportion of lymphomas is not diminished when the disease is refractory to alkylating agents. It is also active in Kaposi's sarcoma, neuroblastoma, and Letterer-Siwe disease (histiocytosis X), as well as in carcinoma of the breast and choriocarcinoma in women.

Doses of vinblastine for use will be determined by the clinician according to the individual patients need. 0.1 to 0.3 mg/kg can be administered or 1.5 to 2 mg/m² can also be administered. Alternatively, 0.1 mg/m², 0.12 mg/m², 0.14 mg/m², 0.15 mg/m², 0.2 mg/m², 0.25 mg/m², 0.5 mg/m², 1.0 mg/m², 1.2 mg/m², 1.4 mg/m², 1.5 mg/m², 2.0 mg/m², 2.5 mg/m², 5.0 Mg/m², 6 Mg/m², 8 mg/m², 9 mg/m², 10 mg/m², 20 mg/m², can be given. Of course, all of these dosages are exemplary, and any dosage in-between these points is also expected to be of use in the invention.

G. Alkylating Agents

1. Carmustine

Carmustine (sterile carmustine) is one of the nitrosoureas used in the treatment of certain neoplastic diseases. It is 1,3bis (2-chloroethyl)-1-nitrosourea. It is lyophilized pale yellow flakes or congealed mass with a molecular weight of 214.06. It is highly soluble in alcohol and lipids, and poorly soluble in water. Carmustine is administered by intravenous infusion after reconstitution as recommended. Sterile carmustine is commonly available in 100 mg single dose vials of lyophilized material.

Although it is generally agreed that carmustine alkylates DNA and RNA, it is not cross resistant with other alkylators. As with other nitrosoureas, it may also inhibit several key enzymatic processes by carbamoylation of amino acids in proteins.

Carmustine is indicated as palliative therapy as a single agent or in established combination therapy with other approved chemotherapeutic agents in brain tumors such as glioblastoma, brainstem glioma, medullobladyoma, astrocytoma, ependymoma, and metastatic brain tumors. Also it has been used in combination with prednisolone to treat multiple myeloma. Carmustine has proved useful, in the treatment of Hodgkin's Disease and in non-Hodgkin's lymphomas, as secondary therapy in combination with other approved drugs in patients who relapse while being treated with primary therapy, or who fail to respond to primary therapy.

The recommended dose of carmustine as a single agent in previously untreated patients is 150 to 200 mg/m² intravenously every 6 weeks. This may be given as a single dose or divided into daily injections such as 75 to 100 mg/m² on 2 successive days. When carmustine is used in combination with other myelosuppressive drugs or in patients in whom bone marrow reserve is depleted, the doses should be adjusted accordingly. Doses subsequent to the initial dose should be adjusted according to the hematologic response of the patient to the preceding dose. It is of course understood that other doses may be used in the present invention for example 10 mg/m², 20 mg/m², 30 mg/m² 40 mg/m² 50 mg/m² 60 mg/m²70 mg/m² 80 mg/m² 90 mg/m^{2 100} mg/m². The skilled artisan is directed to, “Remington's Pharmaceutical Sciences” 15th Edition, chapter 61. Some variation in dosage will necessarily occur depending on the condition of the subject being treated. The person responsible for administration will, in any event, determine the appropriate dose for the individual subject

2. Melphalan

Melphalan also known as alkeran, L-phenylalanine mustard, phenylalanine mustard, L-PAM, or L-sarcolysin, is a phenylalanine derivative of nitrogen mustard. Melphalan is a bifunctional alkylating agent which is active against selective human neoplastic diseases. It is known chemically as 4-[bis(2-chloroethyl)amino]-L-phenylalanine.

Melphalan is the active L-isomer of the compound and was first synthesized in 1953 by Bergel and Stock; the D-isomer, known as medphalan, is less active against certain animal tumors, and the dose needed to produce effects on chromosomes is larger than that required with the L-isomer. The racemic (DL−) form is known as merphalan or sarcolysin. Melphalan is insoluble in water and has a pKa₁ of 2.1. Melphalan is available in tablet form for oral administration and has been used to treat multiple myeloma.

Available evidence suggests that about one third to one half of the patients with multiple myeloma show a favorable response to oral administration of the drug.

Melphalan has been used in the treatment of epithelial ovarian carcinoma. One commonly employed regimen for the treatment of ovarian carcinoma has been to administer melphalan at a dose of 0.2 mg/kg daily for five days as a single course. Courses are repeated every four to five weeks depending upon hematologic tolerance (Smith and Rutledge, 1975; Young et al., 1978). Alternatively the dose of melphalan used could be as low as 0.05 mg/kg/day or as high as 3 mg/kg/day or any dose in between these doses or above these doses. Some variation in dosage will necessarily occur depending on the condition of the subject being treated. The person responsible for administration will, in any event, determine the appropriate dose for the individual subject

3. Cyclophosphamide

Cyclophosphamide is 2H-1,3,2-Oxazaphosphorin-2-amine, N,N-bis(2-chloroethyl)tetrahydro-, 2-oxide, monohydrate; termed Cytoxan available from Mead Johnson; and Neosar available from Adria. Cyclophosphamide is prepared by condensing 3-amino-1-propanol with N,N-bis(2-chlorethyl) phosphoramidic dichloride [(ClCH₂CH₂)₂N—POCl₂] in dioxane solution under the catalytic influence of triethylamine. The condensation is double, involving both the hydroxyl and the amino groups, thus effecting the cyclization.

Unlike other β-chloroethylamino alkylators, it does not cyclize readily to the active ethyleneimonium form until activated by hepatic enzymes. Thus, the substance is stable in the gastrointestinal tract, tolerated well and effective by the oral and parental routes and does not cause local vesication, necrosis, phlebitis or even pain.

Suitable doses for adults include, orally, 1 to 5 mg/kg/day (usually in combination), depending upon gastrointestinal tolerance; or 1 to 2 mg/kg/day; intravenously, initially 40 to 50 mg/kg in divided doses over a period of 2 to 5 days or 10 to 15 mg/kg every 7 to 10 days or 3 to 5 mg/kg twice a week or 1.5 to 3 mg/kg/day. A dose 250 mg/kg/day may be administered as an antineoplastic. Because of gastrointestinal adverse effects, the intravenous route is preferred for loading. During maintenance, a leukocyte count of 3000 to 4000/mm³ usually is desired. The drug also sometimes is administered intramuscularly, by infiltration or into body cavities. It is available in dosage forms for injection of 100, 200 and 500 mg, and tablets of 25 and 50 mg the skilled artisan is referred to “Remington's Pharmaceutical Sciences” 15th Edition, chapter 61, incorporate herein as a reference, for details on doses for administration.

4. Chlorambucil

Chlorambucil (also known as leukeran) was first synthesized by Everett et al. (1953). It is a bifunctional alkylating agent of the nitrogen mustard type that has been found active against selected human neoplastic diseases. Chlorambucil is known chemically as 4-[bis(2-chlorethyl)amino]benzenebutanoic acid.

Chlorambucil is available in tablet form for oral administration. It is rapidly and completely absorbed from the gastrointestinal tract. After single oral doses of 0.6-1.2 mg/kg, peak plasma chlorambucil levels are reached within one hour and the terminal half-life of the parent drug is estimated at 1.5 hours. 0.1 to 0.2 mg/kg/day or 3 to 6 mg/m²/day or alternatively 0.4 mg/kg may be used for antineoplastic treatment. Treatment regimes are well know to those of skill in the art and can be found in the “Physicians Desk Reference” and in “Remingtons Pharmaceutical Sciences” referenced herein.

Chlorambucil is indicated in the treatment of chronic lymphatic (lymphocytic) leukemia, malignant lymphomas including lymphosarcoma, giant follicular lymphoma and Hodgkin's disease. It is not curative in any of these disorders but may produce clinically useful palliation.

5. Busulfan

Busulfan (also known as myleran) is a bifunctional alkylating agent. Busulfan is known chemically as 1,4-butanediol dimethanesulfonate.

Busulfan is not a structural analog of the nitrogen mustards. Busulfan is available in tablet form for oral administration. Each scored tablet contains 2 mg busulfan and the inactive ingredients magnesium stearate and sodium chloride.

Busulfan is indicated for the palliative treatment of chronic myelogenous (myeloid, myelocytic, granulocytic) leukemia. Although not curative, busulfan reduces the total granulocyte mass, relieves symptoms of the disease, and improves the clinical state of the patient. Approximately 90% of adults with previously untreated chronic myelogenous leukemia will obtain hematologic remission with regression or stabilization of organomegaly following the use of busulfan. It has been shown to be superior to splenic irradiation with respect to survival times and maintenance of hemoglobin levels, and to be equivalent to irradiation at controlling splenomegaly.

6. Lomustine

Lomustine is one of the nitrosoureas used in the treatment of certain neoplastic diseases. It is 1-(2-chloro-ethyl)-3-cyclohexyl-1 nitrosourea. It is a yellow powder with the empirical formula of C₉H₁₆ClN₃O₂ and a molecular weight of 233.71. Lomustine is soluble in 10% ethanol (0.05 mg per mL) and in absolute alcohol (70 mg per mL). Lomustine is relatively insoluble in water (<0.05 mg per mL). It is relatively unionized at a physiological pH. Inactive ingredients in lomustine capsules are: magnesium stearate and mannitol.

Although it is generally agreed that lomustine alkylates DNA and RNA, it is not cross resistant with other alkylators. As with other nitrosoureas, it may also inhibit several key enzymatic processes by carbamoylation of amino acids in proteins.

Lomustine may be given orally. Following oral administration of radioactive lomustine at doses ranging from 30 mg/m² to 100 mg/m², about half of the radioactivity given was excreted in the form of degradation products within 24 hours.

The serum half-life of the metabolites ranges from 16 hours to 2 days. Tissue levels are comparable to plasma levels at 15 minutes after intravenous administration.

Lomustine has been shown to be useful as a single agent in addition to other treatment modalities, or in established combination therapy with other approved chemotherapeutic agents in both primary and metastatic brain tumors, in patients who have already received appropriate surgical and/or radiotherapeutic procedures. It has also proved effective in secondary therapy against Hodgkin's Disease in combination with other approved drugs in patients who relapse while being treated with primary therapy, or who fail to respond to primary therapy.

The recommended dose of lomustine in adults and children as a single agent in previously untreated patients is 130 mg/m² as a single oral dose every 6 weeks. In individuals with compromised bone marrow function, the dose should be reduced to 100 mg/m² every 6 weeks. When lomustine is used in combination with other myelosuppressive drugs, the doses should be adjusted accordingly. It is understood that other doses may be used for example, 20 mg/m² 30 mg/m², 40 mg/m², 50 mg/m², 60 mg/m², 70 mg/m², 80 mg/m², 90 mg/m², 100 mg/m², 120 mg/m² or any doses between these figures as determined by the clinician to be necessary for the individual being treated.

VIII. Nucleic Acid-Based Expression Systems

A. Vectors

The term “vector” is used to refer to a carrier nucleic acid molecule into which a nucleic acid sequence can be inserted for introduction into a cell where it can be replicated. A nucleic acid sequence can be “exogenous,” which means that it is foreign to the cell into which the vector is being introduced or that the sequence is homologous to a sequence in the cell but in a position within the host cell nucleic acid in which the sequence is ordinarily not found. Vectors include plasmids, cosmids, viruses (bacteriophage, animal viruses, and plant viruses), and artificial chromosomes (e.g., YACs). One of skill in the art would be well equipped to construct a vector through standard recombinant techniques, which are described in Maniatis et al., 1988 and Ausubel et al., 1994, both incorporated herein by reference.

The term “expression vector” refers to a vector containing a nucleic acid sequence coding for at least part of a gene product capable of being transcribed. In some cases, RNA molecules are then translated into a protein, polypeptide, or peptide. In other cases, these sequences are not translated, for example, in the production of antisense molecules or ribozymes. Expression vectors can contain a variety of “control sequences,” which refer to nucleic acid sequences necessary for the transcription and possibly translation of an operably linked coding sequence in a particular host organism. In addition to control sequences that govern transcription and translation, vectors and expression vectors may contain nucleic acid sequences that serve other functions as well and are described infra.

1. Promoters and Enhancers

A “promoter” is a control sequence that is a region of a nucleic acid sequence at which initiation and rate of transcription are controlled. It may contain genetic elements at which regulatory proteins and molecules may bind such as RNA polymerase and other transcription factors. The phrases “operatively positioned,” “operatively linked,” “under control,” and “under transcriptional control” mean that a promoter is in a correct functional location and/or orientation in relation to a nucleic acid sequence to control transcriptional initiation and/or expression of that sequence. A promoter may or may not be used in conjunction with an “enhancer,” which refers to a cis-acting regulatory sequence involved in the transcriptional activation of a nucleic acid sequence.

A promoter may be one naturally associated with a gene or sequence, as may be obtained by isolating the 5′ non-coding sequences located upstream of the coding segment and/or exon. Such a promoter can be referred to as “endogenous.” Similarly, an enhancer may be one naturally associated with a nucleic acid sequence, located either downstream or upstream of that sequence. Alternatively, certain advantages will be gained by positioning the coding nucleic acid segment under the control of a recombinant or heterologous promoter, which refers to a promoter that is not normally associated with a nucleic acid sequence in its natural environment. A recombinant or heterologous enhancer refers also to an enhancer not normally associated with a nucleic acid sequence in its natural environment. Such promoters or enhancers may include promoters or enhancers of other genes, and promoters or enhancers isolated from any other prokaryotic, viral, or eukaryotic cell, and promoters or enhancers not “naturally occurring,” i.e., containing different elements of different transcriptional regulatory regions, and/or mutations that alter expression. In addition to producing nucleic acid sequences of promoters and enhancers synthetically, sequences may be produced using recombinant cloning and/or nucleic acid amplification technology, including PCR™, in connection with the compositions disclosed herein (see U.S. Pat. No. 4,683,202; U.S. Pat. No. 5,928,906, each incorporated herein by reference). Furthermore, it is contemplated the control sequences that direct transcription and/or expression of sequences within non-nuclear organelles such as mitochondria, chloroplasts, and the like, can be employed as well.

Naturally, it will be important to employ a promoter and/or enhancer that effectively directs the expression of the DNA segment in the cell type, organelle, and organism chosen for expression. Those of skill in the art of molecular biology generally know the use of promoters, enhancers, and cell type combinations for protein expression, for example, see Sambrook et al. (1989), incorporated herein by reference. The promoters employed may be constitutive, tissue-specific, inducible, and/or useful under the appropriate conditions to direct high level expression of the introduced DNA segment, such as is advantageous in the large-scale production of recombinant proteins and/or peptides. The promoter may be heterologous or endogenous.

Table 4 lists several elements/promoters that may be employed, in the context of the present invention, to regulate the expression of a gene. This list is not intended to be exhaustive of all the possible elements involved in the promotion of expression but, merely, to be exemplary thereof. Table 5 provides examples of inducible elements, which are regions of a nucleic acid sequence that can be activated in response to a specific stimulus.

TABLE 4
Promoter and/or Enhancer

| Promoter/Enhancer | References |
|---|---|
| Immunoglobulin Heavy Chain | Banerji et al., 1983; Gilles et al., 1983; Grosschedl et al., |
| | 1985; Atchinson et al., 1986, 1987; Imler et al., 1987; |
| | Weinberger et al., 1984; Kiledjian et al., 1988; Porton et |
| | al.; 1990 |
| Immunoglobulin Light Chain | Queen et al., 1983; Picard et al., 1984 |
| T-Cell Receptor | Luria et al., 1987; Winoto et al., 1989; Redondo et al.; |
| | 1990 |
| HLA DQ a and/or DQ β | Sullivan et al., 1987 |
| β-Interferon | Goodbourn et al., 1986; Fujita et al., 1987; Goodbourn et |
| | al., 1988 |
| Interleukin-2 | Greene et al., 1989 |
| Interleukin-2 Receptor | Greene et al., 1989; Lin et al., 1990 |
| MHC Class II 5 | Koch et al., 1989 |
| MHC Class II HLA-DRa | Sherman et al., 1989 |
| β-Actin | Kawamoto et al., 1988; Ng et al.; 1989 |
| Muscle Creatine Kinase (MCK) | Jaynes et al., 1988; Horlick et al., 1989; Johnson et al., |
| | 1989 |
| Prealbumin (Transthyretin) | Costa et al., 1988 |
| Elastase I | Omitz et al., 1987 |
| Metallothionein (MTII) | Karin et al., 1987; Culotta et al., 1989 |
| Collagenase | Pinkert et al., 1987; Angel et al., 1987 |
| Albumin | Pinkert et al., 1987; Tranche et al., 1989, 1990 |
| α-Fetoprotein | Godbout et al., 1988; Campere et al., 1989 |
| t-Globin | Bodine et al., 1987; Perez-Stable et a.l, 1990 |
| β-Globin | Trudel et al., 1987 |
| c-fos | Cohen et al., 1987 |
| c-HA-ras | Triesman, 1986; Deschamps et al, 1985 |
| Insulin | Edlund et al., 1985 |
| Neural Cell Adhesion Molecule | Hirsh et al., 1990 |
| (NCAM) | |
| α₁-Antitrypain | Latimer et al., 1990 |
| H2B (TH2B) Histone | Hwang et al., 1990 |
| Mouse and/or Type I Collagen | Ripe et al., 1989 |
| Glucose-Regulated Proteins (GRP94 | Chang et al., 1989 |
| and GRP78) | |
| Rat Growth Hormone | Larsen et al., 1986 |
| Human Serum Amyloid A (SAA) | Edbrooke et al., 1989 |
| Troponin I (TN I) | Yutzey et al., 1989 |
| Platelet-Derived Growth Factor | Pech et al., 1989 |
| (PDGF) | |
| Duchenne Muscular Dystrophy | Klamut et al., 1990 |
| SV40 | Banerji et al., 1981; Moreau et al., 1981; Sleigh et al., |
| | 1985; Firak et al., 1986; Herr et al., 1986; Imbra et al., |
| | 1986; Kadesch et al., 1986; Wang et al., 1986; Ondek et |
| | al., 1987; Kuhl et al., 1987; Schaffner et al., 1988 |
| Polyoma | Swartzendruber et al., 1975; Vasseur et al., 1980; Katinka |
| | et al., 1980, 1981; Tyndell et al., 1981; Dandolo et al., |
| | 1983; de Villiers et al., 1984; Hen et al., 1986; Satake et |
| | al., 1988; Campbell and Villarreal, 1988 |
| Retroviruses | Kriegler et al., 1982, 1983; Levinson et al., 1982; Kriegler |
| | et al., 1983, 1984a, b, 1988; Bosze et al., 1986; Miksicek |
| | et al., 1986; Celander et al., 1987; Thiesen et al., 1988; |
| | Celander et al., 1988; Chol et al., 1988; Reisman et al., |
| | 1989 |
| Papilloma Virus | Campo et al., 1983; Lusky et al., 1983; Spandidos and |
| | Wilkie, 1983; Spalholz et al., 1985; Lusky et al., 1986; |
| | Cripe et al., 1987; Gloss et al., 1987; Hirochika et al., |
| | 1987; Stephens et al., 1987; Glue et al., 1988 |
| Hepatitis B Virus | Bulla et al., 1986; Jameel et al., 1986; Shaul et al., 1987; |
| | Spandau et al., 1988; Vannice et al., 1988 |
| Human Immunodeficiency Virus | Muesing et al., 1987; Hauber et al., 1988; Jakobovits et |
| | al., 1988; Feng et al., 1988; Takebe et al., 1988; Rosen et |
| | al., 1988; Berkhout et al., 1989; Laspia et al., 1989; Sharp |
| | et al., 1989; Braddock et al., 1989 |
| Cytomegalovirus (CMV) | Weber et al., 1984; Boshart et al., 1985; Foecking et al., |
| | 1986 |
| Gibbon Ape Leukemia Virus | Holbrook et al., 1987; Quinn et al., 1989 |

TABLE 5
Inducible Elements

| Element | Inducer | References |
|---|---|---|
| MT II | Phorbol Ester (TFA) | Palmiter et al., 1982; Haslinger et |
| | Heavy metals | al., 1985; Searle et al., 1985; |
| | | Stuart et al., 1985; Imagawa et |
| | | al., 1987, Karin et al., 1987; |
| | | Angel et al., 1987b; McNeall et |
| | | al., 1989 |
| MMTV (mouse mammary tumor | Glucocorticoids | Huang et al., 1981; Lee et al., |
| virus) | | 1981; Majors et al., 1983; |
| | | Chandler et al., 1983; Lee et al., |
| | | 1984; Ponta et al., 1985; Sakai et |
| | | al., 1988 |
| β-Interferon | poly(rI)x | Tavernier et al., 1983 |
| | poly(rc) | |
| Adenovirus 5 E2 | E1A | Imperiale et al., 1984 |
| Collagenase | Phorbol Ester (TPA) | Angel et al., 1987a |
| Stromelysin | Phorbol Ester (TPA) | Angel et al., 1987b |
| SV40 | Phorbol Ester (TPA) | Angel et al., 1987b |
| Murine MX Gene | Interferon, Newcastle Disease | Hug et al., 1988 |
| | Virus | |
| GRP78 Gene | A23187 | Resendez et al., 1988 |
| α-2-Macroglobulin | IL-6 | Kunz et al., 1989 |
| Vimentin | Serum | Rittling et al., 1989 |
| MHC Class I Gene H-2κb | Interferon | Blanar et al., 1989 |
| HSP70 | E1A, SV40 Large T | Taylor et al., 1989, 1990a, 1990b |
| | Antigen | |
| Proliferin | Phorbol Ester-TPA | Mordacq et al., 1989 |
| Tumor Necrosis Factor | PMA | Hensel et al., 1989 |
| Thyroid Stimulating Hormone α | Thyroid Hormone | Chatterjee et al., 1989 |
| Gene | | |

The identity of tissue-specific promoters or elements, as well as assays to characterize their activity, is well known to those of skill in the art. Examples of such regions include the human LIMK2 gene (Nomoto et al. 1999), the somatostatin receptor 2 gene (Kraus et al., 1998), murine epididymal retinoic acid-binding gene (Lareyre et al., 1999), human CD4 (Zhao-Emonet et al., 1998), mouse alpha2 (XI) collagen (Tsumaki, et al., 1998), D1A dopamine receptor gene (Lee, et al., 1997), insulin-like growth factor II (Wu et al., 1997), human platelet endothelial cell adhesion molecule-1 (Almendro et al., 1996).

2. Initiation Signals and Internal Ribosome Binding Sites

A specific initiation signal also may be required for efficient translation of coding sequences. These signals include the ATG initiation codon or adjacent sequences. Exogenous translational control signals, including the ATG initiation codon, may need to be provided. One of ordinary skill in the art would readily be capable of determining this and providing the necessary signals. It is well known that the initiation codon must be “in-frame” with the reading frame of the desired coding sequence to ensure translation of the entire insert. The exogenous translational control signals and initiation codons can be either natural or synthetic. The efficiency of expression may be enhanced by the inclusion of appropriate transcription enhancer elements.

In certain embodiments of the invention, the use of internal ribosome entry sites (IRES) elements are used to create multigene, or polycistronic, messages. IRES elements are able to bypass the ribosome scanning model of 5′ methylated Cap dependent translation and begin translation at internal sites (Pelletier and Sonenberg, 1988). IRES elements from two members of the picornavirus family (polio and encephalomyocarditis) have been described (Pelletier and Sonenberg, 1988), as well an IRES from a mammalian message (Macejak and Sarnow, 1991). IRES elements can be linked to heterologous open reading frames. Multiple open reading frames can be transcribed together, each separated by an IRES, creating polycistronic messages. By virtue of the IRES element, each open reading frame is accessible to ribosomes for efficient translation. Multiple genes can be efficiently expressed using a single promoter/enhancer to transcribe a single message (see U.S. Pat. No. 5,925,565 and 5,935,819, herein incorporated by reference).

3. Multiple Cloning Sites

Vectors can include a multiple cloning site (MCS), which is a nucleic acid region that contains multiple restriction enzyme sites, any of which can be used in conjunction with standard recombinant technology to digest the vector. (See Carbonelli et al., 1999, Levenson et al., 1998, and Cocea, 1997, incorporated herein by reference.) “Restriction enzyme digestion” refers to catalytic cleavage of a nucleic acid molecule with an enzyme that functions only at specific locations in a nucleic acid molecule. Many of these restriction enzymes are commercially available. Use of such enzymes is widely understood by those of skill in the art. Frequently, a vector is linearized or fragmented using a restriction enzyme that cuts within the MCS to enable exogenous sequences to be ligated to the vector.

“Ligation” refers to the process of forming phosphodiester bonds between two nucleic acid fragments, which may or may not be contiguous with each other. Techniques involving restriction enzymes and ligation reactions are well known to those of skill in the art of recombinant technology.

4. Splicing Sites

Most transcribed eukaryotic RNA molecules will undergo RNA splicing to remove introns from the primary transcripts. Vectors containing genomic eukaryotic sequences may require donor and/or acceptor splicing sites to ensure proper processing of the transcript for protein expression. (See Chandler et al., 1997, herein incorporated by reference.)

5. Polyadenylation Signals

In expression, one will typically include a polyadenylation signal to effect proper polyadenylation of the transcript. The nature of the polyadenylation signal is not believed to be crucial to the successful practice of the invention, and/or any such sequence may be employed. Preferred embodiments include the SV40 polyadenylation signal and/or the bovine growth hormone polyadenylation signal, convenient and/or known to function well in various target cells. Also contemplated as an element of the expression cassette is a transcriptional termination site. These elements can serve to enhance message levels and/or to minimize read through from the cassette into other sequences.

6. Origins of Replication

In order to propagate a vector in a host cell, it may contain one or more origins of replication sites (often termed “ori”), which is a specific nucleic acid sequence at which replication is initiated. Alternatively an autonomously replicating sequence (ARS) can be employed if the host cell is yeast.

7. Selectable and Screenable Markers

In certain embodiments of the invention, the cells contain nucleic acid construct of the present invention, a cell may be identified in vitro or in vivo by including a marker in the expression vector. Such markers would confer an identifiable change to the cell permitting easy identification of cells containing the expression vector. Generally, a selectable marker is one that confers a property that allows for selection. A positive selectable marker is one in which the presence of the marker allows for its selection, while a negative selectable marker is one in which its presence prevents its selection. An example of a positive selectable marker is a drug resistance marker.

Usually the inclusion of a drug selection marker aids in the cloning and identification of transformants, for example, genes that confer resistance to neomycin, puromycin, hygromycin, DHFR, GPT, zeocin and histidinol are useful selectable markers. In addition to markers conferring a phenotype that allows for the discrimination of transformants based on the implementation of conditions, other types of markers including screenable markers such as GFP, whose basis is colorimetric analysis, are also contemplated. Alternatively, screenable enzymes such as herpes simplex virus thymidine kinase (tk) or chloramphenicol acetyltransferase (CAT) may be utilized. One of skill in the art would also know how to employ immunologic markers, possibly in conjunction with FACS analysis. The marker used is not believed to be important, so long as it is capable of being expressed simultaneously with the nucleic acid encoding a gene product. Further examples of selectable and screenable markers are well known to one of skill in the art.

B. Host Cells

As used herein, the terms “cell,” “cell line,” and “cell culture” may be used interchangeably. All of these term also include their progeny, which is any and all subsequent generations. It is understood that all progeny may not be identical due to deliberate or inadvertent mutations. In the context of expressing a heterologous nucleic acid sequence, “host cell” refers to a prokaryotic or eukaryotic cell, and it includes any transformable organisms that is capable of replicating a vector and/or expressing a heterologous gene encoded by a vector. A host cell can, and has been, used as a recipient for vectors. A host cell may be “transfected” or “transformed,” which refers to a process by which exogenous nucleic acid is transferred or introduced into the host cell. A transformed cell includes the primary subject cell and its progeny.

Host cells may be derived from prokaryotes or eukaryotes, depending upon whether the desired result is replication of the vector or expression of part or all of the vector-encoded nucleic acid sequences. Numerous cell lines and cultures are available for use as a host cell, and they can be obtained through the American Type Culture Collection (ATCC), which is an organization that serves as an archive for living cultures and genetic materials (www.atcc.org). An appropriate host can be determined by one of skill in the art based on the vector backbone and the desired result. A plasmid or cosmid, for example, can be introduced into a prokaryote host cell for replication of many vectors. Bacterial cells used as host cells for vector replication and/or expression include DH5α, JM109, and KC8, as well as a number of commercially available bacterial hosts such as SURE® Competent Cells and SOLOPACK™ Gold Cells (STRATAGENE®, La Jolla). Alternatively, bacterial cells such as E. Coli LE392 could be used as host cells for phage viruses.

Examples of eukaryotic host cells for replication and/or expression of a vector include HeLa, NIH3T3, Jurkat, 293, Cos, CHO, Saos, and PC12. Many host cells from various cell types and organisms are available and would be known to one of skill in the art. Similarly, a viral vector may be used in conjunction with either a eukaryotic or prokaryotic host cell, particularly one that is permissive for replication or expression of the vector.

Some vectors may employ control sequences that allow it to be replicated and/or expressed in both prokaryotic and eukaryotic cells. One of skill in the art would further understand the conditions under which to incubate all of the above described host cells to maintain them and to permit replication of a vector. Also understood and known are techniques and conditions that would allow large-scale production of vectors, as well as production of the nucleic acids encoded by vectors and their cognate polypeptides, proteins, or peptides.

C. Expression Systems

Numerous expression systems exist that comprise at least a part or all of the compositions discussed above. Prokaryote- and/or eukaryote-based systems can be employed for use with the present invention to produce nucleic acid sequences, or their cognate polypeptides, proteins and peptides. Many such systems are commercially and widely available.

The insect cell/baculovirus system can produce a high level of protein expression of a heterologous nucleic acid segment, such as described in U.S. Pat. No. 5,871,986, 4,879,236, both herein incorporated by reference, and which can be bought, for example, under the name MAXBAC® 2.0 from INVITROGEN® and BACPAcK™ BACULOVIRUS EXPRESSION SYSTEM FROM CLONTECH®.

Other examples of expression systems include STRATAGENE®'S COMPLETE CONTROL™ Inducible Mammalian Expression System, which involves a synthetic ecdysone-inducible receptor, or its pET Expression System, an E. coli expression system. Another example of an inducible expression system is available from INVITROGEN®, which carries the T-REX™ (tetracycline-regulated expression) System, an inducible mammalian expression system that uses the full-length CMV promoter. INVITROGEN® also provides a yeast expression system called the Pichia methanolica Expression System, which is designed for high-level production of recombinant proteins in the methylotrophic yeast Pichia methanolica. One of skill in the art would know how to express a vector, such as an expression construct, to produce a nucleic acid sequence or its cognate polypeptide, protein, or peptide.

IX. Nucleic Acid Delivery

The general approach to the aspects of the present invention concerning compositions and/or therapeutics is to provide a cell with a genuine construct encoding a specific and/or desired protein, polypeptide and peptide, thereby permitting the desired activity of the proteins to take effect. While it is conceivable that the gene construct and/or protein may be delivered directly, a preferred embodiment involves providing a nucleic acid encoding a specific and desired protein, polypeptide and peptide to the cell. Following this provision, the proteinaceous composition is synthesized by the transcriptional and translational machinery of the cell, as well as any that may be provided by the expression construct. In providing antisense, ribozymes and other inhibitors, the preferred mode is also to provide a nucleic acid encoding the construct to the cell.

In certain embodiments of the invention, the nucleic acid encoding the gene may be stably integrated into the genome of the cell. In yet further embodiments, the nucleic acid may be stably maintained in the cell as a separate, episomal segment of DNA. Such nucleic acid segments and “episomes” encode sequences sufficient to permit maintenance and replication independent of and in synchronization with the host cell cycle. How the expression construct is delivered to a cell and/or where in the cell the nucleic acid remains is dependent on the type of expression construct employed.

A. DNA Delivery Using Viral Vectors 1

The ability of certain viruses to infect cells and enter cells via receptor- mediated endocytosis, and to integrate into host cell genome and/or express viral genes stably and/or efficiently have made them attractive candidates for the transfer of foreign genes into mammalian cells. Preferred gene therapy vectors of the present invention will generally be viral vectors.

Although some viruses that can accept foreign genetic material are limited in the number of nucleotides they can accommodate and/or in the range of cells they infect, these viruses have been demonstrated to successfully effect gene expression. However, adenoviruses do not integrate their genetic material into the host genome and/or therefore do not require host replication for gene expression, making them ideally suited for rapid, efficient, heterologous gene expression. Techniques for preparing replication-defective infective viruses are well known in the art.

Of course, in using viral delivery systems, one will desire to purify the virion sufficiently to render it essentially free of undesirable contaminants, such as defective interfering viral particles and endotoxins and other pyrogens such that it will not cause any untoward reactions in the cell, animal and/or individual receiving the vector construct. A preferred means of purifying the vector involves the use of buoyant density gradients, such as cesium chloride gradient centrifugation.

1. Adenoviral Vectors

A particular method for delivery of the expression constructs involves the use of an adenovirus expression vector. Although adenovirus vectors are known to have a low capacity for integration into genomic DNA, this feature is counterbalanced by the high efficiency of gene transfer afforded by these vectors. “Adenovirus expression vector” is meant to include those constructs containing adenovirus sequences sufficient to (a) support packaging of the construct and/or (b) to ultimately express a tissue and/or cell-specific construct that has been cloned therein.

The expression vector comprises a genetically engineered form of adenovirus. Knowledge of the genetic organization and adenovirus, a 36 kb, linear, double- stranded DNA virus, allows substitution of large pieces of adenoviral DNA with foreign sequences up to 7 kb (Grunhaus and Horwitz, 1992). In contrast to retrovirus, the adenoviral infection of host cells does not result in chromosomal integration because adenoviral DNA can replicate in an episomal manner without potential genotoxicity. Also, adenoviruses are structurally stable, and/or no genome rearrangement has been detected after extensive amplification.

Adenovirus is particularly suitable for use as a gene transfer vector because of its mid-sized genome, ease of manipulation, high titer, wide target-cell range and/or high infectivity. Both ends of the viral genome contain 100-200 base pair inverted repeats (ITRs), which are cis elements necessary for viral DNA replication and/or packaging. The early (E) and/or late (L) regions of the genome contain different transcription units that are divided by the onset of viral DNA replication. The E1 region (E1A and/or E1B) encodes proteins responsible for the regulation of transcription of the viral genome and/or a few cellular genes. The expression of the E2 region (E2A and/or E2B) results in the synthesis of the proteins for viral DNA replication. These proteins are involved in DNA replication, late gene expression and/or host cell shut-off (Renan, 1990). The products of the late genes, including the majority of the viral capsid proteins, are expressed only after significant processing of a single primary transcript issued by the major late promoter (MLP). The MLP (located at 16.8 m.u.) is particularly efficient during the late phase of infection, and/or all the mRNA's issued from this promoter possess a 5′-tripartite leader (TPL) sequence which makes them preferred mRNA's for translation.

In a current system, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and/or examine its genomic structure.

Generation and/or propagation of the current adenovirus vectors, which are replication deficient, depend on a unique helper cell line, designated 293, which was transformed from human embryonic kidney cells by Ad5 DNA fragments and constitutively expresses E1 proteins (E1A and/or E1B; Graham et al., 1977). Since the E3 region is dispensable from the adenovirus genome (Jones and Shenk, 1978), the current adenovirus vectors, with the help of 293 cells, carry foreign DNA in either the E1, the D3 and both regions (Graham and Prevec, 1991). Recently, adenoviral vectors comprising deletions in the E4 region have been described (U.S. Pat. No. 5,670,488, incorporated herein by reference).

In nature, adenovirus can package approximately 105% of the wild-type genome (Ghosh-Choudhury et al., 1987), providing capacity for about 2 extra kb of DNA. Combined with the approximately 5.5 kb of DNA that is replaceable in the E1 and/or E3 regions, the maximum capacity of the current adenovirus vector is under 7.5 kb, and/or about 15% of the total length of the vector. More than 80% of the adenovirus viral genome remains in the vector backbone.

Helper cell lines may be derived from human cells such as human embryonic kidney cells, muscle cells, hematopoietic cells and other human embryonic mesenchymal and epithelial cells. Alternatively, the helper cells may be derived from the cells of other mammalian species that are permissive for human adenovirus. Such cells include, e.g., Vero cells and other monkey embryonic mesenchymal and/or epithelial cells. As stated above, the preferred helper cell line is 293.

Recently, Racher et al. (1995) disclosed improved methods for culturing 293 cells and/or propagating adenovirus. In one format, natural cell aggregates are grown by inoculating individual cells into 1 liter siliconized spinner flasks (Techne, Cambridge, UK) containing 100-200 ml of medium. Following stirring at 40 rpm, the cell viability is estimated with trypan blue. In another format, Fibra-Cel microcarriers (Bibby Sterlin, Stone, UK) (5 g/l) is employed as follows. A cell inoculum, resuspended in 5 ml of medium, is added to the carrier (50 ml) in a 250 ml Erlenmeyer flask and/or left stationary, with occasional agitation, for 1 to 4 h. The medium is then replaced with 50 ml of fresh medium and/or shaking initiated. For virus production, cells are allowed to grow to about 80% confluence, after which time the medium is replaced (to 25% of the final volume) and/or adenovirus added at an MOI of 0.05. Cultures are left stationary overnight, following which the volume is increased to 100% and/or shaking commenced for another 72 h.

Other than the requirement that the adenovirus vector be replication defective, and at least conditionally defective, the nature of the adenovirus vector is not believed to be crucial to the successful practice of the invention. The adenovirus may be of any of the 42 different known serotypes and subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

As stated above, the typical vector according to the present invention is replication defective and will not have an adenovirus E1 region. Thus, it will be most convenient to introduce the transforming construct at the position from which the E1-coding sequences have been removed. However, the position of insertion of the construct within the adenovirus sequences is not critical to the invention. The polynucleotide encoding the gene of interest may also be inserted in lieu of the deleted E3 region in E3 replacement vectors as described by Karlsson et al. (1986) and in the E4 region where a helper cell line and helper virus complements the E4 defect.

Adenovirus growth and/or manipulation is known to those of skill in the art, and/or exhibits broad host range in vitro and in vivo. This group of viruses can be obtained in high titers, e.g. 10⁹ to 10¹¹ plaque-forming units per ml, and they are highly infective. The life cycle of adenovirus does not require integration into the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and/or therapeutic potential as in vivo gene transfer vectors.

Adenovirus vectors have been used in eukaryotic gene expression (Levrero et al., 1991; Gomez-Foix et al., 1992) and vaccine development (Grunhaus and Horwitz, 1992; Graham and Prevec, 1992). Recently, animal studies suggested that recombinant adenovirus could be used for gene therapy (Stratford-Perricaudet and Perricaudet, 1991a; Stratford-Perricaudet et al., 1991b; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and/or stereotactic inoculation into the brain (Le Gal La Salle et al., 1993). Recombinant adenovirus and adeno-associated virus (see below) can both infect and transduce non-dividing human primary cells.

2. AAV Vectors

Adeno-associated virus (AAV) is an attractive vector system for use in the cell transduction of the present invention as it has a high frequency of integration and it can infect nondividing cells, thus making it useful for delivery of genes into mammalian cells, for example, in tissue culture (Muzyczka, 1992) and in vivo. AAV has a broad host range for infectivity (Tratschin et al., 1984; Laughlin et al., 1986; Lebkowski et al., 1988; McLaughlin et al., 1988). Details concerning the generation and use of rAAV vectors are described in U.S. Pat. No. 5,139,941 and/or U.S. Pat. No. 4,797,368, each incorporated herein by reference.

Studies demonstrating the use of AAV in gene delivery include LaFace et al. (1988); Zhou et al. (1993); Flotte et al. (1993); and Walsh et al. (1994). Recombinant AAV vectors have been used successfully for in vitro and/or in vivo transduction of marker genes (Kaplitt et al., 1994; Lebkowski et al., 1988; Samulski et al., 1989; Yoder et al., 1994; Zhou et al., 1994; Hermonat and Muzyczka, 1984; Tratschin et al., 1985; McLaughlin et al., 1988) and genes involved in human diseases (Flotte et al., 1992; Luo et al., 1994; Ohi et al., 1990; Walsh et al., 1994; Wei et al., 1994). Recently, an AAV vector has been approved for phase I human trials for the treatment of cystic fibrosis.

AAV is a dependent parvovirus in that it requires coinfection with another virus (either adenovirus and a member of the herpes virus family) to undergo a productive infection in cultured cells (Muzyczka, 1992). In the absence of coinfection with helper virus, the wild type AAV genome integrates through its ends into human chromosome 19 where it resides in a latent state as a provirus (Kotin et al., 1990; Samulski et al., 1991). rAAV, however, is not restricted to chromosome 19 for integration unless the AAV Rep protein is also expressed (Shelling and Smith, 1994). When a cell carrying an AAV provirus is superinfected with a helper virus, the AAV genome is “rescued” from the chromosome and from a recombinant plasmid, and/or a normal productive infection is established (Samulski et al., 1989; McLaughlin et al., 1988; Kotin et al., 1990; Muzyczka, 1992).

Typically, recombinant AAV (rAAV) virus is made by cotransfecting a plasmid containing the gene of interest flanked by the two AAV terminal repeats (McLaughlin et al., 1988; Samulski et al., 1989; each incorporated herein by reference) and/or an expression plasmid containing the wild type AAV coding sequences without the terminal repeats, for example pIM45 (McCarty et al., 1991; incorporated herein by reference). The cells are also infected and transfected with adenovirus and plasmids carrying the adenovirus genes required for AAV helper function. rAAV virus stocks made in such fashion are contaminated with adenovirus which must be physically separated from the rAAV particles (for example, by cesium chloride density centrifugation). Alternatively, adenovirus vectors containing the AAV coding regions and cell lines containing the AAV coding regions and some and all of the adenovirus helper genes could be used (Yang et al., 1994; Clark et al., 1995). Cell lines carrying the rAAV DNA as an integrated provirus can also be used (Flotte et al., 1995).

3. Retroviral Vectors

Retroviruses have promise as gene delivery vectors due to their ability to integrate their genes into the host genome, transferring a large amount of foreign genetic material, infecting a broad spectrum of species and cell types and of being packaged in special cell-lines (Miller, 1992).

The retroviruses are a group of single-stranded RNA viruses characterized by an ability to convert their RNA to double-stranded DNA in infected cells by a process of reverse-transcription (Coffin, 1990). The resulting DNA then stably integrates into cellular chromosomes as a provirus and/or directs synthesis of viral proteins. The integration results in the retention of the viral gene sequences in the recipient cell and/or its descendants. The retroviral genome contains three genes, gag, pol, and/or env that code for capsid proteins, polymerase enzyme, and envelope components, respectively. A sequence found upstream from the gag gene contains a signal for packaging of the genome into virions. Two long terminal repeat (LTR) sequences are present at the 5′ and 3′ ends of the viral genome. These contain strong promoter and enhancer sequences and are also required for integration in the host cell genome (Coffin, 1990).

In order to construct a retroviral vector, a nucleic acid encoding a gene of interest is inserted into the viral genome in the place of certain viral sequences to produce a virus that is replication-defective. In order to produce virions, a packaging cell line containing the gag, pol, and env genes but without the LTR and packaging components is constructed (Mann et al., 1983). When a recombinant plasmid containing a cDNA, together with the retroviral LTR and packaging sequences is introduced into this cell line (by calcium phosphate precipitation for example), the packaging sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and/or stable expression require the division of host cells (Paskind et al., 1975).

Concern with the use of defective retrovirus vectors is the potential appearance of wild-type replication-competent virus in the packaging cells. This can result from recombination events in which the intact sequence from the recombinant virus inserts upstream from the gag, pol, env sequence integrated in the host cell genome. However, new packaging cell lines are now available that should greatly decrease the likelihood of recombination (Markowitz et al., 1988; Hersdorffer et al., 1990).

Gene delivery using second generation retroviral vectors has been reported. Kasahara et al. (1994) prepared an engineered variant of the Moloney murine leukemia virus, that normally infects only mouse cells, and modified an envelope protein so that the virus specifically bound to, and infected, human cells bearing the erythropoietin (EPO) receptor. This was achieved by inserting a portion of the EPO sequence into an envelope protein to create a chimeric protein with a new binding specificity.

B. Other Viral Vectors

Other viral vectors may be employed as expression constructs in the present invention. Vectors derived from viruses such as vaccinia virus (Ridgeway, 1988; Baichwal and Sugden, 1986; Coupar et al., 1988), sindbis virus, cytomegalovirus and/or herpes simplex virus may be employed. They offer several attractive features for various mammalian cells (Friedmann, 1989; Ridgeway, 1988; Baichwal and Sugden, 1986; Coupar et al., 1988; Horwich et al., 1990).

With the recent recognition of defective hepatitis B viruses, new insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. Chang et al. recently introduced the chloramphenicol acetyltransferase (CAT) gene into duck hepatitis B virus genome in the place of the polymerase, surface, and/or pre-surface coding sequences. It was cotransfected with wild-type virus into an avian hepatoma cell line. Culture media containing high titers of the recombinant virus were used to infect primary duckling hepatocytes. Stable CAT gene expression was detected for at least 24 days after transfection (Chang et al., 1991).

In certain further embodiments, the gene therapy vector will be HSV. A factor that makes HSV an attractive vector is the size and organization of the genome. Because HSV is large, incorporation of multiple genes and expression cassettes is less problematic than in other smaller viral systems. In addition, the availability of different viral control sequences with varying performance (temporal, strength, etc.) makes it possible to control expression to a greater extent than in other systems. It also is an advantage that the virus has relatively few spliced messages, further easing genetic manipulations. HSV also is relatively easy to manipulate and/or can be grown to high titers. Thus, delivery is less of a problem, both in terms of volumes needed to attain sufficient MOI and in a lessened need for repeat dosings.

C. Modified Viruses

In still further embodiments of the present invention, the nucleic acids to be delivered are housed within an infective virus that has been engineered to express a specific binding ligand. The virus particle will thus bind specifically to the cognate receptors of the target cell and deliver the contents to the cell. A novel approach designed to allow specific targeting of retrovirus vectors was recently developed based on the chemical modification of a retrovirus by the chemical addition of lactose residues to the viral envelope. This modification can permit the specific infection of hepatocytes via sialoglycoprotein receptors.

Another approach to targeting of recombinant retroviruses was designed in which biotinylated antibodies against a retroviral envelope protein and/or against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

D. Other Methods of DNA Delivery

In various embodiments of the invention, DNA is delivered to a cell as an expression construct. In order to effect expression of a gene construct, the expression construct must be delivered into a cell. As described herein, the preferred mechanism for delivery is via viral infection, where the expression construct is encapsidated in an infectious viral particle. However, several non-viral methods for the transfer of expression constructs into cells also are contemplated by the present invention. In one embodiment of the present invention, the expression construct may consist only of naked recombinant DNA and/or plasmids. Transfer of the construct may be performed by any of the methods mentioned which physically and/or chemically permeabilize the cell membrane. Some of these techniques may be successfully adapted for in vivo and/or ex vivo use, as discussed below.

1. Liposome-Mediated Transfection

In a further embodiment of the invention, the expression construct may be entrapped in a liposome. Liposomes are vesicular structures characterized by a phospholipid bilayer membrane and/or an inner aqueous medium. Multilamellar liposomes have multiple lipid layers separated by aqueous medium. They form spontaneously when phospholipids are suspended in an excess of aqueous solution. The lipid components undergo self- rearrangement before the formation of closed structures and/or entrap water and/or dissolved solutes between the lipid bilayers (Ghosh and Bachhawat, 1991). Also contemplated is an expression construct complexed with Lipofectamine (Gibco BRL).

Liposome-mediated nucleic acid delivery and expression of foreign DNA in vitro has been very successful (Nicolau and Sene, 1982; Fraley et al., 1979; Nicolau et al., 1987). Wong et al. (1980) demonstrated the feasibility of liposome-mediated delivery and/or expression of foreign DNA in cultured chick embryo, HeLa and hepatoma cells.

In certain embodiments of the invention, the liposome may be complexed with a hemagglutinating virus (HVJ). This has been shown to facilitate fusion with the cell membrane and/or promote cell entry of liposome-encapsulated DNA (Kaneda et al., 1989). In other embodiments, the liposome may be complexed and/or employed in conjunction with nuclear non-histone chromosomal proteins (HMG-1) (Kato et al., 1991). In yet further embodiments, the liposome may be complexed and/or employed in conjunction with both HVJ and HMG-1. In other embodiments, the delivery vehicle may comprise a ligand and a liposome. Where a bacterial promoter is employed in the DNA construct, it also will be desirable to include within the liposome an appropriate bacterial polymerase.

As described in U.S. Pat. No. 5,641,484, liposomes are particularly well suited for the treatment of HER2/neu-mediated cancer.

a. Preparation of liposomes

Catatonic liposomes which are efficient transfection reagents for p21^{Cip1/WAF1} for animal cells can be prepared using the method of Gao et al. (1991). Gao et al. describes a novel catatonic cholesterol derivative that can be synthesized in a single step. Liposomes made of this lipid are reportedly more efficient in transfection and less toxic to treated cells than those made with the reagent Lipofectin. These lipids are a mixture of DC- Chol (“3β(N-(N′N′-dimethylaminoethane)-carbamoyl cholesterol”) and DOPE (“dioleoylphosphatidylethanolamine”). The steps in producing these liposomes are as follows.

DC-Chol is synthesized by a simple reaction from cholesteryl chloroformate and N,N-Dimethylethylenediamine. A solution of cholesteryl chloroformate (2.25 g, 5 mmol in 5 ml dry chloroform) is added dropwise to a solution of excess N,N- Dimethylethylenediamine (2 ml, 18.2 mmol in 3 ml dry chloroform) at 0° C. Following removal of the solvent by evaporation, the residue is purified by recrystallization in absolute ethanol at 4° C. and dried in vacuo. The yield is a white powder of DC-Chol.

Cationic liposomes are prepared by mixing 1.2 μmol of DC-Chol and 8.0 μmol of DOPE in chloroform. This mixture is then dried, vacuum desiccated, and resuspended in 1 ml sterol 20 mM Hepes buffer (pH 7.8) in a tube. After 24 hours of hydration at 4° C., the dispersion is sonicated for 5-10 minutes in a sonicator form liposomes with an average diameter of 150-200 nm.

To prepare a liposome/DNA complex, the inventors use the following steps. The DNA to be transfected is placed in DMEM/F12 medium in a ratio of 15 μg DNA to 50.mu.1 DMEM/F12. DMEM/F12 is then used to dilute the DC-Chol/DOPE liposome mixture to a ratio of 50 μl DMEZM/F12 to 100.mu.1 liposome. The DNA dilution and the liposome dilution are then gently mixed, and incubated at 37° C. for 10 minutes. Following incubation, the DNA/liposome complex is ready for injection.

b. In Vivo Treatment of HER2/neu-Mediated Cancer Via Liposomes

U.S. Pat. No. 5,641,484, incorporated in its entirety by reference herein, teaches that liposome-mediated direct gene transfer techniques can be employed to obtain suppression of HER2/neu-overexpressing human cancer cells in living host. The protocol for described therein was as follows.

Female nude mice (5-6 weeks old) were given intraperitoneal injections of SK-OV-3 cells (2×10⁶/100 μl). SK-OV-3 cells are human ovarian cancer cells that have been shown to grow within the peritoneal cavity of nude mice. After five days, the mice were given intraperitoneal injections of various compounds. Some mice were injected with the therapeutic DNA alone, some were injected with liposome/therapeutic DNA complex prepared in the manner described above, and some were injected with liposome/mutant therapeutic DNA complex. 200 μl of a given compound was injected into a given mouse. After the initial injections, injections were repeated every seven days throughout the life of the mouse.

The results described therein indicate that liposome-mediated gene transfer can inhibit HER2/neu-overexpressing human ovarian cancer cell growth. Therefore, it is predictable that liposome-mediated p21^{Cip1WAF1} gene therapy may serve as a powerful therapeutic agent for HER-2 neu-overexpressing human ovarian cancers by direct targeting of p21^{Cip1WAF1} T145A at the HER-2 neu-oncogene.

c. Liposomal Transfection With p21^{Cip1WAF1} T145A to Treat Humans

Based on the results of the in vivo animal studies described in U.S. Pat. No. 5,641,484, those of skill in the art will understand and predict the enormous potential for human treatment of HER2/neu-mediated cancers with p21^{Cip1WAF1} T145A DNA complexed to liposomes. Clinical studies to demonstrate these affects are contemplated. Those of skill in the art will recognize that the best treatment regimens for using p21^{Cip1WAF1} T145A to suppress HER2/neu-mediated cancers can be straightforwardly determined. This is not a question of experimentation, but rather one of optimization, which is routinely conducted in the medical arts. In vivo studies in nude mice provide a starting point from which to begin to optimize the dosage and delivery regimes. The frequency of injection is initially once a week, as was done in the mice studies described in U.S. Pat. No. 5,641,484. However, this frequency might be optimally adjusted from one day to every two weeks to monthly, depending upon the results obtained from the initial clinical trials and the needs of a particular patient. Human dosage amounts can initially be determined by extrapolating from the amount of p21^{Cip1WAF1} T145A used in mice, approximately 15 μg of plasmid DNA per 50 g body weight. Based on this, a 50 kg woman would require treatment with 15 mg of DNA per dose. Of course, this dosage amount may be adjusted upward or downward, as is routinely done in such treatment protocols, depending on the results of the initial clinical trials and the needs of a particular patient. These clinical trials are anticipated to show utility of p21^{Cip1WAF1} T145A and other neu-suppressing gene products for the treatment of HER2/neu-overexpressing cancers in humans. Dosage and frequency regimes will initially be based on the data obtained from in vivo animal studies, as is done frequently in the art. 2. Electroporation

In certain embodiments of the present invention, the expression construct is introduced into the cell via electroporation. Electroporation involves the exposure of a suspension of cells and/or DNA to a high-voltage electric discharge.

Transfection of eukaryotic cells using electroporation has been quite successful. Mouse pre-B lymphocytes have been transfected with humankappa- immunoglobulin genes (Potter et al., 1984), and/or rat hepatocytes have been transfected with the chloramphenicol acetyltransferase gene (Tur-Kaspa et al., 1986) in this manner.

3. Calcium Phosphate and/or DEAE-Dextran

In other embodiments of the present invention, the expression construct is introduced to the cells using calcium phosphate precipitation. HumanKB cells have been transfected with adenovirus 5 DNA (Graham and Van Der Eb, 1973) using this technique. Also in this manner, mouse L(A9), mouse C127, CHO, CV-1, BHK, NIH3T3 and/or HeLa cells were transfected with a neomycin marker gene (Chen and Okayama, 1987), and/or rat hepatocytes were transfected with a variety of marker genes (Rippe et al., 1990).

In another embodiment, the expression construct is delivered into the cell using DEAE-dextran followed by polyethylene glycol. In this manner, reporter plasmids were introduced into mouse myeloma and/or erythroleukemia cells (Gopal, 1985).

4. Particle Bombardment

Another embodiment of the invention for transferring a naked DNA expression construct into cells may involve particle bombardment. This method depends on the ability to accelerate DNA-coated microprojectiles to a high velocity allowing them to pierce cell membranes and/or enter cells without killing them (Klein et al., 1987). Several devices for accelerating small particles have been developed. One such device relies on a high voltage discharge to generate an electrical current, which in turn provides the motive force (Yang et al, 1990). The microprojectiles used have consisted of biologically inert substances such as tungsten and/or gold beads.

5. Direct Microinjection and/or Sonication Loading

Further embodiments of the present invention include the introduction of the expression construct by direct microinjection and/or sonication loading. Direct microinjection has been used to introduce nucleic acid constructs into Xenopus oocytes (Harland and Weintraub, 1985), and/or LTK-fibroblasts have been transfected with the thymidine kinase gene by sonication loading (Fechheimer et al., 1987).

6. Adenoviral Assisted Transfection

In certain embodiments of the present invention, the expression construct is introduced into the cell using adenovirus assisted transfection. Increased transfection efficiencies have been reported in cell systems using adenovirus coupled systems (Kelleher and Vos, 1994; Cotten et al., 1992; Curiel, 1994).

X. In vivo Delivery and Treatment Protocols

Where the gene itself is employed to introduce the gene products, a convenient method of introduction will be through the use of a recombinant vector which incorporates the desired gene, together with its associated control sequences. The preparation of recombinant vectors is well known to those of skill in the art and described in many references, such as, for example, Sambrook et al. (1989), specifically incorporated herein by reference.

In vectors, it is understood that the DNA coding sequences to be expressed, in this case those encoding the neu-suppressing gene products, are positioned adjacent to and under the control of a promoter. It is understood in the art that to bring a coding sequence under the control of such a promoter, one generally positions the 5′ end of the transcription initiation site of the transcriptional reading frame of the gene product to be expressed between about 1 and about 50 nucleotides “downstream” of (i.e., 3′ of) the chosen promoter. One may also desire to incorporate into the transcriptional unit of the vector an appropriate polyadenylation site (e.g., 5′-AATAAA-3′), if one was not contained within the original inserted DNA. Typically, these poly A addition sites are placed about 30 to 2000 nucleotides “downstream” of the coding sequence at a position prior to transcription termination.

While use of the control sequences of p21 will be preferred, there is no reason why other control sequences could not be employed, so long as they are compatible with the genotype of the cell being treated. Thus, one may mention other useful promoters by way of example, including, e.g., an SV40 early promoter, a long terminal repeat promoter from retrovirus, an actin promoter, a heat shock promoter, a metallothionein promoter, and the like.

For introduction of the nucleic acid encoding the mutant form of p21^{Cip1/WAF1}, it is proposed that one will desire to preferably employ a vector construct that will deliver the desired gene to the affected cells. This will, of course, generally require that the construct be delivered to the targeted tumor cells, for example, breast, genital, or lung tumor cells. It is proposed that this may be achieved most preferably by introduction of the desired gene through the use of a viral or non viral vectors to carry the p21 sequences to efficiently transfect the tumor, or pretumorous tissue. This infection may be achieved preferably by liposomal delivery but may also be via adenoviral, a retroviral, a vaccinia viral vector or adeno-associated virus.

These vectors have been successfully used to deliver desired sequences to cells and tend to have a high infection efficiency.

Commonly used viral promoters for expression vectors are derived from polyoma, cytomegalovirus, Adenovirus 2, and Simian Virus 40 (SV40). The early and late promoters of SV40 virus are particularly useful because both are obtained easily from the virus as a fragment which also contains the SV40 viral origin of replication. Smaller or larger SV40 fragments may also be used, provided there is included the approximately 250 bp sequence extending from the HindIII site toward the BglI site located in the viral origin of replication. Further, it is also possible, and often desirable, to utilize promoter or control sequences normally associated with the desired gene sequence, provided such control sequences are compatible with the host cell systems.

The origin of replication may be provided either by construction of the vector to include an exogenous origin, such as may be derived from SV40 or other viral (e.g., Polyoma, Adeno, VSV, BPV) source, or may be provided by the host cell chromosomal replication mechanism. If the vector is integrated into the host cell chromosome, the latter is often sufficient.

A. Liposomal Transfection

Thus the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers (Ghosh and Bachhawat, 1991). Also contemplated are lipofectamine-DNA complexes.

The present invention also provides particularly useful methods for introducing neu-suppressing gene products into cells. One method of in vivo gene transfer which can lead to expression of genes transfected into cells involves the use of liposomes. Liposomes can be used for both in vitro and in vivo transfection. Liposome-mediated gene transfer seems to have great potential for certain in vivo applications in animals (Nicolau et al., 1987). Studies have shown that intravenously injected liposomes are taken up essentially in the liver and the spleen, by the macrophages of the reticuloendothelial system. The specific cellular sites of uptake of injected liposomes appears to be mainly spleen macrophages and liver Kupffer cells. Intravenous injection of liposomes/DNA complexes can lead to the uptake of DNA by these cellular sites, and result in the expression of a gene product encoded in the DNA (Nicolau, 1982).

The inventors contemplate that neu-suppressing gene products can be introduced into cells using liposome-mediated gene transfer. It is proposed that such constructs can be coupled with liposomes and directly introduced via a catheter, as described by Nabel et al. (1990). By employing these methods, the neu-suppressing gene products can be expressed efficiently at a specific site in vivo, not just the liver and spleen cells which are accessible via intravenous injection. Therefore, this invention also encompasses compositions of DNA constructs encoding a neu-suppressing gene product formulated as a DNA/liposome complex and methods of using such constructs.

Liposomal transfection can be via liposomes composed of, for example, phosphatidylcholine (PC), phosphatidylserine (PS), cholesterol (Chol), N-[1-(2,3-dioleyloxy)propyl]-N,N-trimethylammonium chloride (DOTMA), dioleoylphosphatidylethanolamine (DOPE), and/or 3 beta.[N-(N′N′-dimethylaminoethane)- carbarmoyl cholesterol (DC-Chol), as well as other lipids known to those of skill in the art. Those of skill in the art will recognize that there are a variety of liposomal transfection techniques which will be useful in the present invention. Among these techniques are those described in Nicolau et al., 1987, Nabel et al., 1990, and Gao et al., 1991. In a specific embodiment, the liposomes comprise DC-Chol. More particularly, the inventors the liposomes comprise DC-Chol and DOPE which have been prepared following the teaching of Gao et al. (1991) in the manner described in the Preferred Embodiments Section. The inventors also anticipate utility for liposomes comprised of DOTMA, such as those which are available commercially under the trademark Lipofectin™, from Vical, Inc., in San Diego, Calif.

Liposomes may be introduced into contact with cells to be transfected by a variety of methods. In cell culture, the liposome-DNA complex can simply be dispersed in the cell culture solution. For application in vivo, liposome-DNA complex are typically injected. Intravenous injection allow liposome-mediated transfer of DNA complex, for example, the liver and the spleen. In order to allow transfection of DNA into cells which are not accessible through intravenous injection, it is possible to directly inject the liposome-DNA complexes into a specific location in an animal's body. For example, Nabel et al. teach injection via a catheter into the arterial wall. In another example, the inventors have used intraperitoneal injection to allow for gene transfer into mice.

The present invention also contemplates compositions comprising a liposomal complex. This liposomal complex will comprise a lipid component and a DNA segment encoding a nucleic acid encoding a mutant form of p21^{Cip1/WAF1}. The nucleic acid encoding the mutant form of p21^{Cip1/WAF1} employed in the liposomal complex can be, for example, one which encodes p21-T145A or p21-T145D.

The lipid employed to make the liposomal complex can be any of the above-discussed lipids. In particular, DOTMA, DOPE, and/or DC-Chol may form all or part of the liposomal complex. The inventors have had particular success with complexes comprising DC-Chol. In a preferred embodiment, the lipid will comprise DC-Chol and DOPE. While any ratio of DC-Chol to DOPE is anticipated to have utility, it is anticipated that those comprising a ratio of DC-Chol:DOPE between 1:20 and 20:1 will be particularly advantageous. The inventors have found that liposomes prepared from a ratio of DC-Chol:DOPE of about 1:10 to about 1:5 have been useful.

In a specific embodiment, one employs the smallest region needed to enhance retention of p21 in the nucleus of a cell so that one is not introducing unnecessary DNA into cells which receive a p21 gene construct. Techniques well known to those of skill in the art, such as the use of restriction enzymes, will allow for the generation of small regions of p21. The ability of these regions to inhibit neu can easily be determined by the assays reported in the Examples.

In certain embodiments of the invention, the liposome may be complexed with a hemagglutinatin virus (HVJ). This has been shown to facilitate fusion with the cell membrane and promote cell entry of liposome-encapsulated DNA (Kaneda et al., 1989). In other embodiments, the liposome may be complexed or employed in conjunction with nuclear non-histone chromosomal proteins (HMG-1) (Kato et al., 1991). In yet further embodiments, the liposome may be complexed or employed in conjunction with both HVJ and HMG-1. In that such expression constructs have been successfully employed in transfer and expression of nucleic acid in vitro and in vivo, then they are applicable for the present invention. Where a bacterial promoter is employed in the DNA construct, it also will be desirable to include within the liposome an appropriate bacterial polymerase.

B. Adenovirus

Another method for in vivo delivery involves the use of an adenovirus vector. “Adenovirus expression vector” is meant to include those constructs containing adenovirus sequences sufficient to (a) support packaging of the construct and (b) to express an polynucleotide that has been cloned therein.

Adenovirus is a particularly suitable gene transfer vector because of its midsized genome, ease of manipulation, high titer, wide target-cell range and high infectivity. Both ends of the viral genome contain 100-200 base pair inverted repeats (ITRs), which are cis elements necessary for viral DNA replication and packaging. The early (E) and late (L) regions of the genome contain different transcription units that are divided by the onset of viral DNA replication. The E1 region (E1A and E1B) encodes proteins responsible for the regulation of transcription of the viral genome and a few cellular genes. The expression of the E2 region (E2A and E2B) results in the synthesis of the proteins for viral DNA replication. These proteins are involved in DNA replication, late gene expression and host cell shut-off (Renan, 1990). The products of the late genes, including the majority of the viral capsid proteins, are expressed only after significant processing of a single primary transcript issued by the major late promoter (MLP). The MLP, located at 16.8 m.mu. is particularly efficient during the late phase of infection, and all the mRNA's issued from this promoter possess a 5′-tripartite leader (TL) sequence which makes them preferred mRNA's for translation.

In some cases, recombinant adenovirus is generated from homologous recombination between shuttle vector and provirus vector. Due to the possible recombination between two proviral vectors, wild-type adenovirus may be generated from this process. Therefore, it is critical to isolate a single clone of virus from an individual plaque and examine its genomic structure. Use of the YAC system is an alternative approach for the production of recombinant adenovirus.

A particular method of introducing the mutant form of p21^{Cip1/WAF1} to an animal is to introduce a replication-deficient adenovirus containing the nucleic acid encoding the mutant form of p21^{Cip1/WAF1}. The replication-deficient construct made by E1B and E3 deletion also avoids the viral reproduction inside the cell and transfer to other cells and infection of other people, which means the viral infection activity is shut down after it infects the target cell. The nucleic acid encoding the mutant form of p21^{Cip1/WAF1} is still expressed inside the cells. Also, unlike retrovirus, which can only infect proliferating cells, adenovirus is able to transfer the nucleic acid encoding the mutant form of p21^{Cip1/WAF1} into both proliferating and non-proliferating cells. Further, the extrachromosomal location of adenovirus in the infected cells decreases the chance of cellular oncogene activation within the treated animal.

Introduction of the adenovirus containing the neu-suppressing gene product gene into a suitable host is typically done by injecting the virus contained in a buffer.

The nature of the adenovirus vector is not believed to be crucial to the successful practice of the invention. Of course, as discussed above, it is advantageous if the adenovirus vector is replication defective, or at least conditionally defective, The adenovirus may be of any of the 42 different known serotypes or subgroups A-F. Adenovirus type 5 of subgroup C is the preferred starting material in order to obtain the conditional replication-defective adenovirus vector for use in the present invention. This is because Adenovirus type 5 is a human adenovirus about which a great deal of biochemical and genetic information is known, and it has historically been used for most constructions employing adenovirus as a vector.

Adenovirus is easy to grow and manipulate and exhibits broad host range in vitro and in vivo. This group of viruses can be obtained in high titers, e.g., 10⁹-10¹¹ plaque-forming units per ml, and they are highly infective. The life cycle of adenovirus does not require integration in to the host cell genome. The foreign genes delivered by adenovirus vectors are episomal and, therefore, have low genotoxicity to host cells. No side effects have been reported in studies of vaccination with wild-type adenovirus (Couch et al., 1963; Top et al., 1971), demonstrating their safety and therapeutic potential as in vivo gene transfer vectors.

Adenovirus have been used in eukaryotic gene expression (Levrero et al., 1991; Gomez-Foix et al., 1992) and vaccine development (Grunhaus and Horwitz, 1992; Graham and Prevec, 1992). Animal studies have suggested that recombinant adenovirus could be used for gene therapy (Stratford-Perricaudet and Perricaudet, 1991; Stratford-Perricaudet et al., 1990; Rich et al., 1993). Studies in administering recombinant adenovirus to different tissues include trachea instillation (Rosenfeld et al., 1991; Rosenfeld et al., 1992), muscle injection (Ragot et al., 1993), peripheral intravenous injections (Herz and Gerard, 1993) and stereotatic inoculation into the brain (Le Gal La Salle et al., 1993).

C. Retroviruses

The retroviruses are a group of single-stranded RNA viruses characterized by an ability to convert their RNA to double-stranded DNA to infected cells by a process of reverse-transcription (Coffin, 1990). The resulting DNA then stably integrates into cellular chromosomes as a provirus and directs synthesis of viral proteins. The integration results in the retention of the viral gene sequences in the recipient cell and its descendants. The retroviral genome contains three genes, gag, pol, and env that code for capsid proteins, polymerase enzyme, and envelope components, respectively. A sequence found upstream from the gag gene, termed y components is constructed (Mann et al., 1983). When a recombinant plasmid containing a human cDNA, together with the retroviral LTR and ψ sequences is introduced into this cell line (by calcium phosphate precipitation for example), the ψ sequence allows the RNA transcript of the recombinant plasmid to be packaged into viral particles, which are then secreted into the culture media (Nicolas and Rubenstein, 1988; Temin, 1986; Mann et al., 1983). The media containing the recombinant retroviruses is then collected, optionally concentrated, and used for gene transfer. Retroviral vectors are able to infect a broad variety of cell types. However, integration and stable expression require the division of host cells (Paskind et al., 1975).

A novel approach designed to allow specific targeting of retrovirus vectors was developed based on the chemical modification of a retrovirus by the chemical addition of lactose residues to the viral envelope. This modification could permit the specific infection of hepatocytes via sialoglycoprotein receptors.

A different approach to targeting of recombinant retroviruses was designed in which biotinylated antibodies against a retroviral envelope protein and against a specific cell receptor were used. The antibodies were coupled via the biotin components by using streptavidin (Roux et al., 1989). Using antibodies against major histocompatibility complex class I and class II antigens, they demonstrated the infection of a variety of human cells that bore those surface antigens with an ecotropic virus in vitro (Roux et al., 1989).

There are certain limitations to the use of retrovirus vectors in all aspects of the present invention. For example, retrovirus vectors usually integrate into random sites in the cell genome. This can lead to insertional mutagenesis through the interruption of host genes or through the insertion of viral regulatory sequences that can interfere with the function of flanking genes (Varmus et al., 1981). Another concern with the use of defective retrovirus vectors is the potential appearance of wild-type replication-competent virus in the packaging cells. This can result from recombination events in which the intact ψ sequence from the recombinant virus inserts upstream from the gag, pol, env sequence integrated in the host cell genome. However, neu packaging cell lines are now available that should greatly decrease the likelihood of recombination (Markowitz et al., 1988; Hersdorffer et al., 1990).

One limitation to the use of retrovirus vectors in vivo is the limited ability to produce retroviral vector titers greater than 10⁶ infections U/mL. Titers 10- to 1,000-fold higher are necessary for many in vivo applications.

Several properties of the retrovirus have limited its use in lung cancer treatment (Stratford-Perricaudet and Perricaudet, 1991; (i) Infection by retrovirus depends on host cell division. In human cancer, very few mitotic cells can be found in tumor lesions. (ii) The integration of retrovirus into the host genome may cause adverse effects on target cells, because malignant cells are high in genetic instability. (iii) Retrovirus infection is often limited by a certain host range. (iv) Retrovirus has been associated with many malignancies in both mammals and vertebrates. (v) The titer of retrovirus, in general, is 100- to 1,000-fold lower than that of adenovirus.

D. Other Viral Vectors as Expression Constructs

Other viral vectors may be employed as expression constructs in the present invention. Vectors derived from viruses such as vaccinia virus (Ridgeway, 1988; Baichwal and Sugden, 1986; Coupar et al., 1988) adeno-associated virus (AAV) (Ridgeway, 1988; Baichwal and Sugden, 1986; Hermonat and Muzycska, 1984) and herpes viruses may be employed. They offer several attractive features for various mammalian cells (Friedmann, 1989; Ridgeway, 1988; Baichwal and Sugden, 1986; Coupar et al., 1988; Howrich et al., 1990).

With the recognition of defective hepatitis B viruses, neu insight was gained into the structure-function relationship of different viral sequences. In vitro studies showed that the virus could retain the ability for helper-dependent packaging and reverse transcription despite the deletion of up to 80% of its genome (Horwich et al., 1990). This suggested that large portions of the genome could be replaced with foreign genetic material. The hepatotropism and persistence (integration) were particularly attractive properties for liver-directed gene transfer. Chang et al. introduced the chloramphenicol acetyltransferase (CAT) gene into duck hepatitis B virus genome in the place of the polymerase, surface, and pre-surface coding sequences. It was cotransfected with wild-type virus into an avian hepatoma cell line. Cultures media containing high titers of the recombinant virus were used to infect primary duckling hepatocytes. Stable CAT gene expression was detected for at least 24 days after transfection (Chang et al., 1991).

E. Other non-viral vectors

In order to effect expression of sense or antisense gene constructs, the expression construct must be delivered into a cell. This delivery may be accomplished in vitro, as in laboratory procedures for transforming cells lines, or in vivo or ex vivo (see below), as in the treatment of certain disease states. As described above, delivery may be via viral infection where the expression construct is encapsidated in an infectious viral particle.

Several non-viral methods for the transfer of expression constructs into cultured mammalian cells also are contemplated by the present invention. These include calcium phosphate precipitation (Graham and Van Der Eb, 1973; Chen and Okayama, 1987; Rippe et al., 1990) DEAE-dextran (Gopal, 1985), electroporation (Tur-Kaspa et al., 1986; Potter et al., 1984). direct microinjection (Harland and Weintraub, 1985), DNA-loaded liposomes (Nicolau and Sene, 1982; Fraley et al., 1979) and lipofectamine-DNA complexes, cell sonication (Fechheimer et al., 1987), gene bombardment using high velocity microprojectiles (Yang et al., 1990), and receptor-mediated transfection (Wu and Wu, 1987; Wu and Wu, 1988). Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In one embodiment of the invention, the expression construct may simply consist of naked recombinant DNA or plasmids. Transfer of the construct may be performed by any of the methods mentioned above which physically or chemically permeabilize the cell membrane. This is particularly applicable for transfer permeabilize the cell membrane. This is particularly applicable for transfer in vitro but it may be applied to in vivo use as well. Dubensky et al. (1984) successfully injected polyomavirus DNA in the form of CaPO₄ precipitates into liver and spleen of adult and newborn mice demonstrating active viral replication and acute infection. Benvenisty and Neshif (1986) also demonstrated that direct intraperitoneal injection of CaPO₄ precipitated plasmids results in expression of the transfected genes. It is envisioned that DNA encoding a gene of interest may also be transferred in a similar manner in vivo and express the gene product.

Another embodiment of the invention for transferring a naked DNA expression construct into cells may involve particle bombardment. This method depends on the ability to accelerate DNA coated microprojectiles to a high velocity allowing them to pierce cell membranes and enter cells without killing them (Klein et al., 1987). Several devices for accelerating small particles have been developed. One such device relies on a high voltage discharge to generate an electrical current, which in turn provides the motive force (Yang et al., 1990). The microprojectiles used have consisted of biologically inert substances such as tungsten or gold beads.

Selected organs including the liver, skin, and muscle tissue of rats and mice have been bombarded in vivo (Yang et al, 1990; Zelenin et al., 1991). This may require surgical exposure of the tissue or cells, to eliminate any intervening tissue between the gun and the target organ, i.e., ex vivo treatment. Again, DNA encoding a particular gene may be delivered via this method and still be incorporated by the present invention.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (ASOR) (Wu and Wu, 1987) and transferrin (Wagner et al., 1990). A synthetic neoglycoprotein, which recognizes the same receptor as ASOR, has been used as a gene delivery vehicle (Ferkol et al., 1993; Perales et al., 1994) and epidermal growth factor (EGF) has also been used to deliver genes to squamous carcinoma cells (Myers, EPO 0273085).

In other embodiments, the delivery vehicle may comprise a ligand and a liposome. For example, Nicolau et al. (1987) employed lactosyl-ceramide, a galactose-terminal asialganglioside, incorporated into liposomes and observed an increase in the uptake of the insulin gene by hepatocytes. Thus, it is feasible that a nucleic acid encoding a particular gene also may be specifically delivered into a cell type such as lung, epithelial or tumor cells, by any number of receptor-ligand systems with or without liposomes. For example, epidermal growth factor (EGF) may be used as the receptor for mediated delivery of a nucleic acid encoding a gene in many tumor cells that exhibit upregulation of EGF receptor. Mannose can be used to target the mannose receptor on liver cells. Also, antibodies to CD5 (CLL), CD22 (lymphoma), CD25 (T-cell leukemia) and MAA (melanoma) can similarly be used as targeting moieties.

In certain embodiments, gene transfer may more easily be performed under ex vivo conditions. Ex vivo gene therapy refers to the isolation of cells from an animal, the delivery of a nucleic acid into the cells, in vitro, and then the return of the modified cells back into an animal. This may involve the surgical removal of tissue/organs from an animal or the primary culture of cells and tissues. Anderson et al., U.S. Pat. No. 5,399,346, and incorporated herein in its entirety, disclose ex vivo therapeutic methods.

XI. Pharmaceutical Preparations

Pharmaceutical compositions of the present invention comprise an effective amount of one or more forms of mutant p21^{Cip1/WAF1} or additional agent dissolved or dispersed in a pharmaceutically acceptable carrier or excipient. The phrases “pharmaceutical or pharmacologically acceptable” refers to molecular entities and compositions that do not produce an adverse, allergic or other untoward reaction when administered to an animal, such as, for example, a human, as appropriate. The preparation of an pharmaceutical composition that contains at least one p21^{Cip1/WAF1} mutant form or additional active ingredient will be known to those of skill in the art in light of the present disclosure, as exemplified by Remington's Pharmaceutical Sciences, 18th Ed. Mack Printing Company, 1990, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the therapeutic or pharmaceutical compositions is contemplated.

The p21^{Cip1/WAF1} mutant form may comprise different types of carriers depending on whether it is to be administered in solid, liquid or aerosol form, and whether it need to be sterile for such routes of administration as injection. The present invention can be administered intravenously, intradermally, intraarterially, intraperitoneally, intralesionally, intracranially, intraarticularly, intraprostaticaly, intrapleurally, intratracheally, intranasally, intravitreally, intravaginally, rectally, topically, intratumorally, intramuscularly, intraperitoneally, subcutaneously, intravesicularlly, mucosally, intrapericardially, orally, topically, locally, using aerosol, injection, infusion, continuous infusion, localized perfusion bathing target cells directly, via a catheter, via a lavage, in cremes, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The actual dosage amount of a composition of the present invention administered to an animal patient can be determined by physical and physiological factors such as body weight, severity of condition, the type of disease being treated, previous or concurrent therapeutic interventions, idiopathy of the patient and on the route of administration. The practitioner responsible for administration will, in any event, determine the concentration of active ingredient(s) in a composition and appropriate dose(s) for the individual subject.

In certain embodiments, pharmaceutical compositions may comprise, for example, at least about 0.1% of an active compound. In other embodiments, the an active compound may comprise between about 2% to about 75% of the weight of the unit, or between about 25% to about 60%, for example, and any range derivable therein. In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., can be administered, based on the numbers described above.

In any case, the composition may comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

The p21^{Cip1/WAF1} mutant form may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine.

In embodiments where the composition is in a liquid form, a carrier can be a solvent or dispersion medium comprising but not limited to, water, ethanol, polyol (e.g., glycerol, propylene glycol, liquid polyethylene glycol, etc), lipids (e.g., triglycerides, vegetable oils, liposomes) and combinations thereof. The proper fluidity can be maintained, for example, by the use of a coating, such as lecithin; by the maintenance of the required particle size by dispersion in carriers such as, for example liquid polyol or lipids; by the use of surfactants such as, for example hydroxypropylcellulose; or combinations thereof such methods. In many cases, it will be preferable to include isotonic agents, such as, for example, sugars, sodium chloride or combinations thereof.

In other embodiments, one may use eye drops, nasal solutions or sprays, aerosols or inhalants in the present invention. Such compositions are generally designed to be compatible with the target tissue type. In a non-limiting example, nasal solutions are usually aqueous solutions designed to be administered to the nasal passages in drops or sprays. Nasal solutions are prepared so that they are similar in many respects to nasal secretions, so that normal ciliary action is maintained. Thus, in preferred embodiments the aqueous nasal solutions usually are isotonic or slightly buffered to maintain a pH of about 5.5 to about 6.5. In addition, antimicrobial preservatives, similar to those used in ophthalmic preparations, drugs, or appropriate drug stabilizers, if required, may be included in the formulation. For example, various commercial nasal preparations are known and include drugs such as antibiotics or antihistamines.

In certain embodiments the p21^{Cip1/WAF1} mutant form is prepared for administration by such routes as oral ingestion. In these embodiments, the solid composition may comprise, for example, solutions, suspensions, emulsions, tablets, pills, capsules (e.g., hard or soft shelled gelatin capsules), sustained release formulations, buccal compositions, troches, elixirs, suspensions, syrups, wafers, or combinations thereof. Oral compositions may be incorporated directly with the food of the diet. Preferred carriers for oral administration comprise inert diluents, assimilable edible carriers or combinations thereof. In other aspects of the invention, the oral composition may be prepared as a syrup or elixir. A syrup or elixir, and may comprise, for example, at least one active agent, a sweetening agent, a preservative, a flavoring agent, a dye, a preservative, or combinations thereof.

In certain preferred embodiments an oral composition may comprise one or more binders, excipients, disintegration agents, lubricants, flavoring agents, and combinations thereof. In certain embodiments, a composition may comprise one or more of the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc.; or combinations thereof the foregoing. When the dosage unit form is a capsule, it may contain, in addition to materials of the above type, carriers such as a liquid carrier. Various other materials may be present as coatings or to otherwise modify the physical form of the dosage unit. For instance, tablets, pills, or capsules may be coated with shellac, sugar or both.

Additional formulations which are suitable for other modes of administration include suppositories. Suppositories are solid dosage forms of various weights and shapes, usually medicated, for insertion into the rectum, vagina or urethra. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10%, and preferably about 1% to about 2%.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and/or the other ingredients. In the case of sterile powders for the preparation of sterile injectable solutions, suspensions or emulsion, the preferred methods of preparation are vacuum-drying or freeze-drying techniques which yield a powder of the active ingredient plus any additional desired ingredient from a previously sterile-filtered liquid medium thereof. The liquid medium should be suitably buffered if necessary and the liquid diluent first rendered isotonic prior to injection with sufficient saline or glucose. The preparation of highly concentrated compositions for direct injection is also contemplated, where the use of DMSO as solvent is envisioned to result in extremely rapid penetration, delivering high concentrations of the active agents to a small area.

The composition must be stable under the conditions of manufacture and storage, and preserved against the contaminating action of microorganisms, such as bacteria and fungi. It will be appreciated that endotoxin contamination should be kept minimally at a safe level, for example, less that 0.5 ng/mg protein.

In particular embodiments, prolonged absorption of an injectable composition can be brought about by the use in the compositions of agents delaying absorption, such as, for example, aluminum monostearate, gelatin or combinations thereof.

XII. Site-Directed Mutagenesis

Structure-guided site-specific mutagenesis represents a powerful tool for the dissection and engineering of protein-ligand interactions (Wells, 1996, Braisted et al, 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation- bearing strand. Thus, a heteroduplex is formed, wherein one strand encodes the original non- mutated sequence, and the second strand bears the desired mutation. This heteroduplex vector is then used to transform appropriate host cells, such as E. coli cells, and clones are selected that include recombinant vectors bearing the mutated sequence arrangement.

Comprehensive information on the functional significance and information content of a given residue of protein can best be obtained by saturation mutagenesis in which all 19 amino acid substitutions are examined. The shortcoming of this approach is that the logistics of multiresidue saturation mutagenesis are daunting (Warren et al., 1996, Brown et al., 1996; Zeng et al., 1996; Burton and Barbas, 1994; Yelton et al., 1995; Jackson et al, 1995; Short et al., 1995; Wong et al., 1996; Hilton et al., 1996). Hundreds, and possibly even thousands, of site specific mutants must be studied. However, improved techniques make production and rapid screening of mutants much more straightforward. See also, U.S. Pat. Nos. 5,798,208 and 5,830,650, for a description of “walk-through” mutagenesis.

Other methods of site-directed mutagenesis are disclosed in U.S. Pat. Nos. 5,220,007; 5,284,760; 5,354,670; 5,366,878; 5,389,514; 5,635,377; and 5,789,166.

### EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Methods and Materials

The PI-3K inhibitor wortmannin and the DNA dye Dapi were obtained from Roche Molecular Biochemicals. The anti-flag and anti-HA (12C5) antibodies were obtained from Sigma Chemical Co. (St. Louis, Mo.) and Roche Molecular Biochemicals (Nutley, N.J.), respectively. The Akt and p21^{Cip1/WAF1} antibodies were obtained from New England Biolabs (Beverly, Mass.) and Santa Cruz Biotech (Santa Cruz, Calif.), respectively.

Constructs of p21^{Cip1/WAF1}

A BamH I site and an EcoR I site were generated near the start and termination codons, respectivly, in human wild-type p21^{Cip1/WAF1} by PCR and subcloned into the expression vector pcDNA3. Site-directed mutagenesis was performed according to the manufacturer's protocol (Clontech Inc.; La Jolla, Calif.). Threonine 145 in p21^{Cip1/WAF1} was replaced by either Ala or Asp by using the following primers: for T145A, 5′-CGAAAACGGCGGCAGGCCAGCATGAC-3′ (SEQ ID NO:74), and for T145D, 5′-CGAAAACGGCGG-CAGGACAGCATGAC -3′ (SEQ ID NO:75). For the colony-formation assay in DN-Akt 3T3 cells, a BamHI and EcoRI fragment containing wild-type or mutant p21^{Cip1/WAF1} was subcloned into the expression vector pcDNA3-hygromycin. The sequences of the wild-type and mutant p21^{Cip1/WAF1} constructs were verified by automated sequencing. To generate a wild-type and mutant p21^{Cip1/WAF1} GST-tagged bacterial expression constructs, the same fragments containing the wild-type and mutant p21^{Cip1/WAF1} were subcloned into the bacterial expression vector pGEX4T-3 (Pharmacia Biotech; Piscataway, N.J.). Wild-type and mutant p21^{Cip1/WAF1} proteins were inducible expressed in E.coli strain BL21 and purified by glutathione sepharose chromatography (Pharmacia Biotech; Piscataway, N.J.).

Cell Culture

NIH3T3, HER-2/neu 3T3, breast cancer cell MDA-MB453, p21^{Cip1/WAF1}-deficient MEF, and 293T cells were culture in DMEM/F12 supplemented with 10% fetal bovine serum. The DN-Akt transfectants of HER-2/neu 3T3 and MDA-MB453 cells were grown under the same conditions except that 450 μg/ml G418 was added to the culture medium (Zhou et al., 2000). The 293T cells were transfected by the calcium phosphate technique and p21^{Cip1/WAF1}-deficient cells by the liposome method.

In vitro Growth Rate Analysis

The in vitro growth rates of the cell lines were assessed by counting the cells with a Coulter counter or by the MTT assay as described previously (Van der Geer and Hunter, 1994).

[³H]Thymidine Incorporation Assay

The cell proliferation rates of the cell lines were analyzed by measuring [³H]thymidine incorporation as described previously (Van der Geer and Hunter, 1994).

Endoproteinase Cleavage and Two-dimensional Phosphopeptide Analysis

Two-dimensional analysis of p21^{Cip1/WAF1} phosphopeptides was performed with a HTLE-7000 electrophoresis system from CBS Scientific (Del Mar, CA) as described previously (Gatti and Traugh, 1999). Briefly, MDA-MB453 cells were incubated with 1.5 mCi/ml [³²P]orthophosphate for 3.5 hours followed by 30 min of stimulation with insulin with or without LY294002. Endogenous p21^{Cip1/WAF1} was immunoprecipitated, blotted, and visualized by autoradiography. The ³²P-labelled p21^{Cip1/WAF1} protein on the nitrocellulose membrane was excised and digested with TPCK-trypsin (Sigma Chemical Co; St. Louis, Mo.). The completely digested phosphopeptides were spotted on 20×20 cm thin-layer cellulose plates and separated in the first dimension by electrophoresis at 1000 V for 35 min in pH 1.9 buffer (2.2% formic acid and 7.8% acetic acid). The cellulose plates were then placed in a chromatography tank containing phosphochromatography buffer (38% n-butanol, 25% pyridine, and 7.5% acetic acid) for 6-8 h to separate the phosphopeptides in the second dimension. The dried cellulose plates were then exposed to Kodak X-AR film.

Edman Degradation

Modified manual Edman degradation was performed as described previously (Nabel et al., 1998). Briefly, phosphopeptides were covalently coupled to Sequelon-AA discs (Perseptive Biosystem Inc.) and subjected to consecutive cycles of Edman degradation. After each cycle, the discs were treated with trifluoroacetic acid to cleave and release the N-terminal amino acid, and the radioisotope released was measured by Cerenkov counting.

In Vitro Kinase Assay

293T cells (0.2×10⁶) were transfected with 20 μg of HA-tagged CA-Akt or DN-Akt. After 48 hr of transfection, Akt was immunoprecipitated from cell extracts and incubated with 5 μg of purified GST-p21^{Cip1/WAF1} (wild-type or mutant) in the presence of 5 μCi of [γ-32P]ATP and 50 mM cold ATP in a kinase buffer for 30 min at 30° C. The reaction products were resolved by SDS-PAGE, and the ³²P-labeled proteins were visualized by autoradiography.

³²P-orthophosphate Labeling

293T cells (0.2×10⁶) were cotransfected with 18 μg of CA-Akt or DN-Akt and 2 μg of wild-type or mutant p21^{Cip1/WAF1}. After 36 hr of transfection, the cells were starved for 12 hr and then incubated with phosphate-free medium for 1 hr. The cells were then labeled with 1 mCi/ml ³²P-orthophosphate for 3 hr. The cells were then lysed, and p21^{Cip1/WAF1} was immunoprecipitated from the cell extracts and separated by 12% SDS-PAGE. The incorporation of ³²P-phosphate was measured by autoradiography.

Immunoprecipitation and Immunoblotting

Cells were washed twice with PBS and scraped into 500 ml of lysis buffer. After a brief sonication, the lysate was centrifuged at 14,000× g for 10 min at 4° C. to remove the insoluble cell debris. Immunoprecipitation and immunoblotting were performed as described previously (Zhou et al., 2000).

In Situ Immunofluorescent Staining

Approximately 0.2×10⁶ p2^{-/-} MEF cells were plated in 100-mm plates and cotransfected with 9 μg of CA-Akt or DN-Akt and 1 μg of wild-type or mutant p21^{Cip1/WAF1} by using liposome. After 36 hr of incubation, the cells were trypsinized and plated into chamber slides for another 12 hr. After fixation of the samples in cold acetone for 10 minutes at 4° C., the cellular localization of p21^{Cip1/WAF1} was determined by using a monoclonal antibody against human p21^{Cip1/WAF1} (Santa Cruz Biotech; Santa Cruz, Calif.) diluted 1:100. After extensive washing in phosphate-buffered saline, the samples were further incubated with Texas Red-conjugated goat anti-mouse IgG (diluted 1:400) plus dapi (0.1 μg/ml) for 1 hr. After extensive washing, the samples were examined under a fluorescent microscope (Carl Zeiss, Inc.; Thormwood, N.Y.). The nonspecific reaction of secondary antibody was ruled out by the absence of fluorescence under the microscope.

Cellular Fractionation

Approximate 2×10⁷ cells were pelleted and resuspended in 800 μl of buffer A (10 mM HEPES, pH 7.4, 1 mM EDTA, and 1 mM DTT) containing the protease inhibitors PMSF, leupeptin, aprotinin and pepstain. After incubation on ice for 10 min, the cells were homogenized with 10 strokes in a Dounce homogenizer. Then, the cells were examined under the microscope to confirm that more than 98% of the cells were lysed. After a brief centrifugation at 4° C., the supernatant (cytoplasmic fraction) was collected and the pellet was washed twice with 400 μl of buffer B, and then resuspended in 150 ml of buffer C with gentle rocketing for 30 min at 4° C. (Asada et al., 1999). After centrifugation, the supernatant (nuclear fraction) was collected. The amount of protein in the cytoplasmic and the nuclear fractions were determined with a protein assay kit (Bio-rad) and the protein was subjected to immunoblotting.

Colony-Formation Assay

The colony-formation assay was used to measure the inhibition activity of p21^{Cip1/WAF1} and its mutant. Wild-type or mutant p21^{Cip1/WAF1} (Ti45A or T145D) or the vector pcDNA3 (2 μg of each) was transfected into NIH3T3, HER-2/neu 3T3, and DN-Akt 3T3 cells in six-well plates by using liposome. After 48 hr of transfection, the cells were trypsinized and evenly distributed into four 100-mm culture plates. The cells were selected with 700 μg/ml G418 (or 100 μg/ml hygromycin for DN-Akt 3T3 cells, as they contain the neomycin-resistance gene) for 3 weeks.

Bromodeoxyuridine (BrdU) Incorporation

The Brdu incorporation assay was also used to measure the inhibition activity of p21^{Cip1/WAF1} and its mutant. Vector containing membrane-bound green fluorescence protein (GFP) (1 μg) and wild-type or mutant p21^{Cip1/WAF1} (T145A or T145D) or the vector pcDNA3 (9 μg of each) were cotransfected into NIH3T3, HER-2/neu 3T3, and DN-Akt 3T3 cells by using liposome. After 48 hr of incubation, the cells were labeled with Brdu for 1 hr and then fixed with 70% ethanol. The cells were then stained with anti-Brdu antibody and incubated with fluorescent-conjugated secondary antibody. After extensive washing, the cells were sorted for GFP, and the incorporation of Brdu was measured by using FACS analysis.

### Example 2

### The Akt Pathway is Required for HER-2/neu-Mediated Cell Proliferation

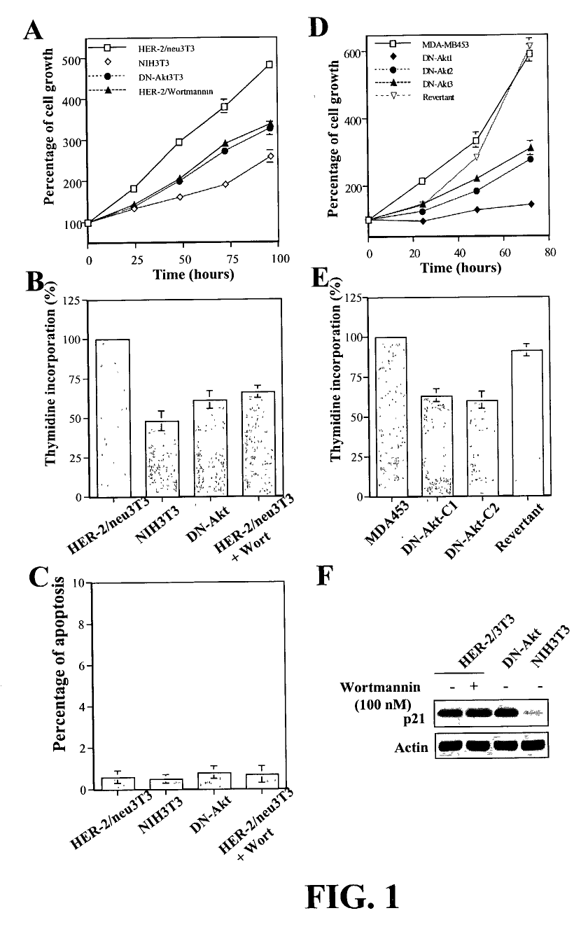

To study the effect of Akt on HER-2/neu-mediated cell proliferation, a model system was used that consists of NIH3T3 cells, HER-2/neu 3T3 cells (HER-2/neu transformed NIH3T3 cells), and DN-Akt 3T3 cells (HER-2/neu 3T3 cells transfected with DN-Akt, a kinase-dead mutant of Akt) (Zhou et al., 2000). As expected, the HER-2/neu 3T3 cells grew much faster than did the parental NIH3T3 cells (FIG. 1A). The Akt pathway is known to be constitutively activated in HER-2/neu 3T3 cells (Zhou et al., 2000), and when this pathway was blocked by DN-Akt in DN-Akt 3T3 cells, cell growth became slower (FIG. 1A). This was not due to the heterogeneity of the cell clones, because a specific PI-3K inhibitor, wortmannin, also produced a similar slowing of growth in HER-2/neu 3T3 cells (FIG. 1A). When the DNA synthesis rate was determined by measuring [³H]thymidine incorporation, HER-2/neu 3T3 cells also had greater DNA synthesis than did the parental NIH3T3 cells (FIG. 1B). HER-2/neu-induced DNA synthesis was significantly inhibited by blocking Akt pathway with either wortmannin or DN-Akt. As the net cell growth rate depends on a fine balance between the cell proliferation rate and the cell death rate, it was also examined whether apoptosis contributes to the difference in growth in these cells. There was no significant difference in apoptosis among these cells as measured by FACS analysis (FIG. 1C). Therefore, the reduction in cell growth in DN-Akt 3T3 cells was most likely due to the decrease in cell proliferation. To extend these findings, the same experiments were performed in another HER-2/neu-overexpressing breast cancer cell line, MDA-MB453, and stable DN-Akt transfectants of it (Zhou et al., 2000). Like the HER-2/neu 3T3 and DN-Akt 3T3 cell lines, three independent clones of DN-Akt transfectants showed reductions in cell growth and DNA synthesis (FIG. 1D and E), but no difference was observed in apoptosis assayed by FACS analysis. A revertant that had lost DN-Akt during culture had cell growth and DNA synthesis rates almost identical to those of parental MDA-MB453 cells (FIG. 1D and E). Taken together, these results indicated that the Akt pathway was required for HER-2/neu-mediated cell proliferation and that inhibition of the Akt pathway by either a PI-3K inhibitor or DN-Akt significantly reduced cell proliferation.

Cell proliferation is tightly regulated by the family of cyclin-dependent kinase (CDK) inhibitors. p21^{Cip1/WAF1} was shown to play a critical role in regulating cell proliferation (Harper et al., 1993; El-Deiry et al., 1993; Noda et al., 1994). Therefore, the expression of p21^{Cip1/WAF1} was measured in these cells. The expression of p21^{Cip1/WAF1} was significantly higher in HER-2/neu 3T3 cells than in the parental cells (FIG. 1F), consistent to the previous report (Yu et al., 1998). Inhibition of the Akt pathway by DN-Akt in DN-Akt 3T3 cells did not significantly change p21^{Cip1/WAF1} expression (FIG. 1F). This result could not account for the HER-21/neu-induced cell proliferation and suggested that there may be an unknown mechanism that overrides the enhanced expression of p21^{Cip1/WAF1} in HER-2neu 3T3 cells.

### Example 3

### p21^{Cip1/WAF1} Threonine 145 Phosphorylation In Vivo is Induced by Insulin and Inhibited by PI-3K Inhibitor

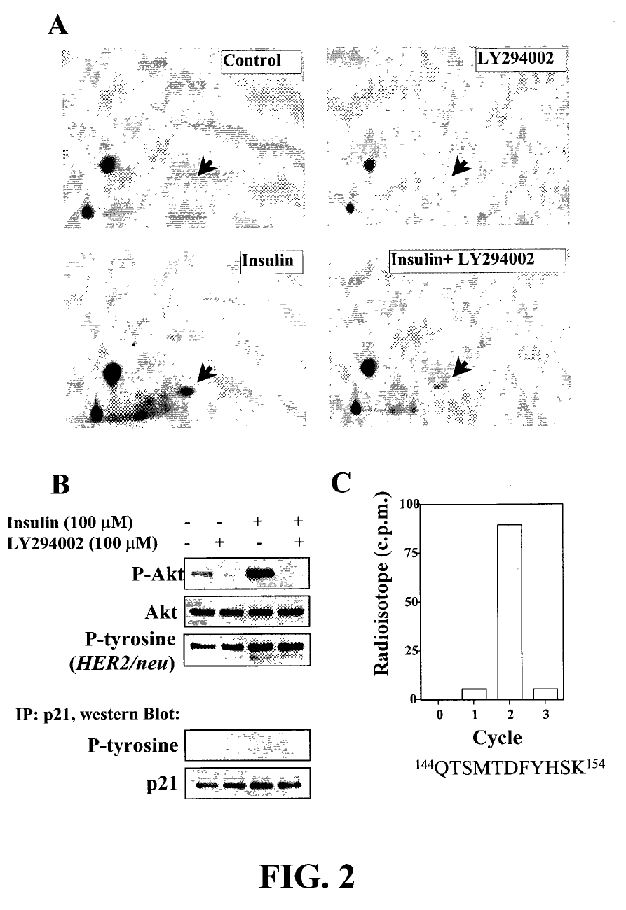

Because protein phosphorylation plays a major role in the regulation of protein function and p21^{Cip1/WAF1} was shown to be phosphorylated in vitro by PKC (Scott et al., 2000), the phosphorylation pattern of endogenous p21^{Cip1/WAF1} was examined by using in vivo [³²P]orthophosphate labeling and two-dimensional phosphopeptide analysis (FIG. 2A). A strong phosphorylated peptide spot was observed after induction by insulin, which is known to activate PI-3K/Akt, and the spot was much weaker when the cells were incubated with the PI-3K inhibitor LY294002. This insulin-induced phosphorylation spot was most likely a result of phosphorylation of serine or threonine instead of tyrosine residues in p21^{Cip1/WAF1}, because no tyrosine phosphorylation was detected when endogenous p21^{Cip1/WAF1} was immunoprecipitated and examined by western blotting with antibody specific against tyrosine phosphorylation (FIG. 2B). That no tyrosine phosphorylated endogenous p21^{Cip1/WAF1} was detected was not due to the quality of the antibody, because the same antibody was able to detect tyrosine phosphorylation of HER-2/neu in the same cell lysate (FIG. 2B). The insulin-induced phosphorylation site in p21^{Cip1/WAF1} was further analyzed by in vivo [³²P]orthophosphate labeling, and the trypsin-digested phosphopeptides were then subjected to amino acid sequencing by Edman degradation. The activity of radioisotope was released after the second cycle of Edman degradation (FIG. 2C), but was greatly reduced when the cells were pretreated with PI-3K inhibitor LY294002. Judging from the amino acid sequence of p21^{Cip1/WAF1}, the only trypsin-digested peptide that contains threonine or serine at the second amino acid residue from the N-terminus is, 144-QTSMTDFYHSK-154 (SEQ ID NO:76). Thus, the results indicated that threonine 145 of p21^{Cip1/WAF1} was phosphorylated in vivo and that this phosphorylation could be induced by insulin and inhibited by PI-3K inhibitor.

### Example 4

### Akt Associates with p21^{Cip1/WAF1}

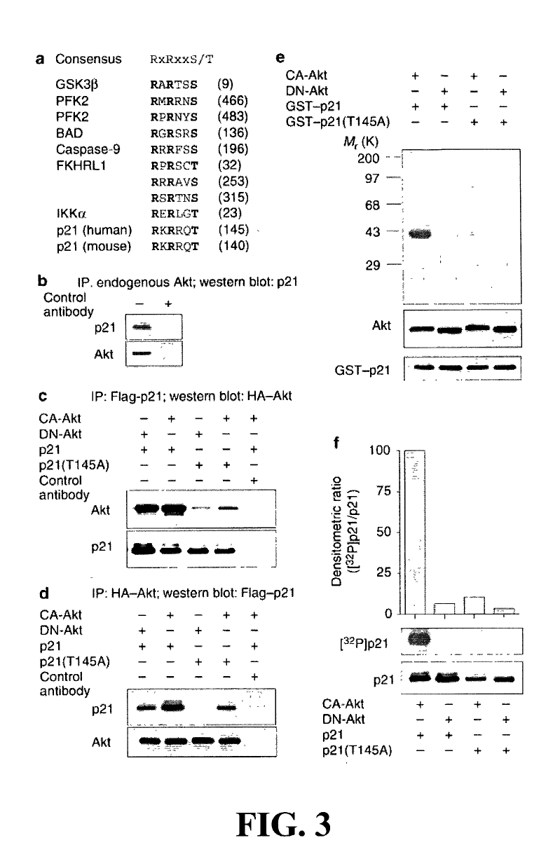

The nuclear localization of p21^{Cip1/WAF1} appears to be responsible for its cell-growth inhibition and is controlled by the NLS at the C-terminus of the molecule (Chen et al., 1995). There is a putative Akt phosphorylation motif in the NLS of p21^{Cip1/WAF1} and this Akt phosphorylation motif was highly conserved among different species (FIG. 3A). The phosphorylated T145 residue of p21^{Cip1/WAF1} detected in FIG. 2 was located in this motif. To test whether Akt interacts with and phosphorylates p21^{Cip1/WAF1} and so regulates its cellular localization, it was first investigated whether Akt associates with p21^{Cip1/WAF1} by co-immunoprecipitation experiments. After immunoprecipitating endogenous Akt from MDA-MB453 cells, the presence of endogenous p21^{Cip1/WAF1} was detected, although while using nonspecific antibody, the presence of endogenous p21^{Cip1/WAF1} could not be detected (FIG. 3B). Also a constitutively active Akt (CA-Akt) or DN-Akt with wild-type or mutant p21^{Cip1/WAF1} (T145A) was cotransfected into 293T cells. After immunoprecipitating the p21^{Cip1/WAF1}, Akt was detected and vice versa (FIG. 3C and D), which suggests that these two molecules are associated. The association was dependent on the kinase status of Akt and the status of p21^{Cip1/WAF1}. p21^{Cip1/WAF1} associated more strongly with CA-Akt than with DN-Akt, and un-phosphorylated p21^{Cip1/WAF1} (T145A) associated much more weakly with Akt than did wild-type p21^{Cip1/WAF1} Furthermore, Akt could phosphorylate p21^{Cip1/WAF1} both in vitro and in vivo, whereas DN-Akt could not (FIG. 3E and F). That no phosphorylation was observed on the mutant p21^{Cip1/WAF1} (T145A), in which threonine 145 in the putative Akt phosphorylation site was replaced with alanine, indicated that Akt interacts with p21^{Cip1/WAF1} and phosphorylates it at threonine 145. This is further supported by the data presented in FIG. 2 that phosphorylation of threonine 145 is induced by insulin and inhibited by LY294002, since Akt is known to be activated by insulin and inhibited by LY294002.

### Example 5

### Threonine 145 of p21^{Cip1/WAF1} is Critical in Determining the Cellular Localization of p21^{Cip1/WAF}1

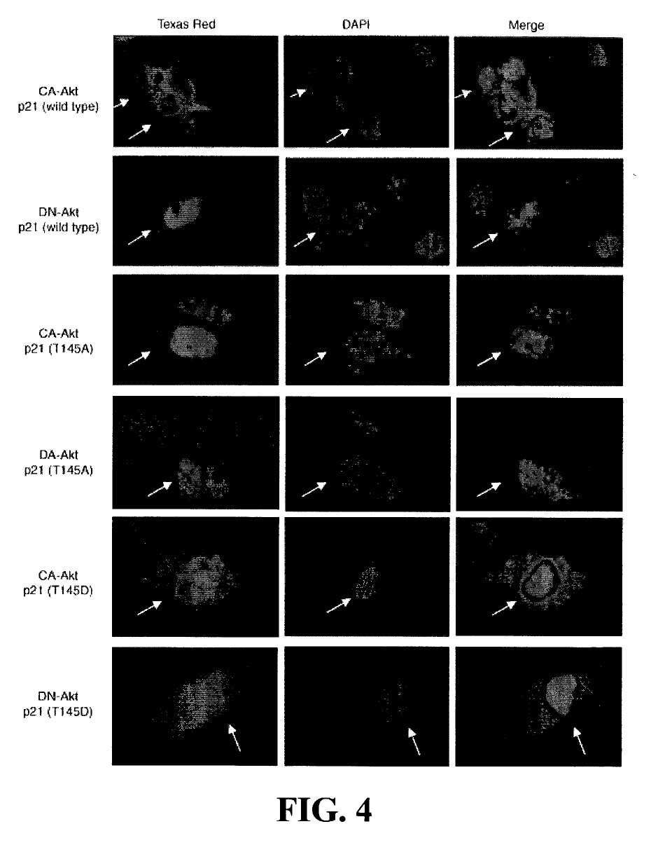

It was next tested whether the activation of Akt affects the cellular localization of p21^{Cip1/WAF1}, as Akt could phosphorylate a critical threonine residue in the NLS of p21^{Cip1/WAF1}. CA-Akt or DN-Akt were cotransfected with wild-type or mutant p21^{Cip1/WAF1} (T145A and T145D) into p21^{-/-} MEF cells, and the cellular localization of p21^{Cip1/WAF1} was examined by immunofluorescence analysis. As seen in FIG. 4, wild-type p21^{Cip1/WAF1} was found predominantly in the cytoplasm in the presence of CA-Akt but was predominantly in the nucleus when DN-Akt was introduced. Mutation in the critical threonine residue in T145A abolished cytoplasmic localization even in the presence of CA-Akt. Mutant p21^{Cip1/WAF1} (T145D), in which threonine 145 was replaced with aspartate acid to mimic the phosphorylation of p21^{Cip1/WAF1} by Akt, was found predominantly in the cytoplasm no matter whether the Akt pathway was turned on by CA-Akt or shut off by DN- Akt. These results strongly indicated that threonine 145 of p21^{Cip1/WAF1} is critical in determining the cellular localization of p21^{Cip1/WAF1} and phosphorylation of threonine 145 of p21^{Cip1/WAF1} by Akt resulted in the cytoplasmic localization of p21^{Cip1/WAF1}. Taken together, the results indicated that activated Akt could interact with p21^{Cip1/WAF1} and phosphorylated the threonine 145 residue in the NLS of p21^{Cip1/WAF1}, which led to cytoplasmic localization of p21^{Cip1/WAF1}.

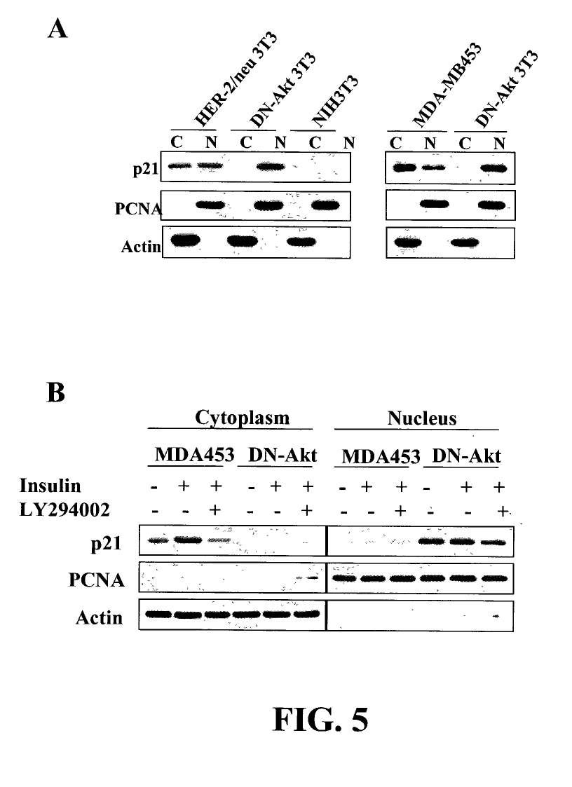

The above experiments were carried out in cells that were transiently transfected with exogenous gene. To examine whether endogenous p21^{Cip1/WAF1} can be regulated in a similar way, biochemical cellular fractionation was first performed to determine the localization of endogenous p21^{Cip1/WAF1} in HER-2/neu-overexpressing cells and their DN-Akt transfectants. It was found that p21^{Cip1/WAF1} was predominantly located in the cytoplasm in both HER-2/neu 3T3 cells and MDA-MB453 cells, in which Akt was constitutively activated (FIG. 5A). However, when the Akt pathway was blocked by DN-Akt, p21^{Cip1/WAF1} was found primarily in the nucleus in both DN-Akt 3T3 and DN-Akt/MB453 cells (FIG. 5A). As a control, actin and proliferating cell nuclear antigen (PCNA) were used as cytoplasmic and nuclear markers, respectively, to confirm that the cellular localization of p21^{Cip1/WAF1} was not due to contamination. Similar results were obtained when HER-2/neu 3T3 and its DN-Akt transfectants were stained for endogenous p p21^{Cip1/WAF1} using the same techniques as described in FIG. 4. To further investigate whether the phosphorylation of endogenous p21^{Cip1/WAF1} by Akt affects its subcellular localization, MDA-MB453 cells were treated with insulin, which is known to be a potent Akt activator (FIG. 2B). It was found that endogenous p21^{Cip1/WAF1} was present in both the nucleus and cytoplasm of MDA-MB453 cells, and the cytoplasmic fraction of p21^{Cip1/WAF1} was significantly increased upon stimulation of insulin (FIG. 5B). It should be noted that equal amount of proteins were loaded on the gel, however, the protein content in cytoplasm and nucleus was about 10 to 1 in the cellular fraction. In contrast to the parental cells, in DN-Akt transfectants endogenous p21^{Cip1/WAF1} was mainly in the nucleus, and the nucleus localization was not affected by insulin treatment. Taken together, these results indicate that the distribution of endogenous p21^{Cip1/WAF1} can be regulated by extracellular stimuli, such as insulin, through the PI-3K/Akt pathway.

### Example 6

### Overexpression OF HER-21/neu Can Regulate the Cellular Distribution of p21^{Cip1/WAF1} by the Activation of Akt in Tumor Tissue

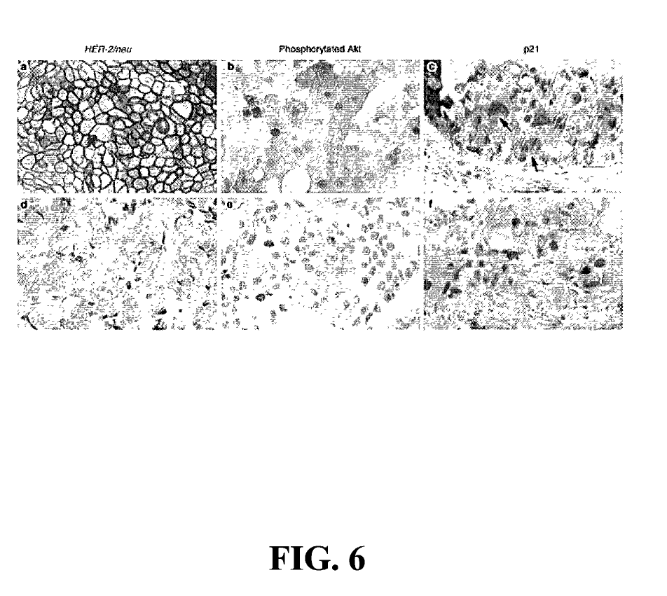

These results clearly established the regulation of p21^{Cip1/WAF1} by the HER-2/neu-Akt pathway in cell culture. To examine whether this phenomenon also existed in tumor tissues, the levels of activated (phosphorylated) Akt and the cellular localization of p21^{Cip1/WAF1} in 5 HER-2/neu-positive and 5 HER-2/neu-negative human breast tumors were compared by immunostaining with antibodies specific to phosphorylated Akt and specific to p21^{Cip1/WAF1}. Consistant with the previous study (Zhou et al., 2000), Akt was activated in all of 5 HER-2/neu-positive breast tumor tissues. In these 5 breast tumor tissues, it was found that p21^{Cip1/WAF1} was present in both the nucleus and the cytoplasm. In contrast, in all 5 HER-2/neu-negative breast tumor tissues examined, Akt was not activated, and p21^{Cip1/WAF1} was primarily localized in the nucleus. A representative experiment is shown in FIG. 6. The tumor staining data support the observation in cell culture and further strengthen the evidence that overexpression of HER-2/neu can regulate the cellular distribution of p21^{Cip1/WAF1} by the activation of Akt.

### Example 7

### T145A and T145D Mutants of p21^{Cip1/WAF1} Have Different Subcellular Localizations and Opposite Growth Inhibition Activities

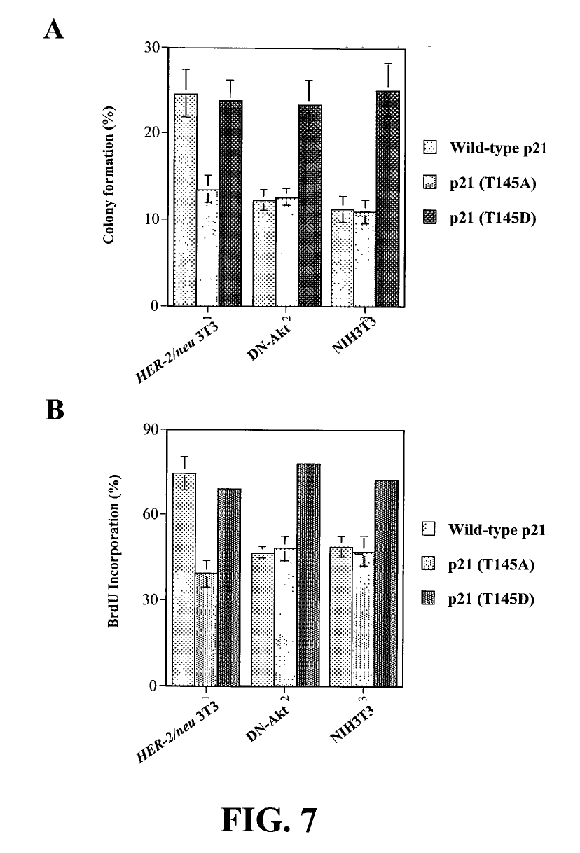

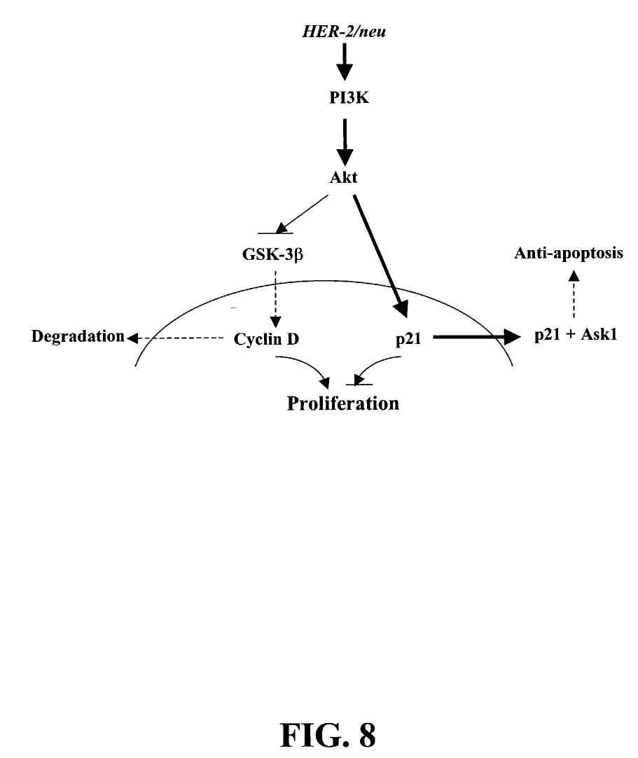

Because Akt could phosphorylate p21^{Cip1/WAF1} and caused its cytoplasmic localization, it was next addressed whether the phosphorylation status of p21^{Cip1/WAF1} at threonine 145 affected the cell growth inhibitory activity of p21^{Cip1/WAF1} Wild-type p21^{Cip1/WAF1} and its mutants T145A (which could not be phosphorylated) and T145D (in which threonine 145 was mutated to aspartic acid to mimic the phosphorylation) were transfected into NIH3T3, HER-2/neu 3T3, and DN-Akt 3T3 cells, and their growth inhibitory activity was measured by using the colony-formation assay. As seen in FIG. 7A, wild-type p21^{Cip1/WAF1} did not effectively inhibit the growth of HER-2/neu 3T3 cells, in which Akt is constitutively activated, compared with NIH3T3 and DN-Akt 3T3 cells. However, T145A, which had lost the Akt phosphorylation site, had similar growth-inhibition activities in these three cell lines, and the activities were independent of the activation of Akt. In contrast, T145D, which mimicked the phosphorylation of p21^{Cip1/WAF1}, behaves similar to wild-type p21^{Cip1/WAF1} in HER-2/neu 3T3 cells in all three cell lines. When DNA synthesis rate was measured by bromodeoxyuridine (BrdU) incorporation between wild-type and mutant p21^{Cip1/WAF1} (FIG. 7B), similar to the results of colony-formation assay, the growth inhibition activity of wild-type p21^{Cip1/WAF1} was regulated by the Akt status. T145A exhibited its inhibition activity independent of Akt status. T145D is also independent of Akt status and its suppression effect is weaker in all three cells and is comparable to wild-type p21^{Cip1/WAF1} in HER-2/neu 3T3 cells, in which p21^{Cip1/WAF1} is predominantly located in the cytoplasm. How T145D only partially lost its ability to inhibit cell growth is not clear. One explanation is that T145D may still retain its ability to inhibit the function of cdk2, cdk4, and cyclin D, which are shuttle molecules between nucleus and cytoplasm. Alternatively, cytoplasmic p21^{Cip1/WAF1} may also have effects on cell growth. Further systemic study is required to elucidate the detailed mechanism. Taken together, these results indicate that phosphorylation of threonine 145 in the NLS of p21^{Cip1/WAF1} by Akt triggers cellular localization and then regulate the growth-inhibitory activity of p21^{Cip1/WAF1}.

### Example 8

### p21^{Cip1/WAF1} T145A and/or T145D Mutants As Therapeutic Agents

The p21-T145A mutant as it relates to its anti-tumor activity is tested in an animal study. The p21-T145A mutant is delivered by a vector, such as a liposome or adenoviral vector, into nude mice models for its anti-tumor activity. Once the anti-tumor activity is demonstrated, potential toxicity is further examined using immunocompetent mice, followed by clinical trials.

In a specific embodiment, the preferential growth inhibitory activity of mutant p21(T145A) is tested in animal. Briefly, HER-2/neu overexpressing breast cancer cell lines (such as SKBR3 and MDA-MB361) are administered into mammary fat-pad of nude mice to generate a breast xcnografted model. After the tumors reach a particular size, the p21 mutant or wild-type p21 control is intravenously injected into the mouse in an admixture with an acceptable carrier, such as liposomes. The tumor sizes and survival curve from these treatments are compared and statistically analyzed. In a preferred embodiment, the mutant p21 (Ti45A) is better and preferentially inhibits the growth of tumor compared to that of wild-type p21.

### Example 9

### Clinical Trials

This example is concerned with the development of human treatment protocols using the p21^{Cip1/WAF1} mutant protein, peptide, or polypeptide or a nucleic acid encoding the p21^{Cip1/WAF1} mutant protein, peptide, or polypeptides, alone or in combination with other anti-cancer drugs. The p21^{Cip1/WAF1} mutant protein, peptide, or polypeptide or a nucleic acid encoding the p21^{Cip1/WAF1} mutant protein, peptide, or polypeptides, and anti-cancer drug treatment will be of use in the clinical treatment of various cancers involving, for example, Akt activation in which transformed or cancerous cells play a role. Such treatment will be particularly useful tools in anti-tumor therapy, for example, in treating patients with ovarian, breast, prostate, pancreatic, brain, colon, and lung cancers that are resistant to conventional chemotherapeutic regimens.

The various elements of conducting a clinical trial, including patient treatment and monitoring, will be known to those of skill in the art in light of the present disclosure. The following information is being presented as a general guideline for use in establishing the p21^{Cip1/WAF1} mutant protein, peptide, or polypeptide or a nucleic acid encoding the p21^{Cip1/WAF1} mutant protein, peptide, or polypeptides, in clinical trials.

Patients with advanced, metastatic breast, epithelial ovarian carcinoma, pancreatic, colon, or other cancers chosen for clinical study will typically be at high risk for developing the cancer, will have been treated previously for the cancer which is presently in remission, or will have failed to respond to at least one course of conventional therapy. In an exemplary clinical protocol, patients may undergo placement of a Tenckhoff catheter, or other suitable device, in the pleural or peritoneal cavity and undergo serial sampling of pleural/peritoneal effusion. Typically, one will wish to determine the absence of known loculation of the pleural or peritoneal cavity, creatinine levels that are below 2 mg/dl, and bilirubin levels that are below 2 mg/dl. The patient should exhibit a normal coagulation profile.

In regard to the the p21^{Cip1/WAF1} mutant protein, peptide, or polypeptide or a nucleic acid encoding the p21^{Cip1/WAF1} mutant protein, peptide, or polypeptides, and other anti-cancer drug administration, a Tenckhoff catheter, or alternative device may be placed in the pleural cavity or in the peritoneal cavity, unless such a device is already in place from prior surgery. A sample of pleural or peritoneal fluid can be obtained, so that baseline cellularity, cytology, LDH, and appropriate markers in the fluid (CEA, CA15-3, CA 125, PSA, p38 (phosphorylated and un-phosphorylated forms), Akt (phosphorylated and un-phosphorylated forms) and in the cells (p21^{Cip1/WAF1} mutant proteins, peptides or polypeptides or nucleic acids encoding the same) may be assessed and recorded.

In the same procedure, the p21^{Cip1/WAF1} mutant protein, peptide, or polypeptide or a nucleic acid encoding the p21^{Cip1/WAF1} mutant protein, peptide, or polypeptides, may be administered alone or in combination with the other anti-cancer drug. The administration may be in the pleural/peritoneal cavity, directly into the tumor, or in a systemic manner. The starting dose may be 0.5 mg/kg body weight. Three patients may be treated at each dose level in the absence of grade>3 toxicity. Dose escalation may be done by 100% increments (0.5 mg, 1 mg, 2 mg, 4 mg) until drug related grade 2 toxicity is detected. Thereafter dose escalation may proceed by 25% increments. The administered dose may be fractionated equally into two infusions, separated by six hours if the combined endotoxin levels determined for the lot of the E1A protein, peptide, or polypeptide or a nucleic acid encoding the p21^{Cip1/WAF1} mutant protein, peptide, or polypeptides, and the lot of anti-cancer drug exceed 5 EU/kg for any given patient.

The the p21^{Cip1/WAF1} mutant protein, peptide, or polypeptide or a nucleic acid encoding the p21^{Cip1/WAF1} mutant protein, peptide, or polypeptides, and/or the other anti-cancer drug combination, may be administered over a short infusion time or at a steady rate of infusion over a 7 to 21 day period. The the p21^{Cip1/WAF1} mutant protein, peptide, or polypeptide or a nucleic acid encoding the p21^{Cip1/WAF1} mutant protein, peptide, or polypeptides, infusion may be administered alone or in combination with the anti-cancer drug and/or emodin like tyrosine kinase inhibitor. The infusion given at any dose level will be dependent upon the toxicity achieved after each. Hence, if Grade II toxicity was reached after any single infusion, or at a particular period of time for a steady rate infusion, further doses should be withheld or the steady rate infusion stopped unless toxicity improved. Increasing doses of the p21^{Cip1/WAF1} mutant protein, peptide, or polypeptide or a nucleic acid encoding the mutant protein, peptide, or polypeptides, in combination with an anti-cancer drug will be administered to groups of patients until approximately 60% of patients show unacceptable Grade III or IV toxicity in any category. Doses that are ⅔ of this value could be defined as the safe dose.

Physical examination, tumor measurements, and laboratory tests should, of course, be performed before treatment and at intervals of about 3-4 weeks later. Laboratory studies should include CBC, differential and platelet count, urinalysis, SMA-12-100 (liver and renal function tests), coagulation profile, and any other appropriate chemistry studies to determine the extent of disease, or determine the cause of existing symptoms. Also appropriate biological markers in serum should be monitored e.g. CEA, CA 15-3, p38 (phosphorylated and non-phopshorylated forms) and Akt (phosphorylated and non-phosphorylated forms), p185, etc.

To monitor disease course and evaluate the anti-tumor responses, it is contemplated that the patients should be examined for appropriate tumor markers every 4 weeks, if initially abnormal, with twice weekly CBC, differential and platelet count for the 4 weeks; then, if no myelosuppression has been observed, weekly. If any patient has prolonged myelosuppression, a bone marrow examination is advised to rule out the possibility of tumor invasion of the marrow as the cause of pancytopenia. Coagulation profile shall be obtained every 4 weeks. An SMA-12-100 shall be performed weekly. Pleural/peritoneal effusion may be sampled 72 hours after the first dose, weekly thereafter for the first two courses, then every 4 weeks until progression or off study. Cellularity, cytology, LDH, and appropriate markers in the fluid (CEA, CA15-3, CA 125, k167 and Tunel assay to measure apoptosis, Akt) and in the cells (Akt) may be assessed. When measurable disease is present, tumor measurements are to be recorded every 4 weeks. Appropriate radiological studies should be repeated every 8 weeks to evaluate tumor response. Spirometry and DLCO may be repeated 4 and 8 weeks after initiation of therapy and at the time study participation ends. An urinalysis may be performed every 4 weeks.

Clinical responses may be defined by acceptable measure. For example, a complete response may be defined by the disappearance of all measurable disease for at least a month. Whereas a partial response may be defined by a 50% or greater reduction of the sum of the products of perpendicular diameters of all evaluable tumor nodules or at least 1 month with no tumor sites showing enlargement. Similarly, a mixed response may be defined by a reduction of the product of perpendicular diameters of all measurable lesions by 50% or greater with progression in one or more sites.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.

### PATENTS

U.S. Pat. No. 4,554,101

U.S. Pat. No. 4,683,202

U.S. Pat. No. 4,797,368

U.S. Pat. No. 5,220,007

U.S. Pat. No. 5,284,760

U.S. Pat. No. 5,354,670

U.S. Pat. No. 5,366,878

U.S. Pat. No. 5,389,514

U.S. Pat. No. 5,635,377

U.S. Pat. No. 5,641,484

U.S. Pat. No. 5,670,488

U.S. Pat. No. 5,789,166

U.S. Pat. No. 5,807,692

U.S. Pat. No. 5,863,904

U.S. Pat. No. 5,871,968

U.S. Pat. No. 5,928,906

U.S. Pat. No. 5,925,565

U.S. Pat. No. 5,928,906

U.S. Pat. No. 5,935,819

U.S. Pat. No. 6,057,300

### PUBLICATIONS

Ahmed, N. N., Gries, H. L., Bellacosa, A., Chan, T. O., & Tsichlis, P. N. (1997) Transduction of interleukin-2 antiapoptotic and proliferative signals via Akt protein kinase. Proc. Natl. Acad. Sci. U.S.A. 94, 3627-3632.

Almendro N, Bellon T, Rius C, Lastres P, Langa C, Corbi A, Bernabeu C. Cloning of the human platelet endothelial cell adhesion molecule-1 promoter and its tissue-specific expression. Structural and functional characterization. J. Immunol. (1996) 157(12):5411-21.

Andjelkovic, M., et al. (1997) Role of translocation in the activation and function of protein kinase B. J. Biol. Chem. 272, 31515-31524.

Arap et al., 1995

Asada, M., et al. (1999) Apoptosis inhibitory activity of cytoplasmic p21Cip1/WAF1 in monocytic differentiation. EMBO. 18, 1223-1234.

Baichwal V R, Sugden B. Latency comes of age for herpesviruses. Cell. (1988) 52(6):787-9.

Bakhshi A, Jensen J P, Goldman P, Wright J J, McBride O W, Epstein A L, Korsmeyer S J. Cloning the chromosomal breakpoint of t(14; 18) human lymphomas: clustering around JH on chromosome 14 and near a transcriptional unit on 18. Cell. (1985) 41(3):899-906.

Benvenisty N, Reshef L. Direct introduction of genes into rats and expression of the genes. Proc Natl Acad Sci U S A. (1986) 83(24):9551-5.

Bishop, 1987

Braisted A C, Wells J A. Minimizing a binding domain from protein A. Proc Natl Acad Sci U S A. (1996) 93(12):5688-92.

Brennan, P., et al. (1997) Phosphatidylinositol 3-kinase couples the interleukin-2 receptor to the cell cycle regulator E2F. Immunity 7, 679-689.

Brown M, Rittenburg M B, Chen C, Roberts V A. Tolerance of single, but not multiple, amino acid replacements in antibody VH CDR 2: a means of minimizing B cell wastage from somatic hypermutation? J. Immunol. (1996) 156(9):3285-91.

Brunet, A., et al. (1999) Akt promotes cell survival by phosphorylating and inhibiting a forkhead transcription factor. Cell 96, 857-868.

Bullrich F, MacLachlan T K, Sang N, Druck T, Veronese M L, Allen S L, Chiorazzi N, Koff A, Heubner K, Croce C M, et al. Chromosomal mapping of members of the cdc2 family of protein kinases, cdk3, cdk6, PISSLRE, and PITALRE, and a cdk inhibitor, p27Kip1, to regions involved in human cancer. Cancer Res. (1995) 55(6):1199-205.

Burton D R, Barbas C F 3rd. Human antibodies from combinatorial libraries. Adv Immunol. (1994) 57:191-280. Review.

Caldas C, Hahn S A, da Costa L T, Redston M S, Schutte M, Seymour A B, Weinstein C L, Hruban R H, Yeo C J, Kern S E. Frequent somatic mutations and homozygous deletions of the p16 (MTS1) gene in pancreatic adenocarcinoma. Nat Genet. 1994 Sep;8(1):27-32.

Carbonelli D L, Corley E, Seigelchifer M, Zorzopulos J. A plasmid vector for isolation of strong promoters in Escherichia coli. FEMS Microbiol Lett. (1999) 177(1):75-82.

Cardone, M. H., et al. (1998) Regulation of cell death protease caspase-9 by phosphosphorylation. Science 282, 1318-1321.

Chandler S D, Mayeda A, Yeakley J M, Kramer A R, Fu X D. Free in PMC RNA splicing specificity determined by the coordinated action of RNA recognition motifs in SR proteins. Proc Natl Acad Sci U S A. (1997) 94(8):3596-601.

Chen, J., Jackson, P. K., Kirschner, M. W., & Dutta, A. (1995) Separate domains of p2l involved in the inhibition of Cdk kinase and PCNA. Nature 374, 386-388.

Cheng J Q, Jhanwar S C, Klein W M, Bell D W, Lee W C, Altomare D A, Nobori T, Olopade O I, Buckler A J, Testa J R. p 16 alterations and deletion mapping of 9p2 1-p22 in malignant mesothelioma. Cancer Res. (1994) 54(21):5547-51.

Cleary M L, Sklar J. Formation of a hybrid bcl-2/immunoglobulin transcript as a result of t(14;18) chromosomal translocation. Hamatol Bluttransfus. (1987) 31:314-9.

Cleary M L, Smith S D, Sklar J. Cloning and structural analysis of cDNAs for bcl-2 and a hybrid bcl-2/immunoglobulin transcript resulting from the t(14;18) translocation. Cell. (1986) 47(1):19-28.

Cocea L. Duplication of a region in the multiple cloning site of a plasmid vector to enhance cloning-mediated addition of restriction sites to a DNA fragment. Biotechniques. (1997) 23(5):814-6.

Coffin J M. Molecular mechanisms of nucleic acid integration. J Med Virol. (1990) 31(1):43-9.

Cotten M, Wagner E, Zatloukal K, Phillips S, Curiel D T, Birnstiel M L. High-efficiency receptor-mediated delivery of small and large (48 kilobase gene constructs using the endosome-disruption activity of defective or chemically inactivated adenovirus particles. Proc Natl Acad Sci U S A. (1992) 89(13):6094-8.

Couch et al., 1963

Coupar B E, Andrew M E, Boyle D B. A general method for the construction of recombinant vaccinia viruses expressing multiple foreign genes. Gene. (1988) 68(1): 1-10.

Coussens L, Yang-Feng T L, Liao Y C, Chen E, Gray A, McGrath J, Seeburg P H, Libermann T A, Schlessinger J, Francke U, et al. Tyrosine kinase receptor with extensive homology to EGF receptor shares chromosomal location with neu oncogene. Science. (1985) 230(4730):1132-9.

Curiel D T, Wagner E, Cotten M, Birnstiel M L, Agarwal S, Li C M, Loechel S, Hu PC. High-efficiency gene transfer mediated by adenovirus coupled to DNA-polylysine complexes.Hum Gene Ther. (1992) 3(2):147-54.

Datta, S. R., Brunet, A., & Greenberg, M. E. (1999) Cellular survival: a play in three Akts. Genes Dev. 13, 2905-2927.

Diehl, J. A., Cheng, M., Roussel, M. F., & Sherr, C. J. (1998) Glycogen synthase kinase-3b regulates cyclin D1 proteolysis and subcellular localization. Genes Dev. 12, 3499.

Downward, J. (1998) Mechanisms and consequences of activation of protein kinase B/Akt. Curr. Opin. Cell Biol. 10, 262-267.

El-Deiry, W. S., et al. (1993) WAF1, a potential mediator of p53 tumor suppression. Cell 75, 817-825.

Erhardt, J. A. and Pittman, R. N. (1998) Ectopic p21(WAF1) expression induces differentiation-specific cell cycle changes in PC12 cells characteristic of nerve growth factor treatment. J. Biol. Chem. 273: 23517-23523.

Fechheimer M, Boylan J F, Parker S, Sisken J E, Patel G L, Zimmer S G. Transfection of mammalian cells with plasmid DNA by scrape loading and sonication loading. Proc Natl Acad Sci U S A. (1987) 84(23):8463-7.

Ferkol T, Kaetzel C S, Davis P B. Gene transfer into respiratory epithelial cells by targeting the polymeric immunoglobulin receptor. J. Clin Invest. (1993) 92(5):2394-400.

Fraley R T, Fomari C S, Kaplan S. Entrapment of a bacterial plasmid in phospholipid vesicles: potential for gene transfer. Proc Natl Acad Sci U S A. (1979) 76(7):3348-52.

Flores-Rozas H, Kelman Z, Dean F B, Pan Z Q, Harper J W, Elledge S J, O'Donnell M, Hurwitz J. Cdk-interacting protein 1 directly binds with proliferating cell nuclear antigen and inhibits DNA replication catalyzed by the DNA polymerase delta holoenzyme. Proc Natl Acad Sci U S A. (1994) 91(18):8655-9.

Flotte T R, Barraza-Ortiz X, Solow R, Afione S A, Carter B J, Guggino W B. An improved system for packaging recombinant adeno-associated virus vectors capable of in vivo transduction. Gene Ther. (1995) 2(1):29-37.

Gao F H , Abee T, Konings W N. Mechanism of action of the peptide antibiotic nisin in liposomes and cytochrome c oxidase-containing proteoliposomes. Appl Environ Microbiol. (1991) 57(8):2164-70.

Gartel, A. L. and Tyner, A. L. (1999) Transcriptional regulation of the p21((WAF1/CIP1)) gene. Exp. Cell. Res. 246, 280-289.

Gatti, A. and Traugh, T. A. (1999) A two-dimensional peptide gel electrophoresis system for phosphopeptide mapping and amino acid sequencing. Analyt. Biochem. 266, 198-204.

Gervais, J. L., et al. (1999) Cleavage of CDK inhibitor p21(Cip1/Waf1) by caspases is an early event during DNA damage-induced apoptosis. J. Biol. Chem. 273:19207-19212.

Gomez-Foix A M, Coats W S, Baque S, Alam T, Gerard R D, Newgard C B. Adenovirus-mediated transfer of the muscle glycogen phosphorylase gene into hepatocytes confers altered regulation of glycogen metabolism. J. Biol. Chem. (1992) 267(35):25129-34.

Goubin, F. & Ducommun, B. (1995) Identification of binding domains on the p21Cipl cyclin-dependent kinase inhibitor. Oncogene 10, 2281-2287.

Ghosh-Choudhury G, Graham F L. Stable transfer of a mouse dihydrofolate reductase gene into a deficient cell line using human adenovirus vector. Biochem Biophys Res Commun. (1987) 147(3):964-73.

Graham F L, Prevec L. Methods for construction of adenovirus vectors. Mol Biotechnol. (1995) 3(3):207-20.

Graham F L, van der Eb A J. A new technique for the assay of infectivity of human adenovirus 5 DNA. Virology. (1973) 52(2):456-67.

Gribskov M, Burgess R R, Devereux J. PEPPLOT, a protein secondary structure analysis program for the UWGCG sequence analysis software package. Nucleic Acids Res. (1986) 14(1):327-34.

Grunhaus and Horwitz, 1992

Guan K L, Jenkins C W, Li Y, Nichols M A, Wu X, O'Keefe C L, Matera A G, Xiong Y. Growth suppression by p 18, a p 161NK4/MTS1- and p 141NK4B/MTS2-related CDK6 inhibitor, correlates with wild-type pRb function. Genes Dev. (1994) 8(24):2939-52.

Hannon G J, Beach D. p15INK4B is a potential effector of TGF-beta-induced cell cycle arrest. Nature. (1994) 371(6494):257-61.

Harland R, Weintraub H. Translation of mRNA injected into Xenopus oocytes is specifically inhibited by antisense RNA. J Cell Biol. (1985) 101(3):1094-9.

Harper, J. W., Adami, G. R., Wei, N., Keyomarsi, K., & Elledge, S. J. (1993) The p21 Cdk-interacting protein Cip is a potent inhibitor of GI cyclin-dependent kinase. Cell 75, 805-816.

Hermonat P L, Muzyczka N. Use of adeno-associated virus as a mammalian DNA cloning vector: transduction of neomycin resistance into mammalian tissue culture cells. Proc Natl Acad Sci U S A. (1984) 81(20):6466-70.

Hersdorffer et al., 1990

Hilton D J, Watowich S S, Katz L, Lodish H F. Saturation mutagenesis of the WSXWS motif of the erythropoietin receptor. J. Biol. Chem. (1996) 271(9):4699-708.

Hollstein M, Sidransky D, Vogelstein B, Harris CC. p53 mutations in human cancers. Science. (1991) 253(5015):49-53

Horwich A L, Furtak K, Pugh J, Summers J. Synthesis of hepadnavirus particles that contain replication-defective duck hepatitis B virus genomes in cultured HuH7 cells. J Virol. (1990) 64(2):642-50.

Hudziak R M, Schlessinger J, Ullrich A. Increased expression of the putative growth factor receptor p185HER2 causes transformation and tumorigenesis of NIH 3T3 cells. Proc Natl Acad Sci U S A. (1987) 84(20):7159-63.

Hung M C, Schechter A L, Chevray P Y, Stern D F, Weinberg R A. Molecular cloning of the neu gene: absence of gross structural alteration in oncogenic alleles. Proc Natl Acad Sci U S A. (1986) 83(2):261-4.

Hung M C, Yan D H, Zhao X Y. Amplification of the proto-neu oncogene facilitates oncogenic activation by a single point mutation. Proc Natl Acad Sci U S A. (1989) 86(8):2545-8.

Hussussian C J, Struewing J P, Goldstein A M, Higgins P A, Ally D S, Sheahan M D, Clark W H Jr, Tucker M A, Dracopoli N C. Gennline p16 mutations in familial melanoma. Nat Genet. (1994) 8(1):15-21.

Jarpe et al. (1998) Anti-apoptotic versus pro-apoptotic signal transduction: checkpoints and stop signs along the road to death. Oncogene 17, 1475-1482.

Jen J, Harper J W, Bigner S H, Bigner D D, Papadopoulos N, Markowitz S, Willson J K, Kinzler K W, Vogelstein B. Deletion of p16 and p15 genes in brain tumors. Cancer Res. (1994) 54(24):6353-8.

Jiang, Y. and Porter, A. G. (1998) Prevention of tumor necrosis factor (TNF)-mediated induction of p21WAF1/CIP1 sensitizes MCF-7 carcinoma cells to TNF-induced apoptosis. Biochem. Biophys. Res. Commun. 245, 691-697.

Jones N, Shenk T. Related Articles An adenovirus type 5 early gene function regulates expression of other early viral genes. Proc Natl Acad Sci U S A. (1979) 76(8):3665-9.

Kamb A, Shattuck-Eidens D, Eeles R, Liu Q, Gruis N A, Ding W, Hussey C, Tran T, Miki Y, Weaver-Feldhaus J, et al. Analysis of the p16 gene (CDKN2) as a candidate for the chromosome 9p melanoma susceptibility locus. Nat Genet. (1994) 8(1):23-6.

Kaneda Y, Iwai K, Uchida T. Introduction and expression of the human insulin gene in adult rat liver. J. Biol. Chem. (1989) 264(21):12126-9.

Kelleher Z T, Vos J M. Long-term episomal gene delivery in human lymphoid cells using human and avian adenoviral-assisted transfection. Biotechniques. (1994) 17(6):1110-7.

Kerr J F, Wyllie A H, Currie A R. Apoptosis: a basic biological phenomenon with wide-ranging implications in tissue kinetics. Br J Cancer. (1972) 26(4):239-57.

Klein et al., 1987

Kops, G. J. P. L., et al. (1999) Direct control of the forkhead transcription factor AFX by protein kinase B. Nature 398, 630-634.

Kotin R M, Siniscalco M, Samulski R J, Zhu X D, Hunter L, Laughlin C A, McLaughlin S, Muzyczka N, Rocchi M, Berns K I. Site-specific integration by adeno-associated virus. Proc Natl Acad Sci U S A. (1990) 87(6):2211-5.

Kraus J, Woltje M, Schonwetter N, Hollt V. Alternative promoter usage and tissue specific expression of the mouse somatostatin receptor 2 gene. FEBS Lett. (1998) 428(3): 165-70.

Lareyre J J, Thomas T Z, Zheng W L, Kasper S, Ong D E, Orgebin-Crist M C, Matusik R J. A 5-kilobase pair promoter fragment of the murine epididymal retinoic acid-binding protein gene drives the tissue-specific, cell-specific, and androgen-regulated expression of a foreign gene in the epididymis of transgenic mice. J. Biol. Chem. (1999) 274(12):8282-90.

Lebkowski J S, McNally M M, Okarma T B, Lerch L B. Adeno-associated virus: a vector system for efficient introduction and integration of DNA into a variety of mammalian cell types. Mol Cell Biol. (1988) 8(10):3988-96.

Le Gal La Salle G, Robert J J, Berrard S, Ridoux V, Stratford-Perricaudet LD, Perricaudet M, Mallet J. An adenovirus vector for gene transfer into neurons and glia in the brain. Science. (1993) 12;259(5097):988-90.

Levkau, B. et al. (1998) Cleavage of p21^{Cip1/Waf1} and p27^{Kip1} mediates apoptosis in endothelial cells through activation of Cdk2: role of a caspase cascade. Mol. Cell. 1, 553-563.

Levrero M, Barban V, Manteca S, Ballay A, Balsamo C, Avantaggiati M L, Natoli G, Skellekens H, Tiollais P, Perricaudet M. Defective and nondefective adenovirus vectors for expressing foreign genes in vitro and in vivo.Gene. (1991) 101(2):195-202.

Li R, Waga S, Hannon G J, Beach D, Stillman B. Differential effects by the p21 CDK inhibitor on PCNA-dependent DNA replication and repair. Nature. (1994) 371(6497):534-7.

Macejak DG, Sarnow P. Internal initiation of translation mediated by the 5′ leader of a cellular mRNA. Nature. (1991) 353(6339):90-4.

Mann J R, Corkery J J, Fisher H J, Cameron A H, Mayerova A, Wolf U, Kennaugh A A, Woolley V. The X linked recessive form of XY gonadal dysgenesis with a high incidence of gonadal germ cell tumours: clinical and genetic studies. J Med Genet. (1983) 20(4):264-70.

Markowitz D, Goff S, Bank A. A safe packaging line for gene transfer: separating viral genes on two different plasmids. J Virol. (1988) 62(4):1120-4.

McCarty D M, Christensen M, Muzyczka N. Sequences required for coordinate induction of adeno-associated virus p19 and p40 promoters by Rep protein. J Virol. (1991) 65(6):2936-45.

McLaughlin S K, Collis P, Hermonat P L, Muzyczka N. Adeno-associated virus general transduction vectors: analysis of proviral structures. J Virol. (1988) 62(6):1963-73.

Medema, R. H., Kops, G. J. P. L., Bos, J. L., & Burgering, B. M. T. (2000) AFX-like forkhead transcription factors mediate cell-cycle regulation by Ras and PKB through p27Kip1. Nature 404, 782-787.

Meier, R., Alessi, D. R., Cron, P., Andjelkovic, M., & Hemmings, B. A. (1997) Mitogenic activation, phosphorylation and nuclear translocation of protein kinase B. J. Biol. Chem. 272, 30491-30497.

Mori et al., 1994

Muller W J, Sinn E, Pattengale P K, Wallace R, Leder P. Single-step induction of mammary adenocarcinoma in transgenic mice bearing the activated c-neu oncogene. Cell. (1988) 54(1):105-15.

Muthuswamy S K, Siegel P M, Dankort D L, Webster M A, Muller W J. Mammary tumors expressing the neu proto-oncogene possess elevated c-Src tyrosine kinase activity. Mol Cell Biol. (1994) 14(1):735-43.

Muzyczka N. Use of adeno-associated virus as a general transduction vector for mammalian cells. Curr Top Microbiol Immunol. (1992) 158:97-129.

Nabel, G., Yang, Z. -Y., and Nabel, E (1998) Methods for treating cancers and restenosis with p21.

Needleman S B, Wunsch C D. A general method applicable to the search for similarities in the amino acid sequence of two proteins. J. Mol. Biol. (1970) 48(3):443-53.

Nicolas J F, Rubenstein J L. Retroviral vectors. Biotechnology. (1988) 10:493-513.

Nicolau C, Sene C. Liposome-mediated DNA transfer in eukaryotic cells. Dependence of the transfer efficiency upon the type of liposomes used and the host cell cycle stage. Biochim Biophys Acta. (1982) 721(2):185-90.

Nicolau C, Legrand A, Grosse E. Liposomes as carriers for in vivo gene transfer and expression. Methods Enzymol. (1987) 149:157-76.

Nobori T, Miura K, Wu D J, Lois A, Takabayashi K, Carson D A. Deletions of the cyclin-dependent kinase-4 inhibitor gene in multiple human cancers. Nature. (1994) 368(6473):753-6.

Noda, A., Ning, Y., Venable, S. F., Pereira-Smith, O. M., and Smith, J. R. (1994) Cloning of senescent cell-derived inhibitors of DNA synthesis using an expression screen. Exp. Cell Res. 211, 90-98.

Nomoto S, Tatematsu Y, Takahashi T, Osada H. Protein, Nucleotide Cloning and characterization of the alternative promoter regions of the human LIMK2 gene responsible for alternative transcripts with tissue-specific expression. Gene. (1999) 236(2):259-71.

Okamoto A, Demetrick D J, Spillare E A, Hagiwara K, Hussain S P, Bennett W P, Forrester K, Gerwin B, Serrano M, Beach D H, et al. Mutations and altered expression of p161NK4 in human cancer. Proc Natl Acad Sci U S A. (1994) 91(23):11045-9.

Ohi S, Dixit M, Tillery M K, Plonk S G. Construction and replication of an adeno-associated virus expression vector that contains human beta-globin cDNA.Gene. (1990) 89(2):279-82.

Orlow I, Lianes P, Lacombe L, Dalbagni G, Reuter V E, Cordon-Cardo C. Chromosome 9 allelic losses and microsatellite alterations in human bladder tumors. Cancer Res. (1994) 54(11):2848-51.

Ozes, O. N., et al. (1999) NF-kB activation by tumor necrosis factor requires the Akt serine-threonine kinase. Nature 401, 82-85.

Park, J. A. et al. (1998) Caspase 3 specifically cleaves p21 ^{WAF1/CIP1} in the earlier stage of apoptosis in SK-HEP-1 human hepatoma cells. Eur. J. Biochem. 257, 242-248.

Paskind M P, Weinberg R A, Baltimore D. Dependence of Moloney murine leukemia virus production on cell growth. Virology. (1975) 67(1):242-8.

Pelletier J, Sonenberg N. Internal initiation of translation of eukaryotic mRNA directed by a sequence derived from poliovirus RNA. Nature. (1988) 334(6180):320-5.

Perales J C, Ferkol T, Molas M, Hanson R W. An evaluation of receptor-mediated gene transfer using synthetic DNA-ligand complexes. Eur J Biochem. (1994) 226(2):255-66.

Peso, L. D., Gonzalez-Garcia, M., Page, C., Herrera, R., and Nunez, G. (1997) Interleukin-3-induced phosphorylation of bad through the protein kinase Akt. Science 278, 687-689.

Pietenpol J A, Bohlander S K, Sato Y, Papadopoulos N, Liu B, Friedman C, Trask B J, Roberts J M, Kinzler K W, Rowley J D, et al. Assignment of the human p27Kip1 gene to 12p 13 and its analysis in leukemias. Cancer Res. (1995) 55(6):1206-10.

Polyak K, Kato J Y, Solomon M J, Sherr C J, Massague J, Roberts J M, Koff A. p27Kip1, a cyclin-Cdk inhibitor, links transforming growth factor-beta and contact inhibition to cell cycle arrest.Genes Dev. (1994) 8(1):9-22.

Ponce-Castaneda M V, Lee M H, Latres E, Polyak K, Lacombe L, Montgomery K, Mathew S, Krauter K, Sheinfeld J, Massague J, et al. p27Kip1: chromosomal mapping to 12p12-12p 13.1 and absence of mutations in human tumors. Cancer Res. (1995) 55(6):1211-4.

Porter, A. G. (1999) Protein translocation in apoptosis. Trends Cell Biol. 9, 394-401.

Potter H, Weir L, Leder P. Enhancer-dependent expression of human kappa immunoglobulin genes introduced into mouse pre-B lymphocytes by electroporation. Proc Natl Acad Sci U S A. (1984) 81(22):7161-5.

Ragot T, Vincent N, Chafey P, Vigne E, Gilgenkrantz H, Couton D, Cartaud J, Briand P, Kaplan J C, Perricaudet M, et al. Efficient adenovirus-mediated transfer of a human -minidystrophin gene to skeletal muscle of mdx mice. Nature. (1993) 361(6413):647-50.

Remington's Pharmaceutical Sciences, 18th Ed. Mack Printing Company, 1990.

Renan M J. Cancer genes: current status, future prospects, and applications in radiotherapy/oncology. Radiother Oncol. (1990) 19(3):197-218.

Rich D P, Couture L A, Cardoza L M, Guiggio V M, Armentanb D, Espino P C, Hehir K, Welsh M J, Smith A E, Gregory R J. Development and analysis of recombinant adenoviruses for gene therapy of cystic fibrosis. Hum Gene Ther. (1993) 4(4):461-76.

Rippe R A, Brenner D A, Leffert H L. DNA-mediated gene transfer into adult rat hepatocytes in primary culture. Mol Cell Biol. (1990) 10(2):689-95.

Romashkova, J. A., & Makarov, S. S. (1999) NF-kB is a target of Akt in anti-apoptotic PDGF signaling. Nature 401, 86-90.

Rosenfeld M A, Siegfried W, Yoshimura K, Yoneyama K, Fukayama M, Stier L E, Paakko P K, Gilardi P, Stratford-Perricaudet L D, Perricaudet M, et al. Adenovirus-mediated transfer of a recombinant alpha 1-antitrypsin gene to the lung epithelium in vivo. Science. (1991) 252(5004):431-4.

Rosenfeld M A, Yoshimura K, Trapnell B C, Yoneyama K, Rosenthal E R, Dalemans W, Fukayama M, Bargon J, Stier L E, Stratford-Perricaudet L, et al. In vivo transfer of the human cystic fibrosis transmembrane conductance regulator gene to the airway epithelium. Cell. (1992) 68(1):143-55.

Rousseau, D. et al. (1999) Growth inhibition by CDK-cyclin and PCNA binding domains of p21 occurs by distinct mechanisms and is regulated by ubiquitin-proteasome pathway. Oncogene 18, 3290-3302.

Roux P, Jeanteur P, Piechaczyk M. A versatile and potentially general approach to the targeting of specific cell types by retroviruses: application to the infection of human cells by means of major histocompatibility complex class I and class II antigens by mouse ecotropic murine leukemia virus-derived viruses. Proc Natl Acad Sci U S A. (1989) 86(23):9079-83.

Samulski RJ , Chang L S, Shenk T. Helper-free stocks of recombinant adeno-associated viruses: normal integration does not require viral gene expression. J Virol. 1989 Sep;63(9):3822-8.

Sanba et al., 1985

Schechter A L, Stem D F, Vaidyanathan L, Decker S J, Drebin J A, Greene M I, Weinberg R A. The neu oncogene: an erb-B-related gene encoding a 185,000-Mr tumour antigen. Nature. (1984) 312(5994):513-6.

Schneider P M, Hung M C, Chiocca S M, Manning J, Zhao X Y, Fang K, Roth J A. Differential expression of the c-erbB-2 gene in human small cell and non-small cell lung cancer. Cancer Res. (1989) 49(18):4968-71.

Schwartz et al., 1979

Schwarze S R, Ho A, Vocero-Akbani A, Dowdy S F. In vivo protein transduction: delivery of a biologically active protein into the mouse. Science. (1999) 285(5433):1569-72.

Scott, M. T., Morrice, N., and Ball, K. L. (2000) Reversible phosphorylation at the c-terminal regulatory domain of p21Waf1/Cip1 modulates proliferating cell nuclear antigen binding. J. Biol. Chem. 275, 11529-11537.

Serrano M, Hannon G J, Beach D. A new regulatory motif in cell-cycle control causing specific inhibition of cyclin D/CDK4. Nature. (1993) 366(6456):704-7.

Shao, R., et al. (1997) Inhibition of nuclear factor-kB activity is involoved in E1 A-mediated sensitization of radioation-induced apoptosis. J. Biol. Chem. 272, 32739-32742.

Schechter A L, Stem D F, Vaidyanathan L, Decker S J, Drebin J A, Greene M I, Weinberg R A. The neu oncogene: an erb-B-related gene encoding a 185,000-Mr tumour antigen. Nature (1984) 312(5994):513-6.

Senba et al., 1985

Shelling A N, Smith M G. Targeted integration of transfected and infected adeno-associated virus vectors containing the neomycin resistance gene. Gene Ther. (1994) 1(3): 165-9.

Sherr, C. J. & Roberts, J. M. (1995) Inbhibitors of mammalian GI cyclin-dependent kinases. Genes Dev. 9, 1149-1163.

Sistonen L, Holtta E, Lehvaslaiho H, Lehtola L, Alitalo K. Activation of the neu tyrosine kinase induces the fos/jun transcription factor complex, the glucose transporter and ornithine decarboxylase. J Cell Biol. (1989) 109(5):1911-9.

Slamon, D. J., et al. (1987) Human breast cancer: correlation of relapse and survival with amplification of the HER-2/neu oncogene. Science 235, 177-182.

Slamon, D. J., et al. (1989) Studies of the HER-2/neu proto-oncogene in human breast and ovarian cancer. Science 244, 707-712.

Smith T F, Waterman M S, Fitch W M. Comparative biosequence metrics. J Mol Evol. (1981) 18(1):38-46.

Stratford-Perricaudet L D, Levrero M, Chasse J F, Perricaudet M, Briand P. Evaluation of the transfer and expression in mice of an enzyme-encoding gene using a human adenovirus vector. Hum Gene Ther. (1990) 1(3):241-56.

Stratford-Perricaudet L D, Briand P, Perricaudet M. Feasibility of adenovirus-mediated gene transfer in vivo. Bone Marrow Transplant. (1992) 9 Suppl 1:151-2.

Taules, M., et al. (1999) Calmodulin binds to p21Cip1 and is involved in the regulation of its nuclear localization. J. Biol. Chem. 274, 24445-24448.

Temin H M. Retroviruses and evolution. Cell Biophys. (1986) 9(1-2):9-16.

Top F H Jr, Buescher E L, Bancroft W H, Russell P K. Immunization with live types 7 and 4 adenovirus vaccines. II. Antibody response and protective effect against acute respiratory disease due to adenovirus type 7. J Infect Dis. (1971) 124(2):155-60.

Toyoshima H, Hunter T. p27, a novel inhibitor of G1 cyclin-Cdk protein kinase activity, is related to p21. Cell. (1994) 78(1):67-74. Tratschin J D, West M H, Sandbank T, Carter B J. A human parvovirus, adeno-associated virus, as a eucaryotic vector: transient expression and encapsidation of the procaryotic gene for chloramphenicol acetyltransferase. Mol Cell Biol. (1984) 4(10):2072-81.

Travali S, Koniecki J, Petralia S, Baserga R. Oncogenes in growth and development. FASEB J. (1990) 4(14):3209-14.

Tsai C M, Chang K T, Perng R P, Mitsudomi T, Chen M H, Kadoyama C, Gazdar A F. Correlation of intrinsic chemoresistance of non-small-cell lung cancer cell lines with HER-2/neu gene expression but not with ras gene mutations. J Natl Cancer Inst. (1993) 85(11):897-901.

Tsai C M, Yu D, Chang K T, Wu LH, Perng R P, Ibrahim N K, Hung M C. Enhanced chemoresistance by elevation of p185neu levels in HER-2/neu-transfected human lung cancer cells. J Natl Cancer Inst. (1995) 87(9):682-4.

Tsumaki N, Kimura T, Tanaka K, Kimura JH, Ochi T, Yamada Y. Modular arrangement of cartilage- and neural tissue-specific cis-elements in the mouse alpha2(XI) collagen promoter. J. Biol. Chem. (1998) 273(36):22861-4.

Tsujimoto Y, Croce CM. Analysis of the structure, transcripts, and protein products of bcl-2, the gene involved in human follicular lymphoma. Proc Natl Acad Sci U S A. (1986) 83(14):5214-8.

Tsujimoto Y, Cossman J, Jaffe E, Croce CM. Involvement of the bcl-2 gene in human follicular lymphoma. Science. (1985) 228(4706):1440-3.

Tur-Kaspa R, Teicher L, Levine B J, Skoultchi A I, Shafritz D A. Use of electroporation to introduce biologically active foreign genes into primary rat hepatocytes. Mol Cell Biol. (1986) 6(2):716-8.

Van der Geer, P. and Hunter, T. (1994) Phosphopeptides mapping and phosphoamino acid analysis by electrophoresis and chromatography on thin-layer cellulose plates. Electrophoresis 15, 544-554.

Varmus H E, Quintrell N, Ortiz S. Retroviruses as mutagens: insertion and excision of a nontransforming provirus alter expression of a resident transforming provirus. Cell. (1981) 25(1):23-36.

Wagner E, Zenke M, Cotten M, Beug H, Birnstiel M L. Transferrin-polycation conjugates as carriers for DNA uptake into cells. Proc Natl Acad Sci U S A. (1990) 87(9):3410-4.

Warren M S, Marolewski A E, Benkovic S J. A rapid screen of active site mutants in glycinamide ribonucleotide transformylase. Biochemistry. (1996) 35(27):8855-62.

Weiner D B, Nordberg J, Robinson R, Nowell P C, Gazdar A, Greene M I, Williams W V, Cohen J A, Kern J A. Expression of the neu gene-encoded protein (P185neu) in human non-small cell carcinomas of the lung. Cancer Res. (1990) 50(2):421-5.

Wells J A. Binding in the growth hormone receptor complex. Proc Natl Acad Sci U S A. (1996) 93(1):1-6.

Wong P, Hampton B, Szylobryt E, Gallagher A M, Jaye M, Burgess W H. Analysis of putative heparin-binding domains of fibroblast growth factor-1. Using site-directed mutagenesis and peptide analogues. J. Biol. Chem. (1995) 270(43):25805-11.

Wu G Y, Wu C H. Receptor-mediated in vitro gene transformation by a soluble DNA carrier system. J. Biol. Chem. (1987) 262(10):4429-32.

Wu G Y, Wu C H. Receptor-mediated gene delivery and expression in vivo. J. Biol. Chem. (1988) 263(29):14621-4.

Xiong Y, Zhang H, Beach D. D type cyclins associate with multiple protein kinases and the DNA replication and repair factor PCNA. Cell. (1992) 71(3):505-14.

Xiong Y, Zhang H, Beach D. Subunit rearrangement of the cyclin-dependent kinases is associated with cellular transformation.Genes Dev. (1993) 7(8):1572-83.

Xiong Y, Hannon G J, Zhang H, Casso D, Kobayashi R, Beach D. p21 is a universal inhibitor of cyclin kinases. Nature. (1993) 366(6456):701-4.

Yamamoto T, Ikawa S, Akiyama T, Semba K, Nomura N, Miyajima N, Saito T, Toyoshima K. Similarity of protein encoded by the human c-erb-B-2 gene to epidermal growth factor receptor. Nature (1986) 319(6050):230-4.

Yelton D E, Rosok M J, Cruz G, Cosand W L, Bajorath J, Hellstrom I, Hellstrom K E, Huse W D, Glaser S M. Affinity maturation of the BR96 anti-carcinoma antibody by codon-based mutagenesis. J. Immunol. (1995) 155(4):1994-2004.

Yu, D. & Hung, M. -C. (2000) Therapeutic resistance of erbB2-overexpressing cancers and strategies to overcome this resistance, in DNA Alterations in Cancer, M. Ehrlich, Eds. (Natick, Mass., 2000), Ch. 21.

Yu, D., et al. (1998) Overexpressing of ErbB-2 blocks taxol-induced apoptosis by upregulation of p21Cipl, which inhibits p34Cdc2 kinase. Mol. Cell 2, 581-591.

Yu D, Wang S S, Dulski K M, Tsai C M, Nicolson G L, Hung M C. c-erbB-2/neu overexpression enhances metastatic potential of human lung cancer cells by induction of metastasis-associated properties. Cancer Res. (1994) 54(12):3260-6.

Yu D, Wolf J K, Scanlon M, Price J E, Hung M C Enhanced c-erbB-2/neu expression in human ovarian cancer cells correlates with more severe malignancy that can be suppressed by E1A. Cancer Res. (1993) 53(4):891-8.

Yu D H, Hung M C. Expression of activated rat neu oncogene is sufficient to induce experimental metastasis in 3T3 cells. Oncogene. (1991) 6(11):1991-6.

Zelenin A V, Alimov A A, Barmintzev V A, Beniumov A O, Zelenina I A, Krasnov A M, Kolesnikov V A. The delivery of foreign genes into fertilized fish eggs using high-velocity microprojectiles. FEBS Lett. (1991) 287(1-2):118-20.

Zeng C, Aleshin A E, Hardie J B, Harrison R W, Fromm H J. ATP-binding site of human brain hexokinase as studied by molecular modeling and site-directed mutagenesis. Biochemistry. (1996) 35(40):13157-64.

Zhang et al. (1999) Caspase-mediated cleavage of p21^{WAF1/CIP1} converts cancer cells from growth arrest to undergoing apoptosis. Oncogene 18, 1131-1138.

Zhao-Emonet J C, Boyer O, Cohen J L, Klatzmann D. Deletional and mutational analyses of the human CD4 gene promoter: characterization of a minimal tissue-specific promoter. Biochim Biophys Acta. (1998) 1442(2-3):109-19.

Zhou, B. P., et al. (2000) HER-2/neu blocks tumor necrosis factor-induced apoptosis via the Akt/NF-kB pathways. J. Biol. Chem. 275, 8027-8031.

What is claimed is:

1. A p21^{Cip1/WAF1} polypeptide comprising an amino acid substitution at Thr¹⁴⁵.

2. The polypeptide of claim 1, wherein the substitution prevents phosphorylation of the p21^{Cip1/WAF1} polypeptide under conditions which would result in phosphorylation of an unsubstituted p21^{Cip1/WAF1} polypeptide.

3. The polypeptide of claim 1, wherein the substitution is a Thr¹⁴⁵ to Ala¹⁴⁵ substitution.

4. The polypeptide of claim 1, wherein the substitution is a Thr¹⁴⁵ to Asp145 substitution.

5. The polypeptide of claim 1, wherein Thr¹⁴⁵ is substituted with an amino acid other than any of aspartic acid, glutamic acid, or serine.

6. The polypeptide of matter of claim 1, wherein the substitution results in nuclear accumulation of the p21^{Cip1/WAF1} polypeptide following activation of Akt under conditions in which an unsubstituted p21^{Cip1/WAF1} polypeptide would translocate from the nucleus to the cytoplasm of a cell.

7. The polypeptide of claim 1, further defined as a composition in a pharmacologically acceptable excipient in which the p21^{Cip1/WAF1} polypeptide is dispersed.

8. The polypeptide claim 1, further defined as comprised in a pharmacologically acceptable excipient.

9. The polypeptide of matter of claim 1, further defined as being comprised in a suitable container in a kit.

10. A p21^{Cip1/WAF1} polypeptide comprising a modification which prohibits phosphorylation of the polypeptide under conditions which would result in phosphorylation of an unsubstituted p21^{Cip1/WAF1} polypeptide, wherein the modification results in accumulation of the polypeptide in a nucleus of a cell under conditions in which the unsubstituted p21^{Cip1/WAF1} polypeptide would translocate from the nucleus to the cytoplasm of the cell.

11. The polypeptide of claim 10, wherein the modification is an amino acid substitution at Thr¹⁴⁵.

12. A method comprising administering to a cell a p21^{Cip1/WAF1} polypeptide having an amino acid substitution at Thr¹⁴⁵.

13. The method of claim 12, wherein the substitution is a Thr¹⁴⁵ to Ala¹⁴⁵ substitution.

14. The method of claim 12, wherein the substitution is a Thr¹⁴⁵ to Asp¹⁴⁵ substitution.

15. The method of claim 12, wherein the Thr¹⁴⁵ is substituted with an amino acid other than aspartic acid, glutamic acid, and serine.

16. The method of claim 12, wherein the polypeptide further comprises a protein transduction domain.

17. The method of claim 12, further defined as a method of preventing cytoplasmic translocation of p21^{Cip1/WAF1} polypeptide from a nucleus of a cell following activation of Akt.

18. The method of claim 12, wherein the cell is comprised in an animal.

19. The method of claim 18, wherein the animal is a human.

20. The method of claim 19, wherein the human has a proliferative cell disorder.

21. The method of claim 20, wherein the proliferative cell disorder is cancer.

22. The method of claim 21, wherein the cancer is breast cancer.

23. The method of claim 21, wherein the cancer is associated with HER-2/neu-mediated cell proliferation.

24. The method of claim 20, wherein the proliferative cell disorder is restenosis.

25. The method of claim 12, wherein the polypeptide is comprised in pharmacologically acceptable excipient.

26. The method of claim 25, wherein the polypeptide is complexed with a lipid.

27. The method of claim 12, wherein administering to the cell a p21^{Cip1/WAF1} polypeptide having an amino acid substitution at Thr¹⁴⁵ comprises administering to the individual a nucleic acid encoding a p21^{Cip1/WAF1} polypeptide having an amino acid substitution at Thr¹⁴⁵.

28. The method of claim 27, wherein the nucleic acid is comprised in a plasmid, a retroviral vector, an adenoviral vector, an adeno-associated viral vector, or a liposome.

29. The method of claim 27, wherein the nucleic acid is dispered in a pharmacologically acceptable excipient.

30. The method of claim 12, further defined as a method of preventing growth of a cell in an individual

31. The method of claim 12, further defined as a method of increasing cell survival in an individual.

32. The method of claim 31, wherein the polypeptide is further defined as having a Thr¹⁴⁵ to Asp¹⁴⁵ substitution or a Thr¹⁴⁵ to Glu¹⁴⁵ substitution.

33. The method of claim 31, further defined as a method of treating an individual for a degenerative disease

34. The method of claim 33, wherein the polypeptide is further defined as having a Thr¹⁴⁵ to Asp¹⁴⁵ substitution.

35. The method of claim 33, wherein the degenerative disease is selected from the group consisting of multiple sclerosis, muscular dystrophy, Alzheimer's disease, focal lobar atrophies, including semantic dementia and dementia of frontal type, subcortical dementia, including progressive supranuclear palsy, Huntington's disease and Parkinson's disease, lumbar degenerative disk disease, amyotrophic lateral sclerosis, degenerative joint disease, arthritis, Creutzfeldt-Jakob disease, degenerative valve disease, retinal degenerative disease, Sorsby's fundus dystrophy, and macular degeneration.

36. The method of claim 12, further defined as a method of inhibiting angiogenesis.

37. The method of claim 36, wherein the substitution is a Thr¹⁴⁵ to Ala¹⁴⁵ substitution.

38. A method of obtaining a nuclear-retained polypeptide of p21^{Cip1/WAF1} which remains in the nucleus following activation of Akt.

39. The method of claim 38, further defined as comprising:
obtaining a polynucleotide which encodes a p21^{Cip1/WAF1} polypeptide; and
altering the polynucleotide to effect a modification of the polypeptide; wherein when the modified polypeptide remains in the nucleus following activation of Akt, the polypeptide is a nucleus-retained polypeptide of p21^{Cip1/WAF1}.

40. The method of claim 39, further defined as comprising modifying the p21^{Cip1/WAF1} polypeptide at amino acid position 145, wherein the modification results in an inability of amino acid to be phosphorylated.

41. The method of claim 39, wherein the modification is an amino acid substitution at Thr¹⁴⁵.

42. The method of claim 39, further comprising placing the polypeptide in a pharmacologically acceptable excipient.

43. The method of claim 42, further comprising using the polypeptide in the pharmacologically acceptable excipient to treat an animal.

44. The method of claim 43, wherein the animal has a proliferative cell disorder.

45. The method of claim 44, wherein the proliferative cell disorder is cancer.

46. The method of claim 45, wherein the cancer is breast cancer.

47. The method of claim 43, wherein the animal is a human.

48. A method of identifying a p21^{Cip1/WAF1} polypeptide which accumulates in the nucleus of a cell following activation by Akt, comprising:
altering the polypeptide; and
assaying the polypeptide for nuclear accumulation in the cell under conditions wherein an unmodified p21^{Cip1/WAF1} polypeptide translocalizes from the nucleus to the cytoplasm of the cell.
